(19) United States
(12) Patent Application Publication
(10) Pub. No.: US 2014/0005115 A1
(43) Pub. Date: Jan. 2, 2014

(54) LIPID CONJUGATES IN THE TREATMENT OF BRONCHITIS

(71) Applicant: YISSUM RESEARCH DEVELOPMENT COMPAN, (US)

(72) Inventor: Saul YEDGAR, Jerusalem (IL)

(73) Assignee: YISSUM RESEARCH DEVELOPMENT COMPANY, Jerusalem (IL)

(21) Appl. No.: 13/788,325

(22) Filed: Mar. 7, 2013

Related U.S. Application Data
(63) Continuation-in-part of application No. 13/316,592, filed on Dec. 12, 2011.
Application No. 13/316,592 is a continuation-in-part of application No. 11/285,375, filed on Nov. 23, 2005, now Pat. No. 8,076,312.
Application No. 11/285,375 is a continuation-in-part of application No. PCT/IL2005/001225, filed on Nov. 17, 2005.
Application No. 13/316,592 is a continuation-in-part of application No. 12/463,792, filed on May 11, 2009, now abandoned.
Application No. 12/463,792 is a continuation-in-part of application No. 12/997,014, filed on Dec. 9, 2010.

(51) Int. Cl. A61K 47/48

(52) U.S. Cl. CPC A61K 47/48092; A61K 47/48084
USPC 514/17.2; 514/54; 514/56; 514/57; 514/60; 514/59

(57) ABSTRACT

Provided herein are methods of treating, suppressing, inhibiting, or preventing bronchitis in a subject comprising the step of administering to a subject a compound comprising a lipid or phospholipid moiety bond to a physiologically acceptable monomer, dimer, oligomer, or polymer, and/or a pharmaceutically acceptable salt or a pharmaceutical product thereof.

### CROSS REFERENCE TO RELATED APPLICATIONS

This application is a continuation-in-part of U.S. application Ser. No. 13/316,592, filed Dec. 12, 2011, which is a continuation-in-part of U.S. application Ser. No. 11/285,375, filed Nov. 23, 2005, which is a continuation-in-part of PCT International Application Number PCT/IL2005/001225, filed Nov. 17, 2005. U.S. application Ser. No. 13/316,592 is also a continuation-in-part of U.S. application Ser. No. 12/463,792, filed May 11, 2009, and of U.S. application Ser. No. 12/997,014, filed Dec. 9, 2010, which claims the benefit of U.S. Provisional Application No. 61/177,083, filed May 11, 2009, which are all hereby incorporated by reference in their entirety.

### FIELD OF THE INVENTION

Provided herein are method of treating, suppressing, inhibiting, or preventing bronchitis in a subject comprising the step of administering to a subject a compound comprising a lipid or phospholipid moiety bond to a physiologically acceptable monomer, dimer, oligomer, or polymer, and/or a pharmaceutically acceptable salt or a pharmaceutical product thereof.

### BACKGROUND OF THE INVENTION

Lipid-conjugates having a pharmacological activity of inhibiting the enzyme phospholipase A2 (PLA2, EC 3.1.1.4) are known in the prior art. Phospholipase A2 catalyzes the breakdown of phospholipids at the sn-2 position to produce a fatty acid and a lysophospholipid. The activity of this enzyme has been correlated with various cell functions, particularly with the production of lipid mediators such as eicosanoid production (prostaglandins, thromboxanes and leukotrienes), platelet activating factor and lysophospholipids.

Glycosaminoglycans (GAG) are macro-molecules that protect the cell membrane from attacks or stimuli by a multitude of extra-cellular agents such as: Free radicals (ROS), exogenous PLA2, interleukins and other inflammatory mediators, allergens, growth factors, and degrading enzymes or invasion-promoting enzymes (e.g., heparinase, collagenase, heparanase, hyaluronidase). GAG enrichment assists in protecting cells from damage.

Since their inception, lipid-conjugates have been subjected to intensive laboratory investigation in order to obtain a wider scope of protection of cells and organisms from injurious agents and pathogenic processes.

Bronchitis is an inflammation of the mucous membranes of the bronchi (the larger and medium-sized airways that carry airflow from the trachea into the more distal parts of the lung parenchyma). Bronchitis can be divided into two categories: acute and chronic.

Acute bronchitis is characterized by the development of a cough, with or without the production of sputum (mucus that is expectorated, or “coughed up”, from the respiratory tract). Acute bronchitis often occurs during the course of an acute viral illness such as the common cold or influenza. Viruses cause about 90% of acute bronchitis cases, whereas bacteria account for about 10%.

Chronic bronchitis, a type of COPD, is characterized by the presence of a productive cough that lasts for three months or more per year for at least two years. Chronic bronchitis usually develops due to recurrent injury to the airways caused by inhaled irritants. Cigarette smoking is the most common cause, followed by exposure to air pollutants such as sulfur dioxide or nitrogen dioxide, and occupational exposure to respiratory irritants. Individuals exposed to cigarette smoke, chemical lung irritants, or who are immunocompromised have an increased risk of developing bronchitis.

Among the goals of allergy treatment is to prevent the release of inflammatory mediators and thereby mitigate the symptoms associated with inflammation. There is a need for better treatment and management of bronchitis.

### SUMMARY OF THE INVENTION

In one aspect, methods are provided for treating bronchitis in a subject comprising the step of administering to said subject a compound represented by the structure of the general formula (A):

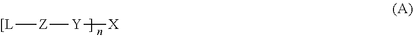

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer; and
n is a number from 1 to 1000.

In another aspect, methods are provided for preventing bronchitis in a subject, comprising the step of administering to said subject a compound represented by the structure of the general formula (A):

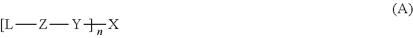

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer; and
n is a number from 1 to 1000.

In certain embodiments, X in general formula (A) is a polysaccharide. In some embodiments, the polysaccharide is carboxymethylcellulose, while in other embodiments, the polysaccharide is a glycosaminoglycan. In some embodiments, the glycosaminoglycan is hyaluronic acid, while in other embodiments, the glycosaminoglycan is heparin. In certain embodiments, L in general formula (A) is phosphatidylethanolamine, which in some embodiments is dipalmitoyl phosphatidylethanolamine.

According to one embodiment, provided is a method of treating or preventing bronchitis in a subject, comprising the step of administering to the subject a composition comprising a compound represented by the structure of the general formula (I):

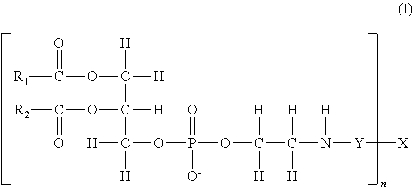

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is glycosaminoglycan, alginate, carboxymethylcellulose, or polygeline; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

The bronchitis according to some embodiments may be allergic bronchitis, acute bronchitis, or chronic bronchitis.

According to one embodiment, provided is a method for ameliorating broncho constriction in a subject, comprising the step of subjecting the subject to a composition comprising a compound represented by the structure of the general formula (I):

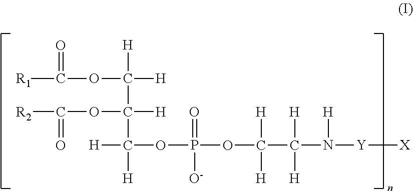

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is glycosaminoglycan, alginate, carboxymethylcellulose, or polygeline; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

Yet according to one embodiment, provided is a method for inhibiting contraction of a muscle tissue, comprising the step of subjecting the muscle tissue to a composition comprising a compound represented by the structure of the general formula (I):

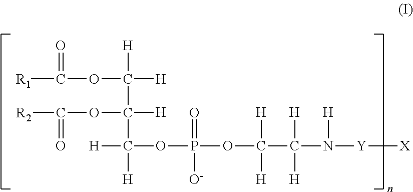

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is glycosaminoglycan, alginate, carboxymethylcellulose, or polygeline; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

In some embodiments, n may be a number from 2 to 1,000.

In some embodiments, the glycosaminoglycan may be selected from the group consisting of hyaluronic acid, heparin, heparan sulfate, chondroitin sulfate, keratan, keratan sulfate, dermatan sulfate or a derivative thereof.

In some embodiments, the phosphatidylethanolamine is a myristoyl or palmitoyl phosphatidylethanolamine.

In some embodiments, the phosphatidylethanolamine is a dipalmitoyl phosphatidylethanolamine, or dimyristoyl phosphatidylethanolamine.

In some embodiments, the composition is administered as aerosol.

In some embodiments, the composition is administered by inhalation.

In some embodiments, the composition is administered by intranasal administration.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description. It is also contemplated that whenever appropriate, any embodiment of the present invention can be combined with one or more other embodiments of the present invention, even though the embodiments are described under different aspects of the present invention.

### BRIEF DESCRIPTION OF FIGURES

FIG. 1 depicts the study design of the clinical trial described in Example 1.

FIG. 2. Plots of the mean (normalised) IL-5 levels at Day 21 for the Placebo, HyPE (Drug) and steroid (INS) groups, respectively.

FIG. 3. Plots of the mean (normalised) IL-13 levels at Day 21 for the Placebo, HyPE (Drug) and steroid (INS) groups, respectively.

FIG. 4. Plots of the mean (normalised) MCP-1 levels at Day 21 for the Placebo, HyPE (Drug) and steroid (INS) groups, respectively.

FIG. 5. Plots of the mean (normalised) TNF-α levels at Day 21 for the Placebo, HyPE (Drug) and steroid (INS) groups, respectively.

FIG. 6. Plots of the mean (normalised) IL-8 levels at Day 21 for the Placebo, HyPE (Drug) and steroid (INS) groups, respectively.

FIG. 7. Plots of the mean (normalised) Eotaxin levels at Day 21 for the Placebo, HyPE (Drug) and steroid (INS) groups, respectively.

FIG. 8. Plots of the mean (normalised) eosinophils at Day 21 for the Placebo, HyPE (Drug) and steroid (INS) groups, respectively.

FIG. 9. Bar graph comparing percentage of patients showing symptom improvement between the HyPE (Drug) and Placebo groups.

FIG. 10.1: Inhibition of endothelin-1 (ET)-induced contraction of rat tracheal rings by Lipid-conjugates. A: Contraction of rat trachea by Endothelin-1. B: Effect of HyPE on ET-induced contraction of rat trachea.

FIG. 10.2: Effect of HyPE and Hyaluronic acid (HA) on ET-1 induced contraction of rat to trachea.

FIG. 10.3: Effect of HyPE and Hyaluronic acid (HA) on Acetylcholine (AcCh)-induced contraction of isolated rat trachea rings.

FIG. 10.4: Effect of HyPE, administered subcutaneously, on early asthmatic reaction (EAR) induced by ovalbumin inhalation

FIG. 10.5: Effect of HyPE on sPLA₂ expression in lung of rats with OVA-induced asthma.

FIG. 10.6: Effect of HyPE on cysteinyl leukotriens (LTC₄, LTD₄ and LTE₄) level in the BAL of OVA-induced asthmatic rats.

FIG. 10.7: Effect of HyPE inhalation on early and late asthmatic reaction (EAR and LAR, respectively) in OVA-sensitized asthmatic rats.

FIG. 10.8: Effect of HyPE inhalation on cysteinyl leukotrienes (LTC4, LTD4 and LTE4) level in the BAL of OVA-sensitized asthmatic rats.

FIG. 10.9: Effect of HyPE inhalation on NO production by macrophages collected from the BAL of OVA-sensitized asthmatic rats.

FIG. 10.10: Effect of HyPE inhalation on structural change in airways (airway remodeling) of OVA sensitized asthmatic rats.

FIG. 10.11: Effect of HyPE on remodeling of asthmatic rat airway; histological morphometry.

FIG. 10.12: Effect of HyPE inhalation on TNFα production by macrophages collected from the BAL of OVA-sensitized asthmatic rats.

FIG. 10.13: Amelioration of OVA-induced broncho-constriction by HyPE inhalation before challenge.

FIG. 10.14: Amelioration of OVA-induced broncho-constriction by HyPE inhalation after challenge.

FIG. 10.15: Airway resistance of EAB mice following methacholine challenge. Mice with OVA-induced EAB (EAB-Mice), with/without treatment with sPLA₂ inhibitor (EAB and EAB/HyPE, respectively), were challenged with methacholine. Airway resistance was determined as described in Methods. Data are mean±SEM for 8 mice. *, P<0.01 for the highest dose.

FIG. 10.16: mRNA expression of arginase-I and acidic chitinase in lungs of EAB mice: mRNA of arginase-I and acidic chitinase in lungs was determined by RT-PCR. Each datum is mean±SEM for 10 mice in a group. *, #P<0.05.

FIG. 10.17A: Airway Resistance of EAB mice following OVA challenge: Mice were subjected to OVA challenge and airway response was determined as described in Methods Data are mean±SEM for 8 replications. *, #P<0.05.

FIG. 10.17B: Pulmonary enhancement (Penh) of EAB mice following OVA challenge: Mice were subjected to OVA challenge and airway response was determined by the change in Penh as described in Methods. The results are mean±SEM for 10 mice *, #P<0.01.

FIG. 10.18A: Representative histological micrographs of lungs of EAB mice: Mice lung tissues were stained with hematoxylin and eosin. A: Healthy mice B: Untreated EAB mice. C: EAB mice treated with HyPE.

FIG. 10.18B: Peri-bronchial infiltration of inflammatory cells in lungs of EAB Mice: The number of leukocytes in lung peri-bronchial space was determined by morphometry. A: Healthy mice B: Untreated EAB mice. C: EAB mice treated with HyPE. Data are mean±SEM for 10 mice.*, #P<0.01.

FIG. 10.19: mRNA expression of PLA₂s in EAB mice lung: mRNA of PLA₂s in mice lung homogenates was determined by RT-PCR. Each datum is mean±SEM for 10 mice in a group. Significant difference between naïve and EAB (P<0.01), and between EAB and EAB/HyPE (P<0.05) was found for sPLA₂gX and for cPLA₂gIVC. No significant difference was found for sPLA₂gV.

FIG. 10.20: Eicosanoid level in BAL of EAB mice: Eicosanoids in the mice BAL were determined by ELISA. Results are percent change relative to control (100%). The absolute control levels (100%) were 51.47 pg/ml for Cys-LTs, 101.83 ng/ml for TXB₂, 7.85 ng/ml for PGE₂ and 378.11 pg/ml for PGD₂. Data are mean±SEM for 10 mice. *, #, P<0.05; $, &, P<0.01.

FIG. 10.21: 5-LO protein level in EAB mice lung: 5-LO protein in mice lung homogenates was determined by Western blotting A. Representative blots. B. Blot quantification by densitometry, normalized to GAPDH. Data are mean±SEM for 3 independent experiments, normalized to GAPDH. Data are mean±SEM for 3 independent experiments. *P<0.05.

FIG. 11.1: CMPE protects BGM cells from membrane lysis induced by combined action of hydrogen peroxide (produced by glucose oxidase=GO), and exogenous phospholipase A₂ (PLA₂).

FIG. 11.2: CMPE protects BGM cells from glycosaminoglycan degradation by Hydrogen peroxide (produced by GO).

FIG. 11.3: HyPE protects LDL from copper-induced oxidation.

FIG. 12.1: Effect of different Lipid-conjugates on LPS-induced IL-8 production.

FIG. 12.2: Effect of HyPE on LPS-induced chemokine production.

FIG. 12.3: Effect of HyPE on LTA-induced IL-8 production.

FIG. 12.4: Effect of HyPE on LPS-induced ICAM-1 and E-selectin expression.

FIG. 12.5: Effect of HyPE on LPS-induced activation of NF-kB in LMVEC.

### DETAILED DESCRIPTION OF THE INVENTION

Disclosed herein are novel methods of use for lipid-conjugates which display a wide-range combination of cytoprotective pharmacological activities. These compounds can alleviate airway obstruction in asthma, protect mucosal tissue in gastrointestinal disease, suppress immune responses, alleviate cutaneous hypersensitivity reactions, inhibit cell proliferation associated with vascular injury and immunological responses, inhibit cell migration associated with vascular and central nervous system disease, attenuate oxidative damage to tissue proteins and cell membranes, interfere with viral spread, reduce tissue destroying enzyme activity, and reduce intracellular levels of chemokines and cytokines. Thus these compounds are useful in the treatment of a diversity of disease states, including asthma, bronchitis, rhinitis, allergic rhinitis, chronic obstructive pulmonary disease, obstructive respiratory disease, colitis, Crohn's disease, central nervous system insult, multiple sclerosis, contact dermatitis, psoriasis, cardiovascular disease, invasive medical procedures, invasive cellular proliferative disorders, anti-oxidant therapy, hemolytic syndromes, sepsis, acute respiratory distress syndrome, tissue transplant rejection syndromes, autoimmune disease, viral infection, and hypersensitivity conjunctivitis.

In certain embodiments, methods are provided of treating an obstructive respiratory disease in a subject comprising the step of administering to said subject a compound represented by the structure of the general formula (A):

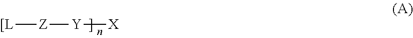

wherein
L is a lipid or a phospholipid;
Z is either nothing, ethanolamine, serine, inositol, choline, or glycerol;
Y is either nothing or a spacer group ranging in length from 2 to 30 atoms:
X is a physiologically acceptable monomer, dimer, oligomer, or polymer; and
n is a number from 1 to 1000.

In certain embodiments, methods are provided of preventing an obstructive respiratory disease in a subject, comprising the step of administering to said subject a compound represented by the structure of the general formula (A):

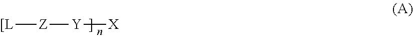

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer; and
n is a number from 1 to 1000.

In certain of the foregoing embodiments, the obstructive respiratory disease is rhinosinusitis. In other embodiments, the obstructive respiratory disease comprises a physical or anatomical obstruction, which in some embodiments, is a nasal polyp. In some embodiments, the obstructive respiratory disease is rhinitis. In yet other embodiments, the obstructive respiratory disease is sinusitis. In certain other embodiments, the obstructive respiratory disease is asthma. In certain other embodiments, the obstructive respiratory disease is bronchitis. In certain embodiments, the obstructive respiratory disease is allergic rhinitis. In certain other embodiments, the obstructive respiratory disease is chronic obstructive pulmonary disorder. In yet further embodiments, the obstructive respiratory disease is nasal polyposis.

In certain embodiments, methods are provided for treating bronchitis in a subject comprising the step of administering to said subject a compound represented by the structure of the general formula (A):

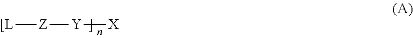

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer; and
n is a number from 1 to 1000.

In certain embodiments, methods are provided for preventing bronchitis in a subject, comprising the step of administering to said subject a compound represented by the structure of the general formula (A):

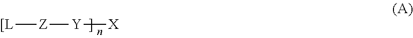

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer; and
n is a number from 1 to 1000.

In certain embodiments, X in general formula (A) is a polysaccharide. In some embodiments, the polysaccharide is carboxymethylcellulose, while in other embodiments, the polysaccharide is a glycosaminoglycan. In some embodiments, the glycosaminoglycan is hyaluronic acid, while in other embodiments, the glycosaminoglycan is heparin. In certain embodiments, L in general formula (A) is phosphatidylethanolamine, which in some embodiments is dipalmitoyl phosphatidylethanolamine.

In certain embodiments, the invention provides for the use of a compound represented by the structure of the general formula (A):

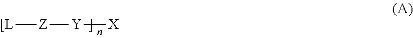

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer; and
n is a number from 1 to 1000
for the preparation of a composition to treat bronchitis.

In certain embodiments, the invention provides for the use of a compound represented by the structure of the general formula (A):

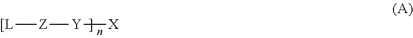

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer; and
n is a number from 1 to 1000
for the preparation of a composition to prevent bronchitis.

In certain embodiments, the invention provides for the use of a compound represented by the structure of the general formula (A):

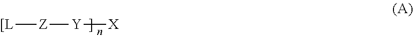

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer; and
n is a number from 1 to 1000
for treating bronchitis.

In certain embodiments, the invention provides for the use of a compound represented by the structure of the general formula (A):

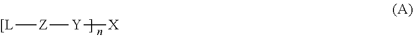

wherein
L is a lipid or a phospholipid;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer; and
n is a number from 1 to 1000 for preventing bronchitis.

In certain embodiments, compositions of the present invention may be used to treat, suppress, inhibit or prevent rhinosinusitis initially caused by a stimulus, such as an allergen, environmental stimulus, fungus, bacteria, or virus. In some embodiments, the bacterial infection is Staphylococcus Aureus. In some embodiments, the fungus or bacteria colonizes the sinus thereby causing an aggressive inflammatory reaction. In further embodiments, any of the stimuli described hereinabove leads to an inflammatory reaction of rhinosinusitis.

In certain embodiments, the invention provides methods of decreasing cytokine levels in a subject, comprising the step of administering to said subject a compound of the present invention. In some embodiments, the invention provides methods of returning elevated cytokine levels to basal levels in a subject, comprising the step of administering to said subject a compound of the present invention. In another embodiment, the invention provides methods of decreasing IL-13 levels in a subject, comprising the step of administering to said subject a compound of the present invention. In another embodiment, the invention provides methods of decreasing IL-5 levels in a subject, comprising the step of administering to said subject a compound of the present invention. In another embodiment, the invention provides methods of decreasing MCP-1 levels in a subject, comprising the step of administering to said subject a compound of the present invention. In another embodiment, the invention provides methods of decreasing TNF-α levels in a subject, comprising the step of administering to said subject a compound of the present invention. In another embodiment, the invention provides methods of decreasing IL-8 levels in a subject, comprising the step of administering to said subject a compound of the present invention. In another embodiment, the invention provides methods of decreasing eotaxin levels in a subject, comprising the step of administering to said subject a compound of the present invention. In another embodiment, the invention provides methods of decreasing interferon-γ levels in a subject, comprising the step of administering to said subject a compound of the present invention. In another embodiment, the invention provides methods of reversing increased IL-13 levels in a subject, comprising the step of administering to said subject a compound of the present invention. In another embodiment, the invention provides methods of reversing increased TL-5 levels in a subject, comprising the step of administering to said subject a compound of the present invention. In another embodiment, the invention provides methods of reversing increased MCP-1 levels in a subject, comprising the step of administering to said subject a compound of the present invention. In another embodiment, the invention provides methods of reversing increased TNF-α levels in a subject, comprising the step of administering to said subject a compound of the present invention. In another embodiment, the invention provides methods of reversing increased IL-8 levels in a subject, comprising the step of administering to said subject a compound of the present invention. In another embodiment, the invention provides methods of reversing increased eotaxin levels in a subject, comprising the step of administering to said subject a compound of the present invention. In another embodiment, the invention provides methods of reversing increased interferon-γ levels in a subject, comprising the step of administering to said subject a compound of the present invention.

In certain embodiments, X in general formula (A) is a polysaccharide. In some embodiments, the polysaccharide is carboxymethylcellulose, while in other embodiments, the polysaccharide is a glycosaminoglycan. In some embodiments, the glycosaminoglycan is hyaluronic acid, while in other embodiments, the glycosaminoglycan is heparin. In certain embodiments, L in general formula (A) is phosphatidylethanolamine, which in some embodiments is dipalmitoyl phosphatidylethanolamine.

In some embodiments, “treating” or “preventing” refers to delaying the onset of symptoms, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, preventing relapse to a disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics.

In some embodiments, the symptoms of bronchitis treated and/or prevented include one or more of the following symptoms: nasal congestion, rhinorrhea, frontal headache, post-nasal drip, sneezing, nasal itch, itching ears/palate and cough. In certain embodiments the symptoms of bronchitis treated and/or prevented include one or more of the following symptoms: nasal congestion, rhinorrhea, sneezing and nasal itch. In further embodiments, at least the symptom of coughing is treated and/or prevented; while, in other embodiments, at least the symptom of coughing is treated and/or prevented.

In some embodiments, treating and/or preventing bronchitis includes reducing the level of one or more of the following cytokines: IL-5, IL-13, MCP-1, TNF-α, IL-8 and eotaxin. In certain embodiments, treating or preventing bronchitis includes reducing eosinophil counts.

In some embodiments, symptoms are primary, while in other embodiments, symptoms are secondary. As used herein, “primary” refers to a symptom that is a direct result of infection with a pathogen or direct result of challenge with an antigen, while “secondary” refers to a symptom that is derived from or consequent to a primary cause.

In certain embodiments, the invention provides methods of treating a subject suffering from bronchitis, comprising the step of administering to a subject a compound comprising a lipid or phospholipid moiety bond to a physiologically acceptable monomer, dimer, oligomer, or polymer, and/or a pharmaceutically acceptable salt or a pharmaceutical product thereof, in an amount effective to treat the subject suffering from bronchitis. In some embodiments, the invention provides methods of treating a subject suffering from bronchitis, comprising the step of administering to a subject any one of the compounds according to the invention, in an amount effective to treat the subject suffering from bronchitis.

In certain embodiments, the invention provides methods of treating a subject suffering from an obstructive respiratory disease, comprising the step of administering to a subject a compound comprising a lipid or phospholipid moiety bond to a physiologically acceptable monomer, dimer, oligomer, or polymer, and/or a pharmaceutically acceptable salt or a pharmaceutical product thereof, in an amount effective to treat the subject suffering from an obstructive respiratory disease. In some embodiments, the invention provides methods of treating a subject suffering from an obstructive respiratory disease, comprising the step of administering to a subject any one of the compounds according to the invention, in an amount effective to treat the subject suffering from an obstructive respiratory disease. In another embodiment, the obstructive respiratory disease is asthma.

In certain embodiments of the present invention, the physiologically acceptable monomer is either a salicylate, salicylic acid, aspirin, a monosaccharide, lactobionic acid, maltose, an amino acid, glycine, carboxylic acid, acetic acid, butyric acid, dicarboxylic acid, glutaric acid, succinic acid, fatty acid, dodecanoic acid, didodecanoic acid, bile acid, cholic acid, cholesterylhemmisuccinate; or wherein the physiologically acceptable dimer or oligomer is a dipeptide, a disaccharide, a trisaccharide, an oligopeptide, or a di- or trisaccharide monomer unit of heparin, heparan sulfate, keratin, keratan sulfate, chondroitin, chondroitin sulfate, dermatin, dermatan sulfate, dextran, or hyaluronic acid; or wherein the physiologically acceptable polymer is a glycosaminoglycan, polygelin (‘hemaccell’), alginate, hydroxyethyl starch (hetastarch), polyethylene glycol, polycarboxylated polyethylene glycol, chondroitin sulfate, keratin, keratin sulfate, heparan sulfate, dermatin, dermatan sulfate, carboxymethylcellulose, heparin, dextran, or hyaluronic acid. In some embodiments, the physiologically acceptable polymer is chondroitin sulfate. In some embodiments, the chondroitin sulfate is chondroitin-6-sulfate, chondroitin-4-sulfate or a derivative thereof. In some embodiments, the physiologically acceptable polymer is hyaluronic acid.

In certain embodiments of the invention, the lipid or phospholipid moiety is either phosphatidic acid, an acyl glycerol, monoacylglycerol, diacylglycerol, triacylglycerol, sphingosine, sphingomyelin, chondroitin-4-sulphate, chondroitin-6-sulphate, ceramide, phosphatidylethanolamine, phosphatidylserine, phosphatidylcholine, phosphatidylinositol, or phosphatidylglycerol, or an ether or alkyl phospholipid derivative thereof, and the physiologically acceptable monomer or polymer moiety is either aspirin, lactobionic acid, maltose, glutaric acid, polyethylene glycol, carboxymethylcellulose, heparin, dextran, hemacell, hetastarch, or hyaluronic acid. In some embodiments, the phospholipid moiety is phosphatidylethanolamine.

In certain embodiments, obstructive respiratory disease is a disease of luminal passages in the lungs, marked by dyspnea, tachypnea, or ausculatory or radiological signs of airway obstruction. Obstructive respiratory disease comprises asthma, acute pulmonary infections, acute respiratory distress syndrome, chronic obstructive pulmonary disease, bronchitis, rhinitis, and allergic rhinitis. In some embodiments, the pathophysiology is attributed to obstruction of air flow due to constriction of airway lumen smooth muscle and accumulation of infiltrates in and around the airway lumen.

In certain embodiments, asthma is a disease process wherein the bronchi may be narrowed, making breathing difficult. In some embodiments, symptoms comprise wheezing, difficulty breathing (particularly exhaling air), tightness in the chest, or a combination thereof. In some embodiments, factors which can exacerbate asthma include rapid changes in temperature or humidity, allergies, upper respiratory infections, exercise, stress, smoke (e.g., cigarette), or a combination thereof. Such asthma may be allergic asthma, or allergic bronchitis.

In certain embodiments, rhinitis comprises an inflammation of the mucous membrane of the nose. In some embodiments, allergic rhinitis is an inflammatory response in the nasal passages to an allergic stimulus. In certain embodiments, symptoms comprise nasal congestion, sneezing, runny, itchy nose, or a combination thereof.

In certain embodiments, chronic obstructive pulmonary disease is a progressive disease process that most commonly results from smoking. In some embodiments, chronic obstructive pulmonary disease comprises difficulty breathing, wheezing, coughing, which may be a chronic cough, or a combination thereof. In some embodiments, chronic obstructive pulmonary disease may lead to health complications, which in certain embodiments, may comprise bronchitis (e.g., allergic bronchitis), pneumonia, lung cancer, or a combination thereof.

Cellular elaboration of cytokines and chemokines serve an important regulatory function in health: however, when a hyperactive response to stress or disease is triggered, these compounds may present in excess and damage tissue, thereby pushing the disease state toward further deterioration. Cytokine overproduction is involved in numerous diseases, such as sepsis, airway and lung injury, renal failure, transplant rejection, skin injuries, intestine injuries, cancer development and metastasis, central nervous system disorders, vaginal bacterial infection, and more.

In certain embodiments, the present invention offers methods for the treatment of disease based upon administration of lipids covalently conjugated through their polar head group to a physiologically acceptable chemical moiety, which may be of high or low molecular weight.

In some embodiments, the lipid compounds (Lipid-conjugates) of the present invention are described by the general formula:

[phosphatidylethanolamine-Y]n-X

[phosphatidylserine-Y]n-X

[phosphatidylcholine-Y]n-X

[phosphatidylinositol-Y]n-X

[phosphatidylglycerol-Y]n-X

[phosphatidic acid-Y]n-X

[lyso-phospholipid-Y]n-X

[diacyl-glycerol-Y]n-X

[monoacyl-glycerol-Y]n-X

[sphingomyelin-Y]n-X

[sphingosine-Y]n-X

[ceramide-Y]n-X
wherein
Y is either nothing or a spacer group ranging in length from 2 to 30 atoms; and
X is a physiologically acceptable monomer, dimer, oligomer or polymer, and
n, the number of lipid molecules bound to X, is a number from 1 to 1000.

In one embodiment of this invention, n is a number from 1 to 1000. In another embodiment, n is a number from 1 to 500. In another embodiment, n is a number from 2 to 500. In another embodiment, n is a number from 2 to 1000. In another embodiment, n is a number from 1 to 100. In another embodiment, n is a number from 100 to 300. In another embodiment, n is a number from 300 to 500. In another embodiment, n is a number from 500 to 800.

In one embodiment, the lipid compounds of this invention, known herein as lipid conjugates (Lipid-conjugates) are now disclosed to possess a combination of multiple and potent pharmacological effects in addition to the ability to inhibit the extracellular form of the enzyme phospholipase A2. The set of compounds comprising phosphatidylethanolamine covalently bound to a physiologically acceptable monomer or polymer is referred to herein as the PE-conjugates. Related derivatives, in which either phosphatidylserine, phosphatidylcholine, phosphatidylinositol, phosphatidic acid or phosphatidylglycerol are employed in lieu of phosphatidylethanolamine as the lipid moiety provide equivalent therapeutic results, based upon the biological experiments described below for the Lipid-conjugates and the structural similarities shared by these compounds. Other Lipid-conjugate derivatives relevant to this invention are Lipid-conjugates wherein at least one of the fatty acid groups of the lipid moieties at position C1 or C2 of the glycerol backbone are substituted by a long chain alkyl group attached in either ether or alkyl bonds, rather than ester linkage.

As defined by the structural formulae provided herein for the Lipid-conjugates, these compounds may contain between one to one thousand lipid moieties bound to a single physiologically acceptable polymer molecule.

Administration of the Lipid-conjugates in a diversity of animal and cell models of disease invokes remarkable, and unexpected, cytoprotective effects, which are useful in the treatment of disease. They are able to stabilize biological membranes; inhibit cell proliferation; suppress free radical production; suppress nitric oxide production; reduce cell migration across biological barriers; influence chemokine levels, including MCP-1, ENA-78, Gro α, and CX3C; affect gene transcription and modify the expression of MHC antigens; bind directly to cell membranes and change the water structure at the cell surface; inhibit the uptake of oxidized lipoprotein; prevent airway smooth muscle constriction; suppress neurotransmitter release: reduce expression of tumor necrosis factor-α (TNF-α); modify expression of transcription factors such as NFκB; inhibit extracellular degradative enzymes, including collagenase, heparinase, hyaluronidase, in addition to that of PLA2; and inhibit viral infection of white cells. Thus the Lipid-conjugatesprovide far-reaching cytoprotective effects to an organism suffering from a disease wherein one or more of the presiding pathophysiological mechanisms of tissue damage entails either oxidation insult giving rise to membrane fragility; hyperproliferation behavior of cells giving rise to stenotic plaque formation in vascular tissue, angiogenesis and benign or malignant cancer disease, or psoriasis; aberrant cell migration giving rise to brain injury or tumor cell metastases; excessive expression of chemokines and cytokines associated with central nervous system (CNS) insult, sepsis, ARDS, or immunological disease; cell membrane damage giving rise to CNS insult, CVS disease, or hemolysis: peroxidation of blood proteins and cell membranes giving rise to atherosclerosis or reperfusion injury; excessive nitric oxide production giving rise to CNS insult, reperfusion injury, and septic shock; interaction with major histocompatability antigens (MHC) associated with autoimmune diseases and alloimmune syndromes, such as transplant rejection.

In certain embodiments of the present invention, the useful pharmacological properties of the lipid or Lipid-conjugates may be applied for clinical use, and disclosed herein as methods for treatment of a disease. The biological basis of these methods may be readily demonstrated by standard cellular and animal models of disease as described below.

While pharmacological activity of the Lipid-conjugates described herein may be due in part to the nature of the lipid moiety, the multiple and diverse combination of pharmacological properties observed for the Lipid-conjugates emerges from the ability of the compound structure to act essentially as several different drugs in one chemical entity. Thus, for example, internal mucosal injury, as may occur in colitis or Crohn's disease, may be attenuated by any one or all of the pharmaceutical activities of immune suppression, anti-inflammation, anti-oxidation, nitric oxide production, or membrane stabilization. Protection of blood vessels from periluminal damage, as may occur in atherosclerosis, may entail activity from anti-proliferative, anti-chemokine, antioxidant, or antimigratory effects. Treatment or prevention of bronchitis or obstructive respiratory disease may involve any one of the many activities of the Lipid-conjugates ranging from suppression of nitric oxide, anti-chemokine, anti-proliferative, or membrane stabilization effects.

The use of a single chemical entity with potent anti-oxidant, membrane-stabilizing, anti-proliferative, anti-chemokine, anti-migratory, and anti-inflammatory activity provides increased cytoprotection relative to the use of several different agents each with a singular activity. The use of a single agent having multiple activities over a combination or plurality of different agents provides uniform delivery of an active molecule, thereby simplifying issues of drug metabolism, toxicity and delivery. The compounds of the present invention also exhibit properties present only in the combined molecule, not in the individual components.

In certain embodiments, the compounds of the invention may be used for acute treatment of temporary conditions, or may be administered chronically, especially in the case of progressive, recurrent, or degenerative disease. In one embodiment of the invention, the concentrations of the compounds will depend on various factors, including the nature of the condition to be treated, the condition of the patient, the route of administration and the individual tolerability of the compositions.

In another embodiment, the invention provides low-molecular weight Lipid-conjugates, previously undisclosed and unknown to possess pharmacological activity, of the general formula:

[Phosphatidylethanolamine-Y]n-X

[Phosphatidylserine-Y]n-X

[Phosphatidylcholine-Y]n-X

[Phosphatidylinositol-Y]n-X

[Phosphatidylglycerol-Y]n-X

[Phosphatidic acid-Y]n-X

[lyso-phospholipid-Y]n-X

[diacyl-glycerol-Y]n-X

[monoacyl-glycerol-Y]n-X

[sphingomyelin-Y]n-X

[sphingosine-Y]n-X

[ceramide-Y]n-X
wherein
Y is either nothing or a spacer group ranging in length from 2 to 30 atoms; and
X is salicylate, salicylic acid, aspirin, a monosaccharide, lactobionic acid, maltose, an amino acid, glycine, carboxylic acid, acetic acid, butyric acid, dicarboxylic acid, glutaric acid, succinic acid, fatty acid, dodecanoic acid, didodecanoic acid, bile acid, cholic acid, cholesterylhemmisuccinate, a dipeptide, a disaccharide, a trisaccharide, an oligosaccharide, an oligopeptide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, or hyaluronic acid, a glycosaminoglycan, polygeline (‘haemaccel’), alginate, hydroxyethyl starch (hetastarch), polyethylene glycol, polycarboxylated polyethylene glycol, chondroitin-6-sulfate, chondroitin-4-sulfate, keratin, keratin sulfate, heparan sulfate, dermatin, dermatan sulfate, carboxymethylcellulose, heparin, dextran, or hyaluronic acid; and n, the number of lipid molecules bound to X, is a number from 1 to 1000.

In certain embodiments of this invention, n is a number from 1 to 1000. In some embodiments, n is a number from 1 to 500. In other embodiments, n is a number from 1 to 100. In yet other embodiments, n is a number from 100 to 300. In further embodiments, n is a number from 300 to 500. In yet further embodiments, n is a number from 500 to 800.

In certain embodiments of the invention, these Lipid-conjugate derivatives possess wide-spectrum pharmacological activity and, as pharmaceutical agents administered to treat disease, are considered analogous to the Lipid-conjugates comprised from high molecular weight polymers. Other lipid-conjugate derivatives relevant to this invention are glycerolipid moieties in which at least one of the two long chain alkyl groups in position C1 and C2 of the glycerol backbone are attached in ether or alkyl bonds, rather than ester linkage.

The present invention is further illustrated in the following examples of the therapeutic Lipid-conjugate compounds, their chemical preparation, their anti-disease activity, and methods of use as pharmaceutical compositions in the treatment of disease.

### Compounds

In the methods, according to embodiments of the invention, the Lipid-conjugates administered to the subject are comprised from at least one lipid moiety covalently bound through an atom of the polar head group to a monomer or polymeric moiety (referred to herein as the conjugated moiety) of either low or high molecular weight. When desired, an optional bridging moiety can be used to link the Lipid-conjugates moiety to the monomer or polymeric moiety. The conjugated moiety may be a low molecular weight carboxylic acid, dicarboxylic acid, fatty acid, dicarboxylic fatty acid, acetyl salicylic acid, cholic acid, cholesterylhemisuccinate, or mono- or di-saccharide, an amino acid or dipeptide, an oligopeptide, a glycoprotein mixture, a di- or trisaccharide monomer unit of a glycosaminoglycan such as a repeating unit of heparin, heparan sulfate, hyaluronic acid, chondroitin-sulfate, dermatan, keratan sulfate, or a higher molecular weight peptide or oligopeptide, a polysaccharide, polyglycan, protein, glycosaminoglycan, or a glycoprotein mixture. From a composition aspect, phospholipid-conjugates of high molecular weight, and associated analogues, are the subject of U.S. Pat. No. 5,064,817, as well as the publications cited herein.

In certain embodiments of the invention, when the conjugated carrier moiety is a polymer, the ratio of lipid moieties covalently bound may range from one to one thousand lipid residues per polymer molecule, depending upon the nature of the polymer and the reaction conditions employed. For example, the relative quantities of the starting materials, or the extent of the reaction time, may be modified in order to obtain Lipid-conjugate products with either high or low ratios of lipid residues per polymer, as desired.

The term “moiety” means a chemical entity otherwise corresponding to a chemical compound, which has a valence satisfied by a covalent bond.

Examples of polymers which can be employed as the conjugated moiety for producing Lipid-conjugates for use in the methods of this invention may be physiologically acceptable polymers, including water-dispersible or -soluble polymers of various molecular weights and diverse chemical types, mainly natural and synthetic polymers, such as glycosaminoglycans, hyaluronic acid, heparin, heparin sulfate, chondroitin sulfate, chondroitin-6-sulfate, chondroitin-4-sulfate, keratin sulfate, dermatin, sulfate, plasma expanders, including polygeline (“Haemaccel”, degraded gelatin polypeptide crosslinked via urea bridges, produced by “Behring”), “hydroxyethylstarch” (Htastarch, HES) and extrans, food and drug additives, soluble cellulose derivatives (e.g., methylcellulose, carboxymethylcellulose), polyaminoacids, hydrocarbon polymers (e.g., polyethylene), polystyrenes, polyesters, polyamides, polyethylene oxides (e.g., polyethyleneglycols, polycarboxyethyleneglycol), polyvinnylpyrrolidones, polysaccharides, alginates, assimilable gums (e.g., xanthan gum), peptides, injectable blood proteins (e.g., serum albumin), cyclodextrin, and derivatives thereof.

Examples of monomers, dimers, and oligomers which can be employed as the conjugated moiety for producing Lipid-conjugates for use in the methods of the invention may be mono- or disaccharides, carboxylic acid, dicarboxylic acid, fatty acid, dicarboxylic fatty acid, acetyl salicylic acid, cholic acid, cholesterylhemisuccinate, and di- and trisaccharide unit monomers of glycosaminoglycans including heparin, heparan sulfate, hyaluronic acid, chondroitin, chondroitin-6-sulfate, chondroitin-4-sulfate, dermatin, dermatan sulfate, keratin, keratan sulfate, or dextran.

In some cases, according to embodiments of the invention, the monomer or polymer chosen for preparation of the Lipid-conjugate may in itself have select biological properties. For example, both heparin and hyaluronic acid are materials with known physiological functions. In the present invention, however, the Lipid-conjugates formed from these substances as starting materials display a new and wider set of pharmaceutical activities than would be predicted from administration of either heparin or hyaluronic acid which have not been bound by covalent linkage to a phospholipid. It can be shown, by standard comparative experiments as described below, that phosphatidylethanolamine (PE) linked to carboxymethylcellulose (referred to as CMPE, CMC-Peor CME), to hyaluronic acid (referred to as HYPE, HyPE, and Hyal-PE), to heparin (referred to as HEPPE, HepPE, HePPE, Hepa-PE), to chondroitine sulfate A (referred to as CSAPE, CsaPE, CsAPE), to Polygeline (haemaccel) (referred to HemPE, HEMPE), or to hydroxyethylstarch (referred to as HesPE, HESPE), are far superior in terms of potency and range of useful pharmaceutical activity to the free conjugates (the polymers above and the like). In fact, these latter substances are, in general, not considered useful in methods for treatment of most of the diseases described herein, and for those particular cases wherein their use is medically prescribed, such as ischemic vascular disease, the concentrations for their use as drugs are are several orders of magnitude higher. Thus, the combination of a phospholipid such as phosphatidylethanolamine, or related phospholipids which differ with regard to the polar head group, such as phosphatidylserine (PS), phosphatidylcholine (PC), phosphatidylinositol (PI), and phosphatidylglycerol (PG), results in the formation of a compound which has novel pharmacological properties when compared to the starting materials alone.

The biologically active lipid conjugates described herein can have a wide range of molecular weight, e.g., above 50,000 (up to a few hundred thousands) when it is desirable to retain the Lipid conjugate in the vascular system and below 50,000 when targeting to extravascular systems is desirable. The sole limitation on the molecular weight and the chemical structure of the conjugated moiety is that it does not result in a Lipid-conjugate devoid of the desired biological activity, or lead to chemical or physiological instability to the extent that the Lipid-conjugate is rendered useless as a drug in the method of use described herein.

In one embodiment, a compound according to embodiments of the invention is represented by the structure of the general formula (A):

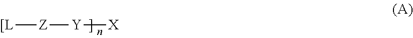

wherein
L is a lipid or a phospholipid;
Z is either nothing, ethanolamine, serine, inositol, choline, or glycerol;
Y is either nothing or a spacer group ranging in length from 2 to 30 atoms:
X is a physiologically acceptable monomer, dimer, oligomer, or polymer, wherein X is a glycosaminoglycan; and
n is a number from 1 to 1000;
wherein any bond between L, Z, Y and X is either an amide or an esteric bond.

In certain embodiments, a compound according to embodiments of the invention is represented by the structure of the general formula (I):

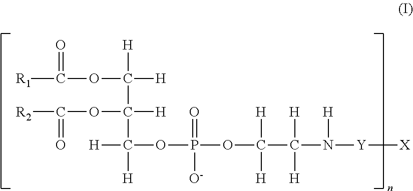

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms; and
X is either a physiologically acceptable monomer, dimer, oligomer or a physiologically acceptable polymer, wherein X is a glycosaminoglycan; and
n is a number from 1 to 1,000;
wherein if Y is nothing the phosphatidylethanolamine is directly linked to X via an amide bond and if Y is a spacer, the spacer is directly linked to X via an amide or an esteric bond and to the phosphatidylethanolamine via an amide bond.

Preferred compounds for use in the methods of the invention comprise one of the following as the conjugated moiety X: acetate, butyrate, glutarate, succinate, dodecanoate, didodecanoate, maltose, lactobionic acid, dextran, alginate, aspirin, cholate, cholesterylhemisuccinate, carboxymethyl-cellulose, heparin, hyaluronic acid, polygeline (haemaccel), polyethyleneglycol, and polycarboxylated polyethylene glycol. The polymers used as starting material to prepare the PE-conjugates may vary in molecular weight from 1 to 2,000 kDa.

Examples of phosphatidylethanolamine (PE) moieties are analogues of the phospholipid in which the chain length of the two fatty acid groups attached to the glycerol backbone of the phospholipid varies from 2-30 carbon atoms length, and in which these fatty acids chains contain saturated and/or unsaturated carbon atoms. In lieu of fatty acid chains, alkyl chains attached directly or via an ether linkage to the glycerol backbone of the phospholipid are included as analogues of PE. According to the present invention, a most preferred PE moiety is dipalmitoylphosphatidy-ethanolamine.

Phosphatidyl-ethanolamine and its analogues may be from various sources, including natural, synthetic, and semisynthetic derivatives and their isomers.

Phospholipids which can be employed in lieu of the PE moiety are N-methyl-PE derivatives and their analogues, linked through the amino group of the N-methyl-PE by a covalent bond; N,N-dimethyl-PE derivatives and their analogues linked through the amino group of the N,N-dimethyl-PE by a covalent bond, phosphatidylserine (PS) and its analogues, such as palmitoyl-stearoyl-PS, natural PS from various sources, semisynthetic PSs, synthetic, natural and artifactual PSs and their isomers. Other phospholipids useful as conjugated moieties in embodiments of this invention are phosphatidylcholine (PC), phosphatidylinositol (PI), phosphatidic acid and phosphoatidylglycerol (PG), as well as derivatives thereof comprising either phospholipids, lysophospholipids, phosphatidyic acid, sphingomyelins, lysosphingomyelins, ceramide, and sphingosine.

For PE-conjugates and PS-conjugates, the phospholipid is linked to the conjugated monomer or polymer moiety through the nitrogen atom of the phospholipid polar head group, either directly or via a spacer group. For PC, PI, and PG conjugates, the phospholipid is linked to the conjugated monomer or polymer moiety through either the nitrogen or one of the oxygen atoms of the polar head group, either directly or via a spacer group.

In other embodiments, a compound according to embodiments of the invention is to represented by the structure of the general formula (II):

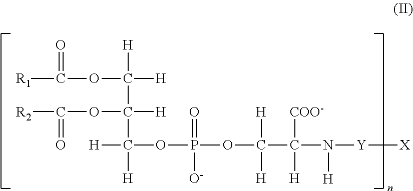

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a physiologically acceptable monomer, dimer, oligomer or polymer wherein x is a glycosaminoglycan; and
n is a number from 1 to 1000;
wherein if Y is nothing the phosphatidylserine is directly linked to X via an amide bond and if Y is a spacer, the spacer is directly linked to X via an amide or an esteric bond and to the phosphatidylserine via an amide bond.
In certain embodiments, the compound according to the invention be [phosphatidylserine-Y]n-X, wherein Y is either nothing or a spacer group ranging in length from 2 to 30 atoms, X is a physiologically acceptable monomer, dimer, oligomer or polymer wherein x is a glycosaminoglycan, and n is a number from 1 to 1000, wherein the phosphatidylserine may be bonded to Y or to X, if Y is nothing, via the COO⁻ moiety of the phosphatidylserine.

In further embodiments, a compound according to embodiments of the invention is represented by the structure of the general formula (ITT):

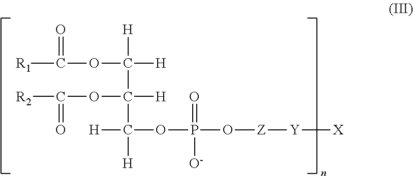

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phosphatidyl, Z, Y and X is either an amide or anesteric bond.

In yet other embodiments, a compound according to embodiments of the invention is represented by the structure of the general formula (IV)

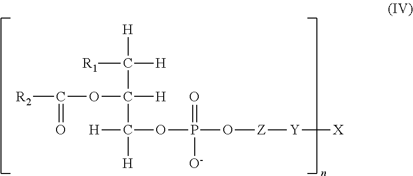

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In certain embodiments, a compound according to embodiments of the invention is represented by the structure of the general formula (V):

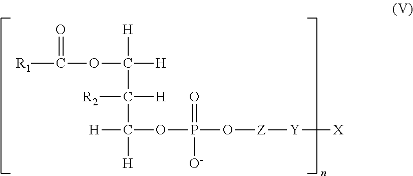

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In some embodiments, a compound according to embodiments of the invention is represented by the structure of the general formula (VI):

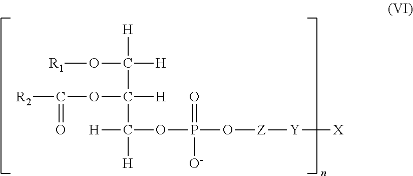

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In other embodiments, a compound according to embodiments of the invention is represented by the structure of the general formula (VII):

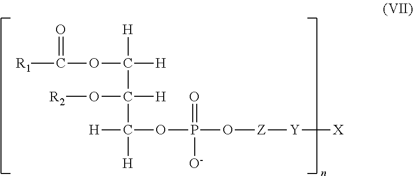

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In some embodiments of the invention, phosphatidylcholine (PC), Phosphatidylinositol (PI), phosphatidic acid (PA), wherein Z is nothing, and Phosphatidylglycerol (PG) conjugates are herein defined as compounds of the general formula (III).

In certain embodiments of the invention Y is nothing. Non limiting examples of suitable divalent groups forming the optional bridging group (spacer) Y, according to embodiments of the invention, are straight or branched chain alkylene, e.g., of 2 or more, preferably 4 to 30 carbon atoms, —CO-alkylene-CO, —NH-alkylene-NH—, —CO-alkylene-NH—, —NH-alkylene-NHCO-alkylene-NH—, an amino acid, cycloalkylene, wherein alkylene in each instance, is straight or branched chain and contains 2 or more, preferably 2 to 30 atoms in the chain, —(—O—CH(CH₃)CH₂—)ₓ— wherein x is an integer of 1 or more.

According to embodiments of the invention, in addition to the traditional phospholipid structure, related derivatives for use in this invention are phospholipids modified at the C1 or C2 position to contain an ether or alkyl bond instead of an ester bond. In some embodiments of the invention, the alkyl phospholipid derivatives and ether phospholipid derivatives are exemplified herein.

In still other embodiments, a compound according to embodiments of the invention is represented by the structure of the general formula (VIII):

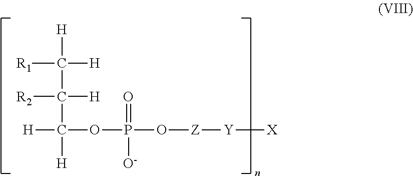

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In still further embodiments, a compound according to embodiments of the invention is to represented by the structure of the general formula (IX):

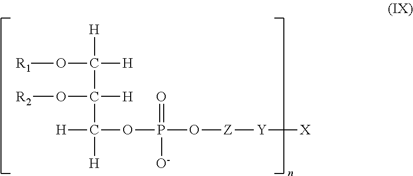

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In certain embodiments, a compound according to embodiments of the invention is represented by the structure of the general formula (IXa):

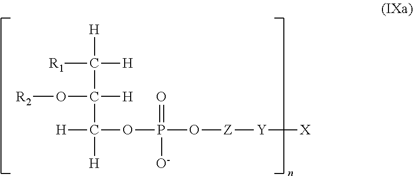

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In certain other embodiments, the a compound according to embodiments of the invention is represented by the structure of the general formula (IXb):

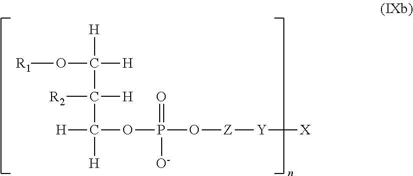

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms:
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond.

In further embodiments, a compound according to embodiments of the invention is represented by the structure of the general formula (X):

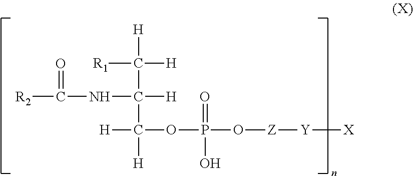

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the ceramide phosphoryl, Z, Y and X is either an amide or an esteric bond.

In still further embodiments, a compound according to embodiments of the invention is represented by the structure of the general formula (XI):

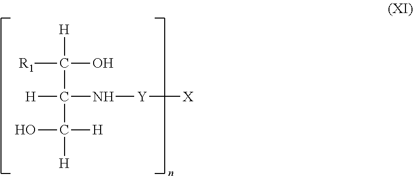

wherein
R₁ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein if Y is nothing the sphingosyl is directly linked to X via an amide bond and if Y is a spacer, the spacer is directly linked to X and to the sphingosyl via an amide bond and to X via an amide or an esteric bond.

In yet further embodiments, a compound according to embodiments of the invention is represented by the structure of the general formula (XII):

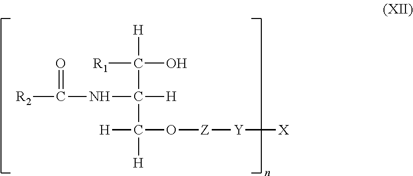

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
L is ceramide;
Z is either nothing, ethanolamine, serine, inositol, choline, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the ceramide, Z, Y and X is either an amide or an esteric bond.

In some embodiments, a compound according to embodiments of the invention is represented by the structure of the general formula (XIII):

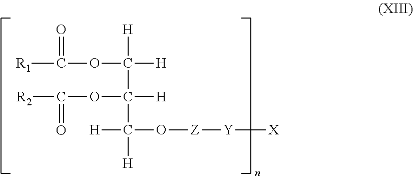

wherein

R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the diglyceryl, Z, Y and X is either an amide or an esteric bond.

In certain embodiments, a compound according to embodiments of the invention is represented by the structure of the general formula (XIV):

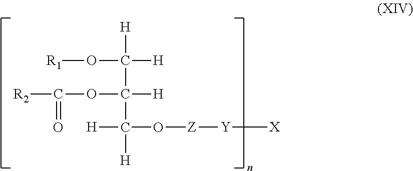

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms:
X is a physiologically acceptable monomer, dimer, oligomer or polymer, wherein x is a lycosaminoglycan; and
n is a number from 1 to 1000;
wherein any bond between the glycerolipid, Z, Y and X is either an amide or an esteric bond.

In additional embodiments, a compound according to embodiments of the invention is represented by the structure of the general formula (XV):

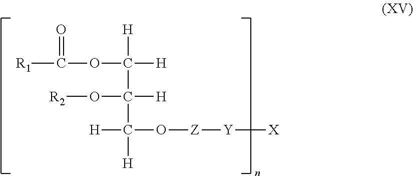

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms:
X is a physiologically acceptable monomer, dimer, oligomer or polymer, wherein x is a glycosaminoglycan; and
n is a number from 1 to 1000;
wherein any bond between the glycerolipid, Z, Y and X is either an amide or an esteric bond.

In other embodiments, a compound according to embodiments of the invention is represented by the structure of the general formula (XVI):

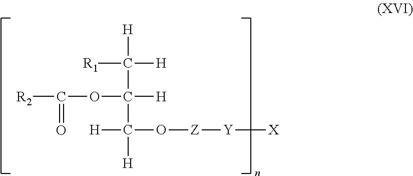

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond.

In yet other embodiments, a compound according to embodiments of the invention is represented by the structure of the general formula (XVII):

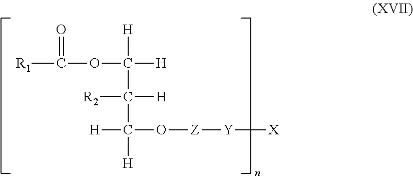

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond.

In still other embodiments, a compound according to embodiments of the invention is represented by the structure of the general formula (XVIII):

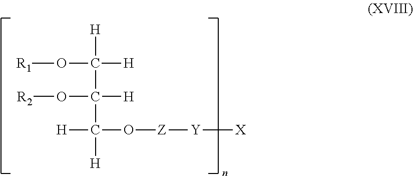

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond.

In further embodiments, a compound according to embodiments of the invention is represented by the structure of the general formula (XIX):

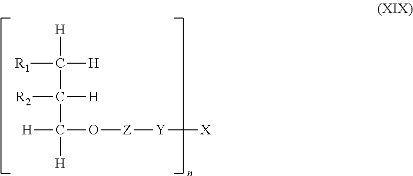

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond.

In yet further embodiments, a compound according to embodiments of the invention is represented by the structure of the general formula (XX):

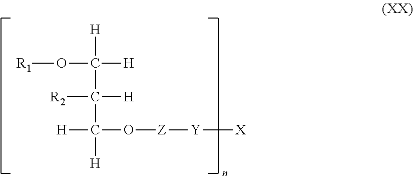

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms:
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond.

In yet still further embodiments, a compound according to embodiments of the invention is represented by the structure of the general formula (XXI):

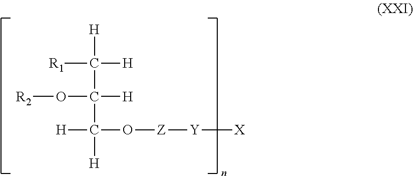

wherein
R₁ is either hydrogen or a linear, saturated, mono-unsaturated, or poly-unsaturated, alkyl chain ranging in length from 2 to 30 carbon atoms;

R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond.

In certain embodiments of the invention, the glycosaminoglycan may be, inter alia, hyaluronic acid, heparin, heparan sulfate, chondroitin sulfate, keratin, keratan sulfate, dermatan sulfate or a derivative thereof.

In some embodiments, the glycosaminoglycan is di- and trisaccharide unit monomers of glycosaminoglycans. In certain embodiments, the chondroitin sulfate may be, inter alia, chondroitin-6-sulfate, chondroitin-4-sulfate or a derivative thereof.

In certain embodiments of the invention, the sugar rings of the glycosaminoglycan are intact. In some embodiments, intact refers to closed. In other embodiments, intact refers to natural. In yet other embodiments, intact refers to unbroken.

In certain embodiments of the invention, the structure of the lipid or phospholipids in any compound according to the invention is intact. In some embodiments, the natural structure of the lipid or phospholipids in any compound according to the invention is maintained.

In some embodiments, the compounds according to the invention are biodegradable.

In certain embodiments, the compound according to the invention is a compound represented by the structure of the general formula (A):

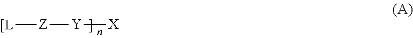

wherein
L is phosphatidyl;
Z is ethanolamine, wherein L and Z are chemically bonded resulting in phosphatidylethanolamine;
Y is nothing;
X is hyaluronic acid; and
n is a number from 1 to 1000;
wherein any bond between the phosphatidylethanolamine and the hyaluronic acid is an amide bond.

In some embodiments, the compound according to the invention is a compound represented by the structure of the general formula (A):

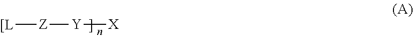

wherein
L is phosphatidyl;
Z is ethanolamine, wherein L and Z are chemically bonded resulting in phosphatidylethanolamine;
Y is nothing;
X is chondroitin sulfate; and
n is a number from 1 to 1000;
wherein any bond between the phosphatidylethanolamine and the chondroitin sulfate is an amide bond.

In certain embodiments, the invention provides methods of treating a subject suffering from asthma, comprising the step of administering to a subject any one of the compounds according to the invention, or any combination thereof, in an amount effective to treat the subject suffering from asthma. In some of these embodiments, the compounds according to the invention include, inter alia, the compounds represented by the structures of the general formulae: (A), (I), (II), (III), (IV), (V), (VI), (VII), (VIII), (IX), (IXa), (IXb), (X), (XI), (XII), (XIII), (XIV), (XV), (XVI), (XVII), (XVIII), (XIX), (XX), (XXI), (XXII) or any combination thereof. In other embodiments, the invention provides methods of preventing asthma in a subject.

In certain embodiments, the invention provides methods of treating a subject suffering from rhinitis, comprising the step of administering to a subject any one of the compounds according to the invention, or any combination thereof, in an amount effective to treat the subject suffering from rhinitis. In some of these embodiments, the compounds according to the invention include, inter alia, the compounds represented by the structures of the general formulae: (A), (I), (II), (III), (IV), (V), (VI), (VII), (VIII), (IX), (IXa), (IXb), (X), (XI), (XII), (XIII), (XIV), (XV), (XVII), (XVII), (XVIII), (XIX), (XX), (XXI), (XXII) or any combination thereof. In other embodiments, the invention provides methods of preventing rhinitis in a subject.

In certain embodiments, the invention provides methods of treating a subject suffering from bronchitis, comprising the step of administering to a subject any one of the compounds according to the invention, or any combination thereof, in an amount effective to treat the subject suffering from bronchitis. In some of these embodiments, the compounds according to the invention include, inter alia, the compounds represented by the structures of the general formulae: (A), (I), (II), (III), (IV), (V), (VI), (VII), (VIII), (IX), (IXa), (IXb), (X), (XI), (XII), (XIII), (XIV), (XV), (XVI), (XVII), (XVIII), (XIX), (XX), (XXI), (XXII) or any combination thereof. In other embodiments, the invention provides methods of preventing bronchitis in a subject.

In certain embodiments, the invention provides methods of treating a subject suffering from chronic obstructive pulmonary disease, comprising the step of administering to a subject any one of the compounds according to the invention, or any combination thereof, in an amount effective to treat the subject suffering from chronic obstructive pulmonary disease. In some of these embodiments, the compounds according to the invention include, inter alia, the compounds represented by the structures of the general formulae: (A), (I), (II), (III), (IV), (V), (VI), (VII), (VIII), (IX), (IXa), (IXb), (X), (XI), (XII), (XIII), (XIV), (XV), (XVI), (XVII), (XVIII), (XIX), (XX), (XXI), (XXII) or any combination thereof. In other embodiments, the invention provides methods of preventing chronic obstructive pulmonary disease in a subject.

In certain embodiments, the invention provides methods of treating a subject suffering from an obstructive respiratory disease, comprising the step of administering to a subject any one of the compounds according to the invention, or any combination thereof, in an amount effective to treat the subject suffering from an obstructive respiratory disease. In another embodiment, the compounds according to the invention include, inter alia, the compounds represented by the structures of the general formulae: (A), (I), (II), (II), (IV), (V), (VI), (VII), (VIII), (IX), (IXa), (IXb), (X), (XI), (XII), (XIII), (XIV), (XV), (XVI), (XVII), (XVIII), (XIX), (XX), (XXI), (XXII) or any combination thereof. In some embodiments, the obstructive respiratory disease is asthma. In some embodiments, the obstructive respiratory disease is rhinitis. In some embodiments, the obstructive respiratory disease is bronchitis. In some embodiments, the obstructive respiratory disease is chronic obstructive pulmonary disease. In some embodiments, the invention provides methods of preventing asthma, bronchitis, rhinitis, allergic rhinitis, chronic obstructive pulmonary disease, obstructive respiratory disease, or a combination thereof, in a subject.

Illustrative of preferred Lipid-conjugates for use in the methods according to embodiments of this invention are those in which the lipid/phospholipid moiety is linked directly or indirectly through a bridging moiety listed below.

| phospho- | | | |
|---|---|---|---|
| lipid | spacer | polymer (m.w.) | abbreviation |
| PE | Dicarboxylic | Polygeline (haemaccel) | HeMPE; HemPE |
| | acid + | (4-40 kDa) | |
| | Diamine | | |
| PE | None | Carboxymethylcellulose | CMPE; CMC-PE |
| | | (20-500 kDa) | |
| PE | None | Hyaluronic acid | HYPE (HyPE) |
| | | (2-2000 kDa) | |
| PE | Dipalmitoic | Hyaluronic acid | HYPE- |
| | acid | (2-2000 kDa) | dipalmitoyl |
| PE | None | Polyethylene glycol | |
| PE | Y | Hydroxyethylstarch | HESPE; HesPE |
| PE | Dicarboxylic | Dextran | DexPE |
| | acid + | (1-2,000 kDa) | |
| | Diamine | | |
| PE | None | Dextran | DexPE |
| | | (1-2,000 kDa) | |
| PE | None | Albumin | |
| PE | None | Alginate | |
| | | (2-2000 kDa) | |
| PE | None | Polyaminoacid | |
| PE | None | Lactobionic acid | |
| PE | None | Acetylsalicylate | |
| PE | None | Cholesteryl- | |
| | | hemmisuccinate | |
| PE | None | Maltose | |
| PE | Y | None | Cholic acid |
| PE | None | Polycarboxylated | |
| | | polyethylene glycol | |
| PE | None | Heparin | HEPPE; HEPE; |
| | | (0.5-110 kDa) | HepPE |
| Dimyristoyl- | Y | Variable | DMPE |
| PE | | | |
| Dimyristoyl- | Y | Hyaluronic acid | HyDMPE |
| PE | | | |
| PS | Y | Polygeline (haemaccel) | |
| PS | Y | Heparin | |
| PS | Y | Hyaluronic acid | |
| PC | Y | Polygeline (haemaccel) | |
| PC | Y | Heparin | |
| PC | Y | Hyaluronic acid | |
| PI | Y | Polygeline (haemaccel) | |
| PI | Y | Heparin | |
| PI | Y | Hyaluronic acid | |
| PG | Y | Polygeline (haemaccel) | |
| PG | Y | Heparin | |
| PE | Y | Chondoitin sulfates | CSPE |
| PE | Y | Polygeline (haemaccel) | |
| PG | Y | Hyaluronic acid | |

In some embodiments of the invention, the compounds administered are HyPE, CSAPE, CMPE, HemPE, HesPE, DexPE and As-PE and pharmaceutically acceptable salts thereof, in combination with a physiologically acceptable carrier or solvent. These polymers, when chosen as the conjugated moiety, may vary in molecular weights from 200 to 2,000,000 Daltons. Various molecular weight species have been shown to have the desired biological efficacy, as shown in the section below.

In addition to the compounds of the Examples, further illustrative compounds of this invention are set forth in the section below.

### Novel Compounds

Low molecular weight Lipid-conjugates, in which the conjugated moiety (X) is a monomer such as a salicylate, a bile acid, or cholesterylhemmisuccinate, or a di- or trisaccaharide unit monomer of a polyglycosoaminoglycan such as heparin, heparan sulfate, chondroitin-6-sulfate, chondroitin-4-sulfate, hyaluronic acid, keratin, keratan sulfate, dermatin, or dermatan sulfate, have not been described before. According to embodiments of the invention, these new compounds display a similar biological activity profile as demonstrated below for the other Lipid-conjugates and have the general formula

[Phosphatidylethanolamine-Y]ₙ—X

[Phosphatidylserine-Y]ₙ—X

[Phosphatidylcholine-Y]ₙ—X

[Phosphatidylinositol-Y]ₙ—

[Phosphatidylglycerol-Y]ₙ—X

[Phosphatidic acid-Y]ₙ—X

[lyso-phospholipid-Y]ₙ—X

[diacyl-glycerol-Y]ₙ—

[monoacyl-glycerol-Y]ₙ—X

[sphingomyelin-Y]ₙ—X

[sphingosine-Y]ₙ—X

[ceramide-Y]ₙ—X
wherein
Y is either nothing or a spacer group ranging in length from 2 to 30 atoms;
X is a mono- or disaccharide, carboxylated disaccharide, mono- or dicarboxylic acids, a salicylate, salicylic acid, aspirin, lactobionic acid, maltose, an amino acid, glycine, acetic acid, butyric acid, dicarboxylic acid, glutaric acid, succinic acid, fatty acid, dodecanoic acid, didodecanoic acid, bile acid, cholic acid, cholesterylhemmisuccinate, a di- or tripeptide, an oligopeptide, a trisacharide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, hyaluronic acid or glycosaminoglycan; and
n is the number of lipid moiety molecules bound to a molecule of X wherein n is a number from 1 to 100.

In certain embodiments, the glycosaminoglycan is a polymer (X) of disaccharide units. In some embodiments, the number of the disaccharide units in the polymer is m. In other embodiments, m is a number from 2-10,000. In yet other embodiments, m is a number from 2-500. In still other embodiments, m is a number from 2-1000. In yet still other embodiments, m is a number from 50-500. In some embodiments, m is a number from 2-2000. In some other embodiments, m is a number from 500-2000. In further embodiments, m is a number from 1000-2000. In still further embodiments, m is a number from 2000-5000. In yet further embodiments, m is a number from 3000-7000. In yet still further embodiments, m is a number from 5000-10,000. In some embodiments, a disaccharide unit of a glycosaminoglycan may be bound to one lipid or phospholipid moiety. In certain embodiments, each disaccharide unit of the glycosaminoglycan may be bound to zero or one lipid or phospholipid moieties. In some embodiments, the lipid or phospholipid moieties are bound to the —COOH group of the disaccharide unit. In other embodiments, the bond between the lipid or phospholipid moiety and the disaccharide unit is an amide bond.

According to certain embodiments, this invention provides lipid-GAG conjugate or phospholipid-GAG conjugate of this invention, and methods of use thereof, wherein said conjugate represented by the structures of the general formulae (A), (I), (II), (III), (IV), (V), (VI), (VII), (VIII), (IX), (IXa), (IXb), (X), (Xa), (XI), (XII), (XIIa), (XIII), (XIV), (XV), (XVI), (XVII), (XVIII), (XIX), (XX), (XXI), and (XXII). In some embodiments, the average molecular weight of said GAG is between 5 kD to 90 kD. In some embodiments, the average molecular weight of said GAG is between 5 kD to 60 kD. In some embodiments, the average molecular weight of said GAG is between 5 kD to 40 kD. In some embodiments, the average molecular weight of said GAG is between 5 kD to 15 kD. In some embodiments, the average molecular weight of said GAG is between 5 kD to 20 kD. In some embodiments, the lipid-GAG conjugate is a phospholipid-GAG conjugate

In certain embodiments of this invention, low molecular weight phosphatidylethanolamine (PE)-conjugates are defined hereinabove as the compounds of formula (I) wherein:
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a mono- or disaccharide, carboxylated disaccharide, mono- or dicarboxylic acids, a salicylate, salicylic acid, aspirin, lactobionic acid, maltose, an amino acid, glycine, acetic acid, butyric acid, dicarboxylic acid, glutaric acid, succinic acid, fatty acid, dodecanoic acid, didodecanoic acid, bile acid, cholic acid, cholesterylhemmisuccinate, a di- or tripeptide, an oligopeptide, a trisacharide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, hyaluronic acid or glycosaminoglycan; and
n is the number of lipid moity molecules bound to a molecule of X wherein n is a number from 1 to 1000.

In some embodiments, the molecular weight of said glycosaminoglycan is between 5 kD and 20 kD. In other embodiments, n is a number between 1 to 100. In yet other embodiments, said glycosaminoglycan is between 5 kD and 20 kD and n is between 1 to 100.

In certain embodiments of this invention, low molecular weight phosphatidylserine (PS)-conjugates are defined hereinabove as the compounds of formula (II) wherein:
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a mono- or disaccharide, carboxylated disaccharide, mono- or dicarboxylic acids, a salicylate, salicylic acid, aspirin, lactobionic acid, maltose, an amino acid, glycine, acetic acid, butyric acid, dicarboxylic acid, glutaric acid, succinic acid, fatty acid, dodecanoic acid, didodecanoic acid, bile acid, cholic acid, cholesterylhemmisuccinate, a di- or tripeptide, an oligopeptide, a trisaccharide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, hyaluronic acid or glycosaminoglycan; and
n is the number of lipid moiety molecules bound to a molecule of X wherein n is a number from 1 to 1000.

In some embodiments, the molecular weight of said glycosaminoglycan is between 5 kD and 20 kD. In other embodiments, n is a number between 1 to 100. In yet other embodiments, said glycosaminoglycan is between 5 kD and 20 kD and n is between 1 to 100.

In certain embodiments of this invention, Phosphatidylcholine (PC), Phosphatidylinositol (PI), and Phosphatidylglycerol (PG) conjugates are hereinabove defined as the compounds of formula (III) wherein:
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a mono- or disaccharide, carboxylated disaccharide, mono- or dicarboxylic acids, a salicylate, salicylic acid, aspirin, lactobionic acid, maltose, an amino acid, glycine, acetic acid, butyric acid, dicarboxylic acid, glutaric acid, succinic acid, fatty acid, dodecanoic acid, didodecanoic acid, bile acid, cholic acid, cholesterylhemmisuccinate, a di- or tripeptide, an oligopeptide, a trisaccharide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, hyaluronic acid or glycosaminoglycan; and
n is the number of lipid moiety molecules bound to a molecule of X wherein n is a number from 1 to 1000.

In some embodiments, the molecular weight of said glycosaminoglycan is between 5 kD and 20 kD. In other embodiments, n is a number between 1 to 100. In yet other embodiments, said glycosaminoglycan is between 5 kD and 20 kD and n is between 1 to 100.

Examples of suitable divalent groups forming the optional bridging group Y are straight- or branched-chain alkylene, e.g., of 2 or more, preferably 4 to 18 carbon atoms, —CO-alkylene-CO, —NH-alkylene—NH—, —CO-alkylene-NH—, cycloalkylene, wherein alkylene in each instance, is straight or branched chain and contains 2 or more, preferably 2 to 18 carbon atoms in the chain, —(—O—CH(CH₃)CH₂—)ₓ— wherein x is an integer of 1 or more.

In some embodiments, in addition to the traditional phospholipid structure, related derivatives for use in this invention are phospholipids modified at the C1 or C2 position to contain an ether or alkyl bond instead of an ester bond. These derivatives are exemplified hereinabove by the general formulae (VIII) and (IX) wherein:
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a mono- or disaccharide, carboxylated disaccharide, mono- or dicarboxylic acids, a salicylate, salicylic acid, aspirin, lactobionic acid, maltose, an amino acid, glycine, acetic acid, butyric acid, dicarboxylic acid, glutaric acid, succinic acid, fatty acid, dodecanoic acid, didodecanoic acid, bile acid, cholic acid, cholesterylhemmisuccinate, a di- or tripeptide, an oligopeptide, a trisaccharide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, hyaluronic acid or glycosaminoglycan; and
n is the number of lipid moiety molecules bound to a molecule of X wherein n is a number from 1 to 1000.

In some embodiments, the molecular weight of said glycosaminoglycan is between 5 kD and 20 kD. In other embodiments, n is a number between 1 to 100. In yet other embodiments, said glycosaminoglycan is between 5 kD and 20 kD and n is between 1 to 100.

In some embodiments, related low molecular weight derivatives for use in this invention are exemplified hereinabove by the general formulae (X), (XI) and (XII) wherein:
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms:
X is a mono- or disaccharide, carboxylated disaccharide, mono- or dicarboxylic acids, a salicylate, salicylic acid, aspirin, lactobionic acid, maltose, an amino acid, glycine, acetic acid, butyric acid, dicarboxylic acid, glutaric acid, succinic acid, fatty acid, dodecanoic acid, didodecanoic acid, bile acid, cholic acid, cholesterylhemmisuccinate, a di- or tripeptide, an oligopeptide, a trisaccharide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, hyaluronic acid or glycosaminoglycan; and
In is the number of lipid moiety molecules bound to a molecule of X wherein n is a number from 1 to 1000.

In some embodiments, the molecular weight of said glycosaminoglycan is between 5 kD and 20 kD. In other embodiments, n is a number between 1 to 100. In yet other embodiments, said glycosaminoglycan is between 5 kD and 20 kD and n is between 1 to 100.

In some embodiments, related low molecular weight derivatives for use in this invention are exemplified hereinabove by the general formulae (XIll) wherein:
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a mono- or disaccharide, carboxylated disaccharide, mono- or dicarboxylic acids, a salicylate, salicylic acid, aspirin, lactobionic acid, maltose, an amino acid, glycine, acetic acid, butyric acid, dicarboxylic acid, glutaric acid, succinic acid, fatty acid, dodecanoic acid, didodecanoic acid, bile acid, cholic acid, cholesterylhemmisuccinate, a di- or tripeptide, an oligopeptide, a trisaccharide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, hyaluronic acid or glycosaminoglycan; and
n is the number of lipid moiety molecules bound to a molecule of X wherein n is a number from 1 to 1000.

In some embodiments, the molecular weight of said glycosaminoglycan is between 5 kD and 20 kD. In other embodiments, n is a number between 1 to 100. In yet other embodiments, said glycosaminoglycan is between 5 kD and 20 kD and n is between 1 to 100.

In certain embodiments, related low molecular weight derivatives according to the invention may be exemplified herein by any of the general formulae (A), (I)-(XXI) wherein:

In certain embodimentsof the invention, X is covalently conjugated to a lipid. In some embodiments, x is covalently conjugated to a lipid via an amide bond. In other embodiments, x is covalently conjugated to a lipid via an esteric bond. In some embodiments, the lipid is phosphatidylethanolamine. In some embodiments, the GAG may be, inter alia, chondroitin sulfate. In certain embodiments, the conjugate is biodegradable. In some embodiments, the glycosaminoglycan is between 5 kD and 20 kD.

In some embodiments, the invention provides glycosaminoglycans (GAG) compound covalently conjugated to a lipid to obtain a compound having preferred therapeutic properties. In some embodiments, the GAG compound is covalently conjugated to a lipid via an amide bond. In some embodiments, the GAG compound is covalently conjugated to a lipid via an esteric bond. In some embodiments, the lipid may be, inter alia, phosphatidylethanolamine. In some embodiments, the GAG may be, inter alia, chondroitin sulfate. In some embodiments, the conjugate is biodegradable. In some embodiments, the glycosaminoglycan is between 5 kD and 20 kD.

In certain embodiments, this invention is directed to low molecular weight lipid-polymer conjugate comprising a GAG wherein the average molecular weight of said GAG is between 5 kd to 90 kd. In some embodiments, the average molecular weight of said GAG is between 5 kD to 60 kD. In some embodiments, the average molecular weight of said GAG is between 5 kD to 40 kD. In some embodiments, the average molecular weight of said GAG is between 5 kD to 15 kD. In some embodiments, the average molecular weight of said GAG is between 5 kD to 20 kD. In some embodiments, the average molecular weight of said GAG is between 5 kD to 25 kD.

Cell surface GAG play a key role in protecting cells from diverse damaging agents and processes, such as reactive oxygen species and free radicals, endotoxins, cytokines, invasion promoting enzymes, and agents that induce and/or facilitate degradation of extracellular matrix and basal membrane, cell invasiveness, white cell extravasation and infiltration, chemotaxis, and others. In addition, cell surface GAG protect cells from bacterial, viral and parasite infection, and their stripping exposes the cell to interaction and subsequent internalization of the microorganism. Enrichment of cell surface GAG would thus assist in protection of the cell from injurious processes. Thus, in some embodiments of the invention, PLA2 inhibitors were conjugated to GAGs or GAG-mimicking molecules. In other embodiments, these Lipid-conjugates, provides wide-range protection from diverse injurious processes, and are effective in amelioration of diseases that requires cell protection from injurious biochemical mediators.

In certain embodiments, GAG-mimicking molecule may be, inter alia, a negatively charged molecule. In some embodiments, GAG-mimicking molecule may be, inter alia, a salicilate derivative. In some embodiments, GAG-mimicking molecule may be, inter alia, a dicarboxylic acid.

### Preparation of Compounds

The preparation of some high molecular weight Lipid-conjugates is the subject of U.S. Pat. No. 5,064,817, which is incorporated herein by reference. These synthetic methods are reiterated below and are considered to be applicable as well to the preparation of low molecular, i.e. Lipid-conjugates comprising monomers and dimers as the conjugated moiety, with modifications in the procedure as readily evident to one skilled in the art.

When the starting compound chosen for the conjugated moiety has a substituent which is or can be rendered reactive to a substituent on the starting Lipid compound, the conjugated carrier moiety may be linked directly to lipid molecule(s) to produce the a Lipid-conjugate. When it does to not, a bifunctional linking starting material can be used to link the two molecules indirectly.

Lipid-conjugates are prepared by linking a polar conjugate, e.g., a monomer or polymer, directly or indirectly to a PL moiety according to the general reaction schemes delineated in U.S. Pat. No. 5,064,817 and according to US Publication 2011-0130555.

For example, with acylated PE used as precursor for the PE conjugate, various lengths of dicarboxylic acids can be used as spacers. These acids can be linked to natural, semi-synthetic or synthetic PE.

For example, PE can be linked to aminodextran indirectly as delineated in U.S. Pat. No. 5,064,817 and US Publication 2011-0130555.

Polymers with carboxylic groups, such as polyamino acids, carboxymethyl cellulose or polymers to which fatty acids have been linked, can be linked directly to PE according to the scheme delineated in U.S. Pat. No. 5,064,817.

It is to be understood that these examples are given by way of illustration only and are not to be construed as limiting the invention either in spirit of in scope, as many modifications both in reagents and methods could be possible to those skilled in the art. Based on the wide spectrum of pharmacological properties exhibited by Lipid-conjugates, it is likely that compounds covered by Formula I-XXI, in addition to those explicitly described above, have the same valuable biological activities demonstrate to be useful in the methods of treating disease described below.

In certain embodiments, the invention provides processes for the preparation of a compound represented by the structure of the general formula (A):

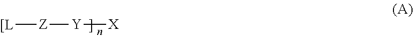

wherein
L is a lipid or a phospholipid;
Z is either nothing, ethanolamine, serine, inositol, choline, or glycerol;
Y is either nothing or a spacer group ranging in length from 2 to 30 atoms;

X is a physiologically acceptable monomer, dimer, oligomer, or polymer, wherein X is a glycosaminoglycan; and
n is a number from 1 to 1000;
wherein any bond between L, Z, Y and X is either an amide or an esteric bond,
including, inter alia, the steps of:
conjugating L to Z;
conjugating Z to Y;
conjugating Y to X;
wherein if Z is nothing, L is conjugated directly to Y,
if Y is nothing, Z is conjugated directly to X, and
if Y and Z are nothing, L is conjugated directly to X,
thereby preparing a compound represented by the structure of the general formula (A).

In some embodiments, the invention provides processes for the preparation of a compound represented by the structure of the general formula (I):

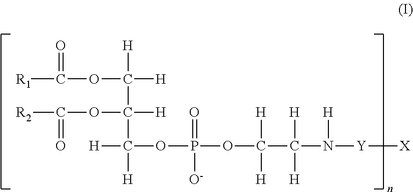

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is either a physiologically acceptable monomer, dimer, oligomer or a physiologically acceptable polymer, wherein X is a glycosaminoglycan; and
n is a number from 1 to 1,000;
wherein if Y is nothing the phosphatidylethanolamine is directly linked to X via an amide bond and if Y is a spacer, the spacer is directly linked to X via an amide or an esteric bond and to the phosphatidylethanolamine via an amide bond, including, inter alia, the steps of: conjugating the phosphatidylethanolamine to Y; and
conjugating Y to X;
if Y is nothing, the phosphatidylethanolamine is conjugated directly to X,
thereby preparing a compound represented by the structure of the general formula (I).

In some embodiments of the invention, the phosphatidylethanolamine is the chemical moiety represented by the structure of:

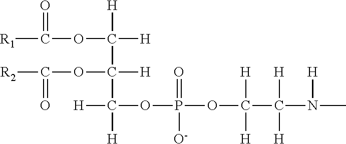

wherein R₁ and R₂ are defined herein.

In some embodiments, the invention provides processes for the preparation of a compound represented by the structure of the general formula (II):

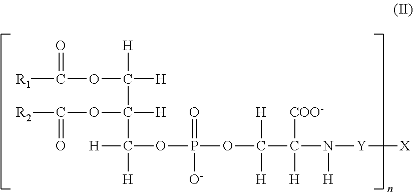

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a physiologically acceptable monomer, dimer, oligomer or polymer wherein x is a glycosaminoglycan; and
n is a number from 1 to 1000;
wherein if Y is nothing the phosphatidylserine is directly linked to X via an amide bond and if Y is a spacer, the spacer is directly linked to X via an amide or an esteric bond and to the phosphatidylserine via an amide bond, including, inter alia, the steps of:
conjugating the phosphatidylserine to Y;
conjugating Y to X;
if Y is nothing, the phosphatidylserine is conjugated directly to X,
thereby preparing a compound represented by the structure of the general formula (II).

In certain embodiments of the invention, the phosphatidylserine is the chemical moiety represented by the structure of:

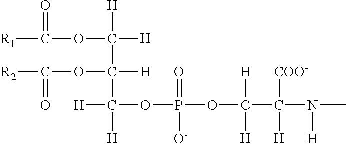

wherein R₁ and R₂ are defined herein.

In some embodiments, the invention provides processes for the preparation of a compound represented by the structure of the general formula (III):

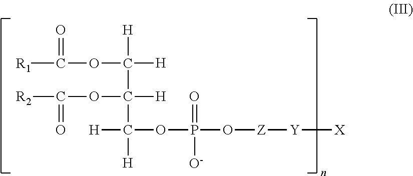

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phosphatidyl, Z, Y and X is either an amide or anesteric bond, including, inter alia, the steps of:
conjugating the phosphatidyl to Z;
conjugating Z to Y;
conjugating Y to X;
wherein if Z is nothing, the phosphatidyl is conjugated directly to Y,
if Y is nothing, Z is conjugated directly to X, and
if Y and Z are nothing, the phosphatidyl is conjugated directly to X,
thereby preparing a compound represented by the structure of the general formula (III).

In some embodiments of the invention, the phosphatidyl may be the chemical moiety represented by the structure of:

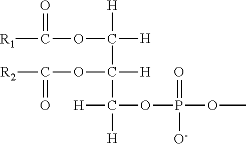

wherein R₁ and R₂ are defined herein.

In some embodiments, the invention provides processes for the preparation of a compound represented by the structure of the general formula (IV):

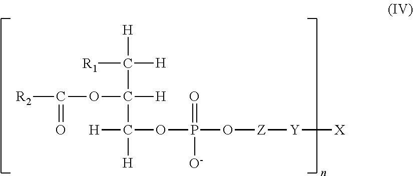

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond, including, inter alia, the steps of:
conjugating the phospholipid to Z;
conjugating Z to Y;
conjugating Y to X;
wherein if Z is nothing, the phospholipid is conjugated directly to Y,
if Y is nothing, Z is conjugated directly to X, and
if Y and Z are nothing, the phospholipid is conjugated directly to X,
thereby preparing a compound represented by the structure of the general formula (IV).

In some embodiments of the invention, the phospholipid may be the chemical moiety represented by the structure of:

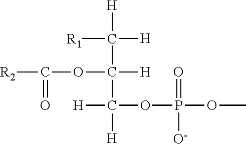

wherein R₁ and R₂ are defined herein.

In some embodiments, the invention provides processes for the preparation of a compound represented by the structure of the general formula (V):

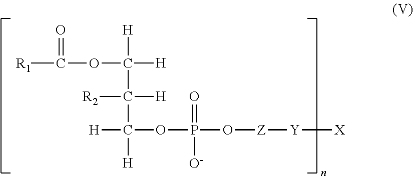

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond, including, inter alia, the steps of:
conjugating the phospholipid to Z;
conjugating Z to Y;
conjugating Y to X;
wherein if Z is nothing, the phospholipid is conjugated directly to Y,
if Y is nothing, Z is conjugated directly to X, and
if Y and Z are nothing, the phospholipid is conjugated directly to X,
thereby preparing a compound represented by the structure of the general formula (V).

In some embodiments of the invention, the phospholipid may be the chemical moiety represented by the structure of:

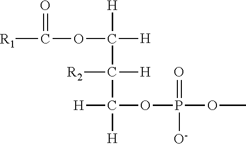

wherein R₁ and R₂ are defined herein.

In some embodiments, the invention provides processes for the preparation of a compound represented by the structure of the general formula (VI):

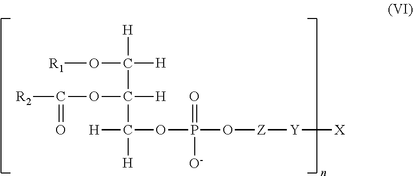

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond, including, inter alia, the steps of:
conjugating the phospholipid to Z;
conjugating Z to Y;
conjugating Y to X;
wherein if Z is nothing, the phospholipid is conjugated directly to Y,
if Y is nothing, Z is conjugated directly to X, and
if Y and Z are nothing, the phospholipid is conjugated directly to X, thereby preparing a compound represented by the structure of the general formula (VI).

In some embodiments of the invention, the phospholipid may be the chemical moiety represented by the structure of:

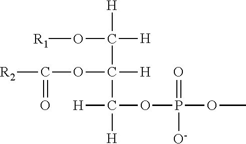

wherein R₁ and R₂ are defined herein.

In some embodiments, the invention provides processes for the preparation of a compound represented by the structure of the general formula (VII):

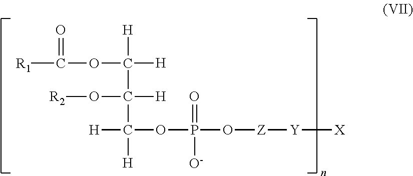

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms:
Z is either nothing, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond, including, inter alia, the steps of:
conjugating the phospholipid to Z;
conjugating Z to Y;
conjugating Y to X;
wherein if Z is nothing, the phospholipid is conjugated directly to Y,
if Y is nothing, Z is conjugated directly to X, and
if Y and Z are nothing, the phospholipid is conjugated directly to X, thereby preparing a compound represented by the structure of the general formula (VII).

In some embodiments of the invention, the phospholipid may be the chemical moiety represented by the structure of:

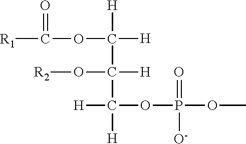

wherein R₁ and R₂ are defined herein.

In some embodiments, the invention provides processes for the preparation of a compound represented by the structure of the general formula (VIII):

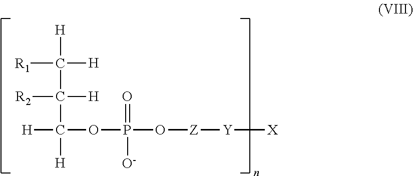

wherein
R₁ is a linear, saturated, mono-unsaturated, or poly-unsaturated, alkyl chain ranging in length from 2 to 30 carbon atoms;

R₂ is either hydrogen or a linear, saturated, mono-unsaturated, or poly-unsaturated, alkyl chain ranging in length from 2 to 30 carbon atoms:
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond, including, inter alia, the steps of:
conjugating the phospholipid to Z;
conjugating Z to Y;
conjugating Y to X;
wherein if Z is nothing, the phospholipid is conjugated directly to Y,
if Y is nothing, Z is conjugated directly to X, and
if Y and Z are nothing, the phospholipid is conjugated directly to X, thereby preparing a compound represented by the structure of the general formula (VIII).

In some embodiments of the invention, the phospholipid may be the chemical moiety represented by the structure of:

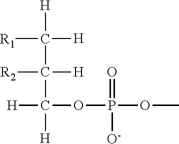

wherein R₁ and R₂ are defined herein.

In some embodiments, the invention provides processes for the preparation of a compound represented by the structure of the general formula (IX):

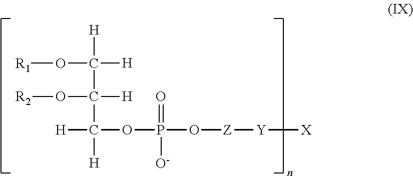

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond, including, inter alia, the steps of:
conjugating the phospholipid to Z;
conjugating Z to Y;
conjugating Y to X;
wherein if Z is nothing, the phospholipid is conjugated directly to Y,
if Y is nothing, Z is conjugated directly to X, and
if Y and Z are nothing, the phospholipid is conjugated directly to X, thereby preparing a compound represented by the structure of the general formula (IX).

In some embodiments of the invention, the phospholipid may be the chemical moiety represented by the structure of:

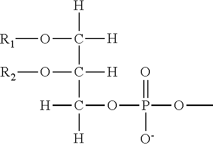

wherein R₁ and R₂ are defined herein.

In some embodiments, the invention provides processes for the preparation of a compound represented by the structure of the general formula (IXa):

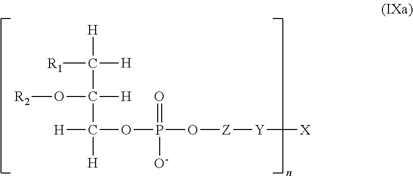

R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond, including, inter alia, the steps of:
conjugating the phospholipid to Z;
conjugating Z to Y;
conjugating Y to X;
wherein if Z is nothing, the phospholipid is conjugated directly to Y,
if Y is nothing, Z is conjugated directly to X, and
if Y and Z are nothing, the phospholipid is conjugated directly to X, thereby preparing a compound represented by the structure of the general formula (IXa).

In some embodiments of the invention, the phospholipid may be the chemical moiety represented by the structure of:

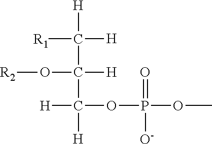

wherein R₁ and R₂ are defined herein.

In some embodiments, the invention provides processes for the preparation of a compound represented by the structure of the general formula (IXb):

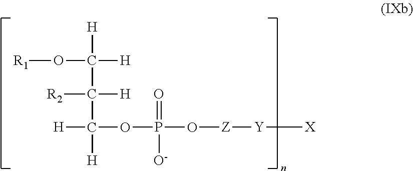

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, ethanolamine, serine, inositol, choline, or glycerol;
Y is either nothing or a spacer group ranging in length from 2 to 30 atoms:
X is a physiologically acceptable monomer, dimer, oligomer, or polymer, wherein x is a glycosaminoglycan; and
n is a number from 1 to 1000;
wherein any bond between the phospholipid, Z, Y and X is either an amide or an esteric bond, including, inter alia, the steps of:
conjugating the phospholipid to Z;
conjugating Z to Y;
conjugating Y to X;
wherein if Z is nothing, the phospholipid is conjugated directly to Y,
if Y is nothing, Z is conjugated directly to X, and
if Y and Z are nothing, the phospholipid is conjugated directly to X, thereby preparing a compound represented by the structure of the general formula (IXb).

In some embodiments of the invention, the phospholipid may be the chemical moiety represented by the structure of:

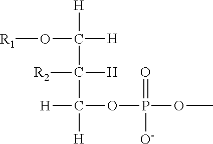

wherein R₁ and R₂ are defined herein.

In some embodiments, the invention provides processes for the preparation of a compound represented by the structure of the general formula (X):

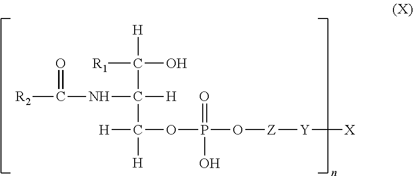

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing ethanolamine, serine, inositol, choline, or glycerol;
Y is either nothing or a spacer group ranging in length from 2 to 30 atoms;
X is a physiologically acceptable monomer, dimer, oligomer, or polymer, wherein x is a glycosaminoglycan; and
n is a number from 1 to 1000;
wherein any bond between the ceramide phosphoryl, Z, Y and X is either an amide or an esteric bond, including, inter alia, the steps of:
conjugating the ceramide phosphoryl to Z;
conjugating Z to Y;
conjugating Y to X;
wherein if Z is nothing, the ceramide phosphoryl is conjugated directly to Y,
if Y is nothing, Z is conjugated directly to X, and
if Y and Z are nothing, the ceramide phosphoryl is conjugated directly to X, thereby preparing a compound represented by the structure of the general formula (X).

In some embodiments of the invention, the ceramide phosphoryl may be the chemical moiety represented by the structure of:

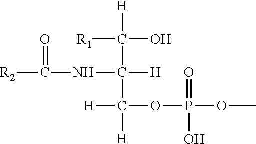

wherein R₁ and R₂ are defined herein.

In some embodiments, the invention provides processes for the preparation of a compound represented by the structure of the general formula (XI):

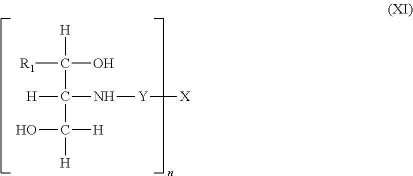

wherein
R₁ is a linear, saturated, mono-unsaturated, or poly-unsaturated, alkyl chain ranging in length from 2 to 30 carbon atoms;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein if Y is nothing the sphingosyl is directly linked to X via an amide bond and if Y is a spacer, the spacer is directly linked to X and to the sphingosyl via an amide bond and to X via an amide or an esteric bond, including, inter alia, the steps of:
conjugating the sphingosyl to Y;
conjugating Y to X;
wherein if Y is nothing, the sphingosyl is conjugated directly to X, thereby preparing a compound represented by the structure of the general formula (XI).

In some embodiments of the invention, the sphingosyl may be the chemical moiety

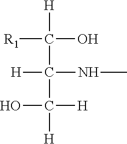

wherein R₁ is defined herein.

In some embodiments, the invention provides processes for the preparation of a compound represented by the structure of the general formula (XII):

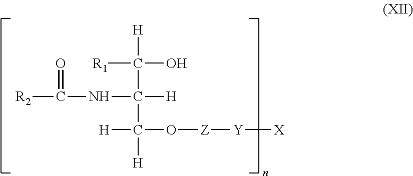

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
L is ceramide;
Z is either nothing, ethanolamine, serine, inositol, choline, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the ceramide, Z, Y and X is either an amide or an esteric bond, including, inter alia, the steps of:
conjugating the ceramide to Z;
conjugating Z to Y;
conjugating Y to X;
wherein if Z is nothing, the ceramide is conjugated directly to Y,
if Y is nothing, Z is conjugated directly to X, and
if Y and Z are nothing, the ceramide is conjugated directly to X, thereby preparing a compound represented by the structure of the general formula (XII).

In some embodiments of the invention, the ceramide may be the chemical moiety represented by the structure of:

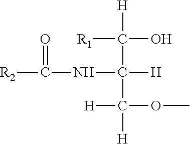

wherein R₁ and R₂ are defined herein.

In some embodiments, the invention provides processes for the preparation of a compound represented by the structure of the general formula (XIII):

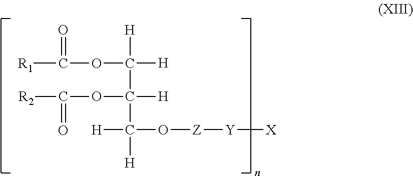

wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the diglyceryl, Z, Y and X is either an amide or an esteric bond, including, inter alia, the steps of:
conjugating the diglyceryl to Z:
conjugating Z to Y;
conjugating Y to X;
wherein if Z is nothing, the diglyceryl is conjugated directly to Y,
if Y is nothing, Z is conjugated directly to X, and
if Y and Z are nothing, the diglyceryl is conjugated directly to X, thereby preparing a compound represented by the structure of the general formula (XIII).

In some embodiments of the invention, the diglyceryl may be the chemical moiety represented by the structure of:

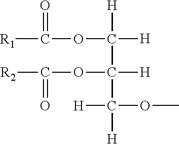

wherein R₁ and R₂ are defined herein.

In some embodiments, the invention provides processes for the preparation of a compound represented by the structure of the general formula (XIV):

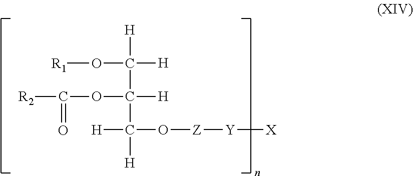

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the glycerolipid, Z, Y and X is either an amide or an esteric bond, including, inter alia, the steps of:
conjugating the glycerolipid to Z;
conjugating Z to Y;
conjugating Y to X;
wherein if Z is nothing, the glycerolipid is conjugated directly to Y,
if Y is nothing, Z is conjugated directly to X, and
if Y and Z are nothing, the glycerolipid is conjugated directly to X, thereby preparing a compound represented by the structure of the general formula (XIV).

In some embodiments of the invention, the glycerolipid may be the chemical moiety represented by the structure of:

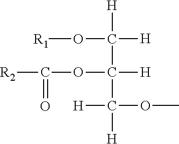

wherein R₁ and R₂ are defined herein.

In some embodiments, the invention provides processes for the preparation of a compound represented by the structure of the general formula (XV):

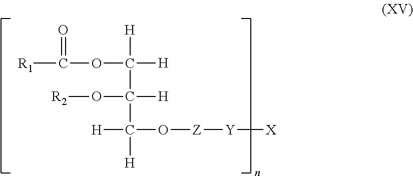

wherein
R₁ is a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the glycerolipid, Z, Y and X is either an amide or an esteric bond, including, inter alia, the steps of:
conjugating the glycerolipid to Z;
conjugating Z to Y;
conjugating Y to X;
wherein if Z is nothing, the glycerolipid is conjugated directly to Y,
if Y is nothing, Z is conjugated directly to X, and
if Y and Z are nothing, the glycerolipid is conjugated directly to X, thereby preparing a compound represented by the structure of the general formula (XV).

In some embodiments of the invention, the glycerolipid may be the chemical moiety represented by the structure of:

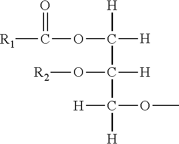

wherein R₁ and R₂ are defined herein.

In some embodiments, the invention provides processes for the preparation of a compound represented by the structure of the general formula (XVI):

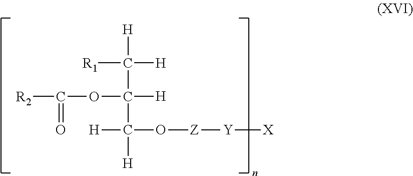

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond, including, inter alia, the steps of:
conjugating the lipid to Z;
conjugating Z to Y;
conjugating Y to X;
wherein if Z is nothing, the lipid is conjugated directly to Y,
if Y is nothing, Z is conjugated directly to X, and
if Y and Z are nothing, the lipid is conjugated directly to X, thereby preparing a compound represented by the structure of the general formula (XVI).

In some embodiments of the invention, the lipid may be the chemical moiety represented by the structure of:

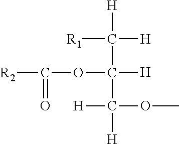

wherein R₁ and R₂ are defined herein.

In some embodiments, the invention provides processes for the preparation of a compound represented by the structure of the general formula (XVII):

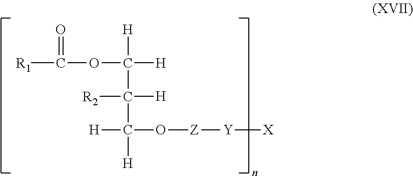

wherein
R₁ is either hydrogen or a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond, including, inter alia, the steps of:
conjugating the lipid to Z;
conjugating Z to Y;
conjugating Y to X;
wherein if Z is nothing, the lipid is conjugated directly to Y,
if Y is nothing, Z is conjugated directly to X, and
if Y and Z are nothing, the lipid is conjugated directly to X, thereby preparing a compound represented by the structure of the general formula (XVII).

In some embodiments of the invention, the lipid may be the chemical moiety represented by the structure of:

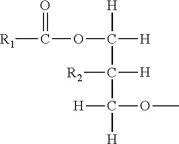

wherein R₁ and R₂ are defined herein.

In some embodiments, the invention provides processes for the preparation of a compound represented by the structure of the general formula (XVIll):

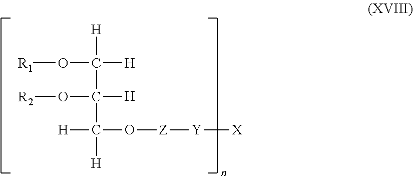

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms:
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond, including, inter alia, the steps of:
conjugating the lipid to Z;
conjugating Z to Y;
conjugating Y to X;
wherein if Z is nothing, the lipid is conjugated directly to Y,
if Y is nothing, Z is conjugated directly to X, and
if Y and Z are nothing, the lipid is conjugated directly to X, thereby preparing a compound represented by the structure of the general formula (XVIII).

In some embodiments of the invention, the lipid may be the chemical moiety represented by the structure of:

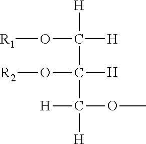

wherein R₁ and R₂ are defined herein.

In some embodiments, the invention provides processes for the preparation of a compound represented by the structure of the general formula (XIX):

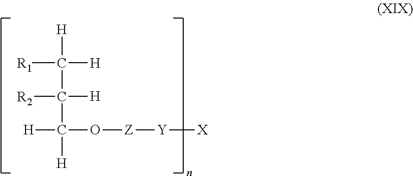

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond, including, inter alia, the steps of:
conjugating the lipid to Z;
conjugating Z to Y;
conjugating Y to X;
wherein if Z is nothing, the lipid is conjugated directly to Y,
if Y is nothing, Z is conjugated directly to X, and
if Y and Z are nothing, the lipid is conjugated directly to X, thereby preparing a compound represented by the structure of the general formula (XIX).

In some embodiments of the invention, the lipid may be the chemical moiety represented by the structure of:

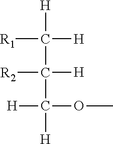

wherein R₁ and R₂ are defined herein.

In some embodiments, the invention provides processes for the preparation of a compound represented by the structure of the general formula (XX):

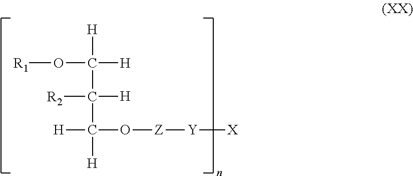

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond, including, inter alia, the steps of:
conjugating the lipid to Z;
conjugating Z to Y;
conjugating Y to X;
wherein if Z is nothing, the lipid is conjugated directly to Y,
if Y is nothing, Z is conjugated directly to X, and
if Y and Z are nothing, the lipid is conjugated directly to X, thereby preparing a compound represented by the structure of the general formula (XX).

In some embodiments of the invention, the lipid may be the chemical moiety represented by the structure of:

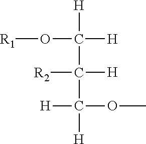

wherein R₁ and R₂ are defined herein.

In some embodiments, the invention provides processes for the preparation of a compound represented by the structure of the general formula (XXI):

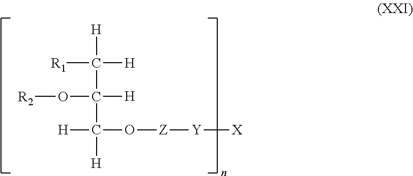

wherein
R₁ is either hydrogen or a linear, saturated, mono-unsaturated, or poly-unsaturated, alkyl chain ranging in length from 2 to 30 carbon atoms;
R₂ is either hydrogen or a linear, saturated, mono-unsaturated, or poly-unsaturated, alkyl chain ranging in length from 2 to 30 carbon atoms;
Z is either nothing, choline, phosphate, inositol, or glycerol;
Y is either nothing or a spacer group ranging in length from 2 to 30 atoms;
X is a physiologically acceptable monomer, dimer, oligomer or polymer, wherein x is a glycosaminoglycan; and
n is a number from 1 to 1000;
wherein any bond between the lipid, Z, Y and X is either an amide or an esteric bond, including, inter alia, the steps of:
conjugating the lipid to Z;
conjugating Z to Y;
conjugating Y to X;
wherein if Z is nothing, the lipid is conjugated directly to Y,
if Y is nothing, Z is conjugated directly to X, and
if Y and Z are nothing, the lipid is conjugated directly to X, thereby preparing a compound represented by the structure of the general formula (XXI).

In some embodiments of the invention, the lipid may be the chemical moiety represented by the structure of:

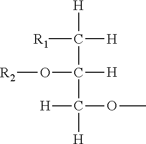

wherein R₁ and R₂ are defined herein.

In certain embodiments, the conjugating according to the invention may be performed by eliminating a water molecule, thereby forming amide or esteric bonds. In some embodiments, the conjugating may be performed in the presence of a detergent. In some embodiments, the conjugating may be induced by ultrasonic radiation.

In certain embodiments, any compound according to the invention may be prepared by a conjugation process performed by eliminating a water molecule, thereby forming amide or esteric bonds. In some embodiments, any compound according to the invention may be prepared by a conjugation process in the presence of a detergent. In some embodiments, any compound according to the invention may be prepared by a conjugation process induced by ultrasonic radiation.

In certain embodiments of the invention, the conjugation of the phosphatidylethanolamine and chondroitin sulfate is performed in the presence of a detergent. In some of these embodiments the detergent may be, inter alia, DDAB. Of course any other appropriate detergent may be used.

In some embodiments of the invention, the conjugation of the phosphatidylethanolamine and hyaluronic acid is induced by sonication.

### Methods of Treating Disease Based on PL Conjugates

In certain embodiments of the invention, the Lipid-conjugates described herein can be used to treat disease, through exerting at least one of their many pharmacological activities, among which are amelioration, or prevention, of tissue injury arising in the course of pathological disease states by stabilizing cell membranes; limiting oxidative damage to cell and blood components; limiting cell proliferation, cell extravasation and (tumor) cell migratory behavior; suppressing immune responses; or attenuating physiological reactions to stress, as expressed in elevated chemokine levels. The medicinal properties of these compounds are readily exemplified in using animal models of the particular disease in which it is desired to use the drug. The patients to whom the lipid or PL conjugates should be administered are those that are experiencing symptoms of disease or who are at risk of contracting the disease or experiencing a recurrent episode or exacerbation of the disease. The efficacy of these compounds in cellular and animal models of disease are described below in The Examples.

The methods of treatment described herein can be used to treat any suitable subject. The term “subject,” as used herein, refers to any animal, including but not limited to, any suitable mammal, including primates, such as monkeys and humans, horses, cows, cats, dogs, rabbits, and rodents, such as rats and mice. In certain embodiments, the subject to be treated is human.

The combination of lipids, such as, but not limited to phosphatidylethanolamine and phosphatidylserine, with additional monomer or polymer moieties, is thus a practical route to the production of new drugs for medical purposes, provided that the resultant chemical composition displays the desired range of pharmacological properties. In the cases described herein, the diversity of biological activities and the effectiveness in disease exhibited by the compounds far exceed the properties anticipated by use of the starting materials themselves, when administered alone or in combination. However, it is likely that the PL conjugate compounds, alone or in combination, will prove to be valuable drugs when adapted to methods of disease treatment other than to those conditions specifically described herein.

In certain embodiments, the invention provides methods of treating a subject afflicted with a disease related to bronchitis.

In certain embodiments, the invention provides methods of treating a subject suffering from bronchitis, including, inter alia, the step of administering to a subject an effective amount of a lipid or phospholipid moiety bonded to a physiologically acceptable monomer, dimer, oligomer, or polymer.

In certain embodiments, the invention provides methods of preventing bronchitis in a subject, including, inter alia, the step of administering to a subject an effective amount of a lipid or phospholipid moiety bonded to a physiologically acceptable monomer, dimer, oligomer, or polymer.

In certain embodiments, the invention provides a use of a lipid or phospholipid moiety bonded to a physiologically acceptable monomer, dimer, oligomer, or polymer, in the preparation of a pharmaceutical composition for treating a subject suffering from bronchitis.

In certain embodiments, the invention provides a use of a lipid or phospholipid moiety bonded to a physiologically acceptable monomer, dimer, oligomer, or polymer, in the preparation of a pharmaceutical composition for preventing bronchitis in a subject.

In some embodiments of the invention, the treatment requires controlling the expression, to production, and activity of phospholipase enzymes. In some embodiments, the treatment requires controlling the production and/or action of lipid mediators. In some embodiments, the treatment requires amelioration of damage to glycosaminoglycans (GAG) and proteoglycans. In some embodiments, the treatment requires controlling the production and action of oxidants, oxygen radicals and nitric oxide. In some embodiments, the treatment requires anti-oxidant therapy. In some embodiments, the treatment requires anti-endotoxin therapy. In some embodiments, the treatment requires controlling the expression, production or action of cytokines, chemokines, adhesion molecules or interleukines. In some embodiments, the treatment requires protection of lipoproteins from damaging agents. In some embodiments, the treatment requires controlling the proliferation of cells. In some embodiments, the treatment requires controlling of angiogenesis and organ vascularization. In some embodiments, the treatment requires inhibition of invasion-promoting enzymes. In some embodiments, the treatment requires controlling of cell invasion. In some embodiments, the invading cells are white blood cells. In some embodiments, the invading cells are cancer cells. In some embodiments, the treatment requires controlling of white cell activation, adhesion or extravasation. In some embodiments, the treatment requires amelioration of ischemia or reperfusion injury. In some embodiments, the treatment requires inhibition of lymphocyte activation. In some embodiments, the treatment requires protection of blood brain barrier. In some embodiments, the treatment requires control of neurotransmitter production and action. In some embodiments, the treatment requires controlling of blood vessel and airway contraction. In some embodiments, the treatment requires extracorporeal tissue preservation.

In certain embodiments of the invention, the lipid mediator is a glycerolipid. In some embodiments, the lipid mediator is a phospholipid. In some embodiments, the lipid mediator is sphingolipid. In some embodiments, the lipid mediator is a sphingosine. In some embodiments, the lipid mediator is ceramide. In some embodiments, the lipid mediator is a fatty acid. In some embodiments, the fatty acid is arachidonic acid. In some embodiments, the lipid mediator is an arachidonic acid-derived eicosanoid. In some embodiments, the lipid mediator is a platelet activating factor (PAF). In some embodiments, the lipid mediator is a lysophospholipid.

In certain embodiments of the invention, the damaging agent is a phospholipase. In some embodiments, the damaging agent is a reactive oxygen species (ROS). In some embodiments, the damaging agent is a free radical. In some embodiments, the damaging agent is a lysophospholipid. In some embodiments, the damaging agent is a fatty acid or a derivative thereof. In some embodiments, the damaging agent is hydrogen peroxide. In some embodiments, the damaging agent is a phospholipid. In some embodiments, the damaging agent is an oxidant. In some embodiments, the damaging agent is a cationic protein. In some embodiments, the damaging agent is a streptolysin. In some embodiments, the damaging agent is a protease. In some embodiments, the damaging agent is a hemolysin. In some embodiments, the damaging agent is a sialidase.

In certain embodiments of the invention, the invasion-promoting enzyme is collagenase. In some embodiments, the invasion-promoting enzyme is matrix-metaloproteinase (MMP). In some embodiments, the invasion-promoting enzyme is heparinase. In some embodiments, the invasion-promoting enzyme is heparanase. In some embodiments, the invasion-promoting enzyme is hyaluronidase. In some embodiments, the invasion-promoting enzyme is gelatinase. In some embodiments, the invasion-promoting enzyme is chondroitinase. In some embodiments, the invasion-promoting enzyme is dermatanase. In some embodiments, the invasion-promoting enzyme is keratanase. In some embodiments, the invasion-promoting enzyme is protease. In some embodiments, the invasion-promoting enzyme is lyase. In some embodiments, the invasion-promoting enzyme is hydrolase. In some embodiments, the invasion-promoting enzyme is a glycosaminoglycan degrading enzyme. In some embodiments, the invasion-promoting enzyme is a proteoglycan degrading enzyme.

In certain embodiments of the invention, the physiologically acceptable monomer is salicylate. In some embodiments, the physiologically acceptable monomer is salicylic acid. In some embodiments, the physiologically acceptable monomer is aspirin. In some embodiments, the physiologically acceptable monomer is a monosaccharide. In some embodiments, the physiologically acceptable monomer is lactobionic acid. In some embodiments, the physiologically acceptable monomer is glucoronic acid. In some embodiments, the physiologically acceptable monomer is maltose. In some embodiments, the physiologically acceptable monomer is an amino acid. In some embodiments, the physiologically acceptable monomer is glycine. In some embodiments, the physiologically acceptable monomer is a carboxylic acid. In some embodiments, the physiologically acceptable monomer is an acetic acid. In some embodiments, the physiologically acceptable monomer is a butyric acid. In some embodiments, the physiologically acceptable monomer is a dicarboxylic acid. In some embodiments, the physiologically acceptable monomer is a glutaric acid. In some embodiments, the physiologically acceptable monomer is succinic acid. In some embodiments, the physiologically acceptable monomer is a fatty acid. In some embodiments, the physiologically acceptable monomer is dodecanoic acid. In some embodiments, the physiologically acceptable monomer is didodecanoic acid. In some embodiments, the physiologically acceptable monomer is bile acid. In some embodiments, the physiologically acceptable monomer is cholic acid. In some embodiments, the physiologically acceptable monomer is cholesterylhemmisuccinate.

In certain embodiments of the invention, the physiologically acceptable dimer or oligomer is physiologically acceptable dimer or oligomer is a dipeptide. In some embodiments, the physiologically acceptable dimer or oligomer is a disaccharide. In some embodiments, the physiologically acceptable dimer or oligomer is a trisaccharide. In some embodiments, the physiologically acceptable dimer or oligomer is an oligosaccharide. In some embodiments, the physiologically acceptable dimer or oligomer is an oligopeptide. In some embodiments, the physiologically acceptable dimer or oligomer is a di- or trisaccharide monomer unit of glycosaminoglcans. In some embodiments, the physiologically acceptable dimer or oligomer is hyaluronic acid. In some embodiments, the physiologically acceptable dimer or oligomer is heparin. In some embodiments, the physiologically acceptable dimer or oligomer is heparan sulfate. In some embodiments, the physiologically acceptable dimer or oligomer is keratin. In some embodiments, the physiologically acceptable dimer or oligomer is keratan sulfate. In some embodiments, the physiologically acceptable dimer or oligomer is chondroitin. In some embodiments, the chondroitin is chondroitin sulfate. In some embodiments, the chondroitin is chondroitin-4-sulfate. In some embodiments, the chondroitin is chondroitin-6-sulfate. In some embodiments, the physiologically acceptable dimer or oligomer is dermatin. In some embodiments, the physiologically acceptable dimer or oligomer is dermatan sulfate. In some embodiments, the physiologically acceptable dimer or oligomer is dextran. In some embodiments, the physiologically acceptable dimer or oligomer is polygeline (‘Haemaccel’). In some embodiments, the physiologically acceptable dimer or oligomer is alginate, In some embodiments, the physiologically acceptable dimer or oligomer is hydroxyethyl starch (Hetastarch). In some embodiments, the physiologically acceptable dimer or oligomer is ethylene glycol. In some embodiments, the physiologically acceptable dimer or oligomer is carboxylated ethylene glycol.

In certain embodiments of the invention, the physiologically acceptable polymer is a glycosaminoglycan. In some embodiments, the physiologically acceptable polymer is hyaluronic acid. In some embodiments, the physiologically acceptable polymer is heparin. In some embodiments, the physiologically acceptable polymer is heparan sulfate. In some embodiments, the physiologically acceptable polymer is chondroitin. In some embodiments, the chondroitin is chondroitin-4-sulfate. In some embodiments, the chondroitin is chondroitin-6-sulfate. In some embodiments, the physiologically acceptable polymer is keratin. In some embodiments, the physiologically acceptable polymer is keratan sulfate. In some embodiments, the physiologically acceptable polymer is dermatin. In some embodiments, the physiologically acceptable polymer is dermatan sulfate. In some embodiments, the physiologically acceptable polymer is carboxymethylcellulose. In some embodiments, the physiologically acceptable polymer is dextran. In some embodiments, the physiologically acceptable polymer is polygeline (‘Haemaccel’). In some embodiments, the physiologically acceptable polymer is alginate. In some embodiments, the physiologically acceptable polymer is hydroxyethyl starch (‘Hetastarch’). In some embodiments, the physiologically acceptable polymer is polyethylene glycol. In some embodiments, the physiologically acceptable polymer is polycarboxylated polyethylene glycol.

In certain embodiments of the invention, the lipid or phospholipid moiety is phosphatidic acid. In some embodiments, lipid or phospholipid moiety is an acyl glycerol. In some embodiments, lipid or phospholipid moiety is monoacylglycerol. In some embodiments, lipid or phospholipid moiety is diacylglycerol. In some embodiments, lipid or phospholipid moiety is triacylglycerol. In some embodiments, lipid or phospholipid moiety is sphingosine. In some embodiments, lipid or phospholipid moiety is sphingomyelin. In some embodiments, lipid or phospholipid moiety is ceramide. In some embodiments, lipid or phospholipid moiety is phosphatidylethanolamine. In some embodiments, lipid or phospholipid moiety is phosphatidylserine. In some embodiments, lipid or phospholipid moiety is phosphatidylcholine. In some embodiments, lipid or phospholipid moiety is phosphatidylinositol. In some embodiments, lipid or phospholipid moiety is phosphatidylglycerol. In some embodiments, lipid or phospholipid moiety is an ether or alkyl phospholipid derivative thereof.

In some embodiments, the invention provides methods of treating a subject afflicted with a disease, wherein the treatment of the disease requires controlling phospholipase A2 activities; controlling the production and/or action of lipid mediators, such as eicosanoids, platelet activating factor (PAF) and lyso-phospholipids; amelioration of damage to cell surface glycosaminoglycans (GAG) and proteoglycans; controlling the production of oxygen radicals and nitric oxide; protection of cells, tissues, and plasma lipoproteins from damaging agents, such as reactive oxygen species (ROS) and phospholipases; anti-oxidant therapy; anti-endotoxin therapy; controlling of cytokine, chemokine and interleukine production; controlling the proliferation of cells, including smooth muscle cells, endothelial cells and skin fibroblasts; controlling of angiogenesis and organ vascularization; inhibition of invasion-promoting enzymes, such as collagenase, heparinase, heparanase and hyaluronidase; controlling of cell invasion; controlling of white cell activation, adhesion and extravasation; amelioration of ischemiaireperfusion injury, inhibition of lymphocyte activation; controlling of blood vessel and airway contraction; protection of blood brain barrier; controlling of neurotransmitter (e.g., dopamine) production and action (e.g., acethylcholine); extracorporeal tissue preservation or any combination thereof.

In certain embodiments of the invention, the term “controlling” refers to inhibiting the production and action of the above mentioned factors in order to maintain their activity at the normal basal level and suppress their activation in pathological conditions.

In certain embodiments of the invention, the physiologically acceptable monomer is either a salicylate, salicylic acid, aspirin, a monosaccharide, lactobionic acid, maltose, an amino acid, glycine, carboxylic acid, acetic acid, butyric acid, dicarboxylic acid, glutaric acid, succinic acid, fatty acid, dodecanoic acid, didodecanoic acid, bile acid, cholic acid, cholesterylhemmisuccinate; or wherein the physiologically acceptable dimer or oligomer is a dipeptide, a disaccharide, a trisaccharide, an oligopeptide, or a di- or trisaccharide monomer unit of heparin, heparan sulfate, keratin, keratan sulfate, chondroitin, chondroitin-6-sulfate, chondroitin-4-sulfate, dermatin, dermatan sulfate, dextran, or hyaluronic acid; or wherein the physiologically acceptable polymer is a glycosaminoglycan, polygelin (‘haemaccel’), alginate, hydroxyethyl starch (hetastarch), polyethylene glycol, polycarboxylated polyethylene glycol, chondroitin-6-sulfate, chondroitin-4-sulfate, keratin, keratin sulfate, heparan sulfate, dermatin, dermatan sulfate, carboxymethylcellulose, heparin, dextran, or hyaluronic acid.

In certain embodiments of the invention, the lipid moiety is either phosphatidic acid, an acyl glycerol, monoacylglycerol, diacylglycerol, triacylglycerol, sphingosine, sphingomyelin, chondroitin-4-sulphate, chondroitin-6-sulphate, ceramide, phosphatidylethanolamine, phosphatidylserine, phosphatidylcholine, phosphatidylinositol, or phosphatidylglycerol, or an ether or alkyl phospholipid derivative thereof, and the physiologically acceptable monomer or polymer moiety is either aspirin, lactobionic acid, maltose, glutaric acid, polyethylene glycol, carboxymethylcellulose, heparin, dextran, hemacell, hetastarch, or hyaluronic acid.

In certain embodiments, the present invention provides for use of a lipid moiety bonded to a physiologically acceptable monomer, dimer, oligomer, or polymer, in the preparation of a pharmaceutical composition for treating a subject afflicted with allergic rhinitis, chronic rhinosinusitis, nasal polyps, asthma, bronchitis, chronic obstructive pulmonary disease, obstructive respiratory disease, colitis, Crohn's disease, central nervous system insult, multiple sclerosis, contact dermatitis, psoriasis, cardiovascular disease, including prophylaxis for invasive procedures, invasive cellular proliferative disorders, anti-oxidant therapy, hemolytic syndromes, sepsis, acute respiratory distress syndrome, tissue transplant rejection syndromes, autoimmune disease, viral infection, and hypersensitivity conjunctivitis.

In certain embodiments, the present invention provides for use of a pharmaceutical composition according to the present invention for treating a subject afflicted with allergic rhinitis, chronic rhinosinusitis, nasal polyps, asthma, bronchitis, chronic obstructive pulmonary disease, obstructive respiratory disease, colitis, Crohn's disease, central nervous system insult, multiple sclerosis, contact dermatitis, psoriasis, cardiovascular disease, including prophylaxis for invasive procedures, invasive cellular proliferative disorders, anti-oxidant therapy, hemolytic syndromes, sepsis, acute respiratory distress syndrome, tissue transplant rejection syndromes, autoimmune disease, viral infection, or hypersensitivity conjunctivitis, wherein the composition is prepared for administration by topical, oral, nasal, aerosol, intravenous, intraocular, intra-arterial, subcutaneous, or suppository routes.

In certain embodiments, the invention provides methods of treating a subject suffering from a disease involving the production and/or action of lipid mediators and/or impairment of glycosaminoglycan (GAG) functioning.

In certain embodiments of the invention, the physiologically acceptable monomer may be, inter alia, a salicylate, salicylic acid, aspirin, a monosaccharide, lactobionic acid, glucoronic acid, maltose, amino acid, glycine, carboxylic acid, acetic acid, butyric acid, dicarboxylic acid, glutaric acid, succinic acid, fatty acid, dodecanoic acid, didodecanoic acid, bile acid, cholic acid, cholesterylhemmisuccinate, or wherein the physiologically acceptable dimer or oligomer may be, inter alia, a dipeptide, a disaccharide, a trisaccharide, an oligosaccharide, an oligopeptide, or a di- or trisaccharide monomer unit of glycosaminoglcans, hyaluronic acid, heparin, heparan sulfate, keratin, keratan sulfate, chondroitin, chondroitin sulfate, chondroitin-4-sulfate, chondroitin-6-sulfate, dermatin, dermatan sulfate, dextran, polygeline, alginate, hydroxyethyl starch, ethylene glycol, or carboxylated ethylene glycol, or wherein the physiologically acceptable polymer may be, inter alia, a glycosaminoglycan, hyaluronic acid, heparin, heparan sulfate, chondroitin, chondroitin sulfate, keratin, keratan sulfate, dermatin, dermatan sulfate, carboxymethylcellulose, dextran, polygeline, alginate, hydroxyethyl starch, polyethylene glycol or polycarboxylated polyethylene glycol.

In some embodiments, the physiologically acceptable polymer may be, inter alia, hyaluronic acid.

In some embodiments, the physiologically acceptable polymer may be, inter alia, chondroitin sulfate.

In certain embodiments of the invention, the lipid or phospholipid moiety may be, inter alia, phosphatidic acid, an acyl glycerol, monoacylglycerol, diacylglycerol, triacylglycerol, sphingosine, sphingomyelin, ceramide, phosphatidylethanolamine, phosphatidylserine, phosphatidylcholine, phosphatidylinositol, phosphatidylglycerol, or an ether or alkyl phospholipid derivative thereof.

In certain embodiments, the phospholipid moiety may be, inter alia, phosphatidylethanolamine.

### Dosages and Routes of Administration

The methods according to certain embodiments of this invention can be adapted to use of the therapeutic compositions comprising Lipid-conjugates in admixture with conventional excipients, i.e. pharmaceutically acceptable organic or inorganic carrier substances suitable for parenteral, enteral (e.g., oral) or topical application which do not deleteriously react with the active compounds. Suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins.

In certain embodiments, pharmaceutical compositions are provided for treating a subject suffering from bronchitis, including a lipid or phospholipid moiety bonded to a physiologically acceptable monomer, dimer, oligomer, or polymer, and a pharmaceutically acceptable carrier or excipient.

In certain embodiments, pharmaceutical compositions are provided for preventing bronchitis in a subject, including a lipid or phospholipid moiety bonded to a physiologically acceptable monomer, dimer, oligomer, or polymer; and a pharmaceutically acceptable carrier or excipient.

In certain embodiments, pharmaceutical compositions are provided for treating a subject suffering from bronchitis, including a lipid or phospholipid moiety bonded to a physiologically acceptable carrier or excipient.

In certain embodiments, pharmaceutical compositions are for preventing bronchitis in a subject, including a lipid or phospholipid moiety bonded to a physiologically acceptable carrier or excipient.

In certain embodiments, pharmaceutical compositions are provided for treating a subject suffering from bronchitis, including any one of the compounds according to the invention or any combination thereof; and a pharmaceutically acceptable carrier or excipient. In certain embodiments, pharmaceutical compositions are provided for preventing bronchitis in a subject, including any one of the compounds according to the invention or any combination thereof; and a pharmaceutically acceptable carrier or excipient.

In certain embodiments, the compounds according to the invention include, inter alia, the compounds represented by the structures of the general formulae: (A), (I), (II), (III), (IV), (V), (VI), (VII), (VIII), (IX), (IXa), (IXb), (X), (XI), (XI), (XIII), (XIV), (XV), (XVI), (XVII), (XVIII), (XIX), (XX), (XXI), (XXII) or any combination thereof.

While the examples provided herein describe use of the PL conjugates in subcutaneous, intraperitoneal or topical administration, the success described affords good evidence to suppose that other routes of administration, or combinations with other pharmaceutical preparations, would be at least as successful. The route of administration (e.g., topical, parenteral, enteral, intravenous, vaginal, inhalation, nasal aspiration (spray), suppository or oral) and the dosage regimen will be determined by skilled clinicians, based on factors such as exact nature of the condition being treated, the severity of the condition, the age and general physical condition of the patient, and so on.

In general, the doses utilized for the above described purposes will vary, but will be in an effective amount to exert the desired anti-disease effect. As used herein, the term “pharmaceutically effective amount” refers to an amount of a compound of formulae A and I-XXI which will produce the desired alleviation in symptoms or signs of disease in a patient. The doses utilized for any of the above-described purposes will generally be from 1 to about 1000 milligrams per kilogram of body weight (mg/kg), administered one to four times per day, or by continuous IV infusion. When the compositions are dosed topically, they will generally be in a concentration range of from 0.1 to about 10% w/v, administered 1-4 times per day.

As used herein, the term “pharmaceutically acceptable carrier” refers to any formulation which is safe, and provides the appropriate delivery for the desired route of administration of an effective amount of at least one compound of the present invention. As such, all of the above-described formulations of the present invention are hereby referred to as “pharmaceutically acceptable carriers.” This term refers to as well the use of buffered formulations wherein the pH is maintained at a particular desired value, ranging from pH 4.0 to pH 9.0, in accordance with the stability of the compounds and route of administration.

For parenteral application, particularly suitable are injectable, sterile solutions, preferably oily or aqueous solutions, as well as suspensions, emulsions, or implants, including suppositories. Ampoules are convenient unit dosages.

For application by inhalation, particularly for treatment of airway obstruction or congestion, solutions or suspensions of the compounds mixed and aerosolized or nebulized in the presence of the appropriate carrier.

For topical application, particularly for the treatment of skin diseases such as contact dermatitis or psoriasis, admixture of the compounds with conventional creams or delayed release patches is acceptable.

For enteral application, particularly suitable are tablets, dragees, liquids, drops, suppositories, or capsules. A syrup, elixir, or the like can be used when a sweetened vehicle is employed. When indicated, suppositories or enema formulations may be the recommended route of administration.

Sustained or directed release compositions can be formulated, e.g., liposomes or those wherein the active compound is protected with differentially degradable coatings, e.g., by microencapsulation, multiple coatings, etc. It is also possible to freeze-dry the new compounds and use the lyophilisates obtained, for example, for the preparation of products for injection.

Thus, embodiments of the present invention provides for use of the Lipid-conjugates in various dosage forms suitable for nasal, aerosol, rectal, vaginal, conjunctival, intravenous, intra-arterial, and sublingual routes of administration.

It will be appreciated that the actual preferred amounts of active compound in a specific case will vary according to the specific compound being utilized, the particular compositions formulated, the mode of application, and the particular situs and organism being treated. Dosages for a given host can be determined using conventional considerations, e.g., by customary comparison of the differential activities of the subject compounds and of a known agent, e.g., by means of an appropriate, conventional pharmacological protocol.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

The main abbreviations used in the application are:

HA=hyaluronic acid

HYPE=dipalmitoyl-phosphatidyl-ethanolamine (PE) conjugated to HA (also referred to as HyPE, HyalPE)

CSA=chondroitin sulfate A

CSAPE=PE conjugated to CSA (also referred to as CsAPE, CsaPE)

CMC=carboxymethyl cellulose

CMPE=PE conjugated to CMC

HEPPE=PE conjugated to heparin (also referred to as HepPE, HePPE)

DEXPE=PE conjugated to dextran

AsPE=PE conjugates to aspirin

HemPE=PE conjugated to Polygeline (haemaccel)

HyDMPE=dimyristoyl PE linked to HA.

Examples demonstrating the utility of lipid-conjugates in preventing and treating disease are presented in PCT/US05/06591 filed 2 Mar. 2005, U.S. application Ser. No. 10/989,606 filed 17 Nov. 2004 and U.S. application Ser. No. 10/989,607 filed 17 Nov. 2004, which are incorporated herein by reference in their entirety.

### Example 1

### A Two-Arm Study to Examine the Safety, Tolerability, and Efficacy of Multiple Intranasal Doses of HyPE on the Response to Nasal Antigen in Allergic Rhinitis Participants Outside of the Allergy Season

Overall Study Design

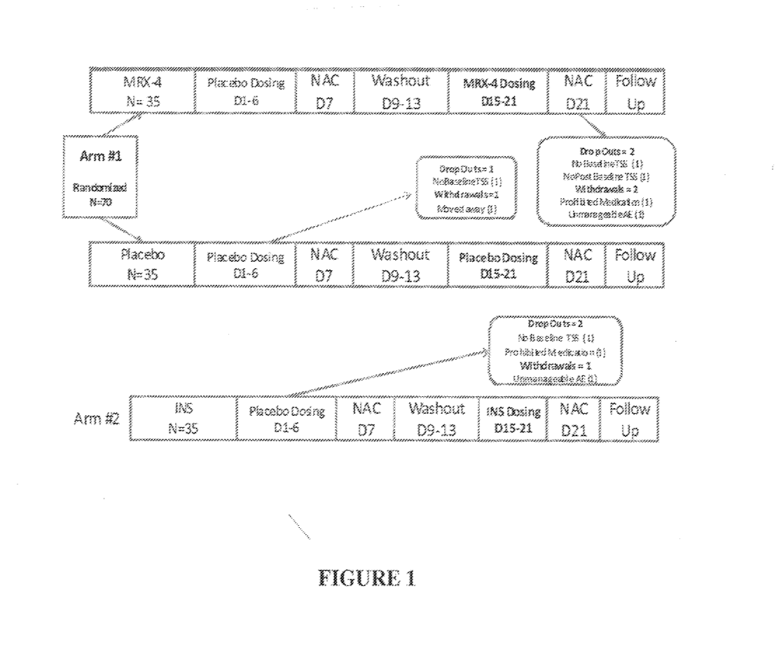

Described in this example is a Phase 2, single center, 2-armed study in participants with allergic rhinitis (AR) (See FIG. 1). Participants in Arm 1 were enrolled in a double-blind, placebo-controlled, randomized study evaluating the safety, tolerability, and efficacy of 2% HyPE (Drug or “MRX-4”) when administered intra-nasally BID for 6 days. Participants in Arm 2 underwent the same procedures and treatment regime, but received an intranasal steroid in a single-blind fashion. Participants in both arms were blinded at all times to their treatment assignment. All participants underwent a placebo lead-in period (Days 1 to 7) prior to receiving their assigned treatment.

A placebo group was included in Arm 1 (a vehicle composed of isotonic PBS with benzyl alcohol as the preservative) to control for environmental changes (pollen). An intranasal corticosteroid arm (INS) (single blinded) was included to provide a positive control for prevention of symptoms, nasal inflammation and mediator release following NAC (Nasaal Antigen Challenge). Drug and placebo were given BID, at approximately 8:00 am and 8:00 pm, using a multiple dose nasal applicator. Eligible participants were randomized in a 1:1 ratio (Drug:Placebo) in the double-blind portion of the study. Once enrolment in Arm 1 had been completed (70 participants had been enrolled), 35 participants were enrolled in Arm 2 (Table 1.1).

TABLE 1.1
Treatment Groups

| Treatment Group | | Days 1 to 7 | Days 15 to 21 |
|---|---|---|---|
| Arm 1 | Group 1 | Placebo (Vehicle) | Drug (2.0% HyPE) |
| | (n = 35) | | |
| | Group 2 | Placebo (Vehicle) | Placebo (a vehicle |
| | (n = 35) | | composed of isotonic PBS |
| | | | with benzyl alcohol as the |
| | | | preservative) |
| Arm 2 | (Group 3, | Placebo (Vehicle) | Steroid (Rhinocort |
| | n = 35) | | [budesonide], 32 μg) |

Indication and Main Criteria for Inclusion:

Healthy adult males and females between 18 and 65 years old, with a history of summer grass pollen allergic rhinitis for at least 2 years, confirmed by a positive skin prick test to Bermuda or Rye grass pollen extract, defined as a 23 mm wheal compared with the negative control. Participants must not have used any oral or intranasal, prescription or over-the-counter, anti-allergy medication within the previous 4 weeks or immunotherapy in the previous 3 months. Participants were studied outside of the grass pollen season.

Treatments Administered

HyPE for intranasal administration was provided in an intranasal spray bottle suitable for the administration of multiple doses over the treatment period. Placebo (a vehicle composed of isotonic PBS with benzyl alcohol as the preservative) was provided in a matching multi-dose intranasal spray for the double-blind portion of the study. The steroid used was commercially-available INS, budesonide aqueous spray (RhinocortS, AstraZeneca)

HyPE was administered intranasally as a 2% HyPE concentration in phosphate-buffered saline (PBS) with benzyl alcohol as a preservative. The solution was placed in glass bottles and closed with the Valois Equadel nasal spray device. Each activation of the nasal applicator delivered 100 μL of solution; resulting in a total dose of 200 μL (1 spray in each nostril) to provide 4 mcg. Rhinocort was administered intra-nasally 2 sprays BID at a dose of 32 μg per spray (total daily dose 256 μg/day)

Duration of Treatment:

The study consisted of 2 treatment weeks, separated by 1 week of wash-out. Including up to 6 weeks of screening and 4 weeks of follow-up, the entire study could last up to approximately 12 weeks. Participants were required to visit the clinic up to 8 times, including 2 full days (as an outpatient) during which they had the NAC followed by nasal lavage procedures.

All treatments were self-administered. In Arm 1, Drug and placebo were administered using the same type of intranasal applicator, which provided 100 μL of solution to each nostril BID, resulting in a 200 μL dose BID. In Arm 2. Rhinocort 32 μg was administered as 2 sprays BID. The timing of the treatments were as follows (FIG. 1):

Days 1 to 7 (placebo run-in): all participants received placebo (isotonic strength PBS with benzyl alcohol) BID at approximately 8:00 am and 8:00 pm.

Days 8 to 14: washout period.

Days 15 to 21: participants received either Drug, INS or placebo BID at approximately 8:00 am and 8:00 pm.

Nasal Lavage

A subset of participants in each treatment group was selected to undergo nasal lavage procedures for the collection of inflammatory mediators from the nose. Nasal lavage was conducted using 6 mL of warm (37° C.) PBS using a 10 mL syringe attached by tubing to a nasal adaptor or olive. Participants were seated in a forward-flexed neck position (60° from the upright) to prevent fluid from reaching the nasopharynx. To ensure adequate washing, the lavage fluid was passed slowly into the nasal cavity and then left to dwell for 30 seconds. The fluid was then flushed and withdrawn back into the syringe approximately 30 times in 2 minutes until turbid.

The levels of each inflammatory mediator measured in the nasal lavage fliud (leukocytes, eosinophils, cytokines and chemokines) and changes from baseline were examined at each time point.

Nasal Antigen Challenge

The Nasal Antigen Challenge (NAC) was performed by administering Bermuda or Rye grass pollen into the nasal cavity as two 100 μL doses using the BiDose applicator. The dose was to defined at screening as the lowest concentration which elicited a positive reaction during a previously administered skin prick test.

Criteria for Evaluation:

Safety:

Safety variables were summarized for each dose level and overall. Safety variables included adverse events (AEs), laboratory tests, vital signs (oral body temperature, systolic and diastolic blood pressures, pulse, and respiratory rate), electrocardiogram results, physical examination findings, and concomitant medications.

Pharmacokinetics:

Blood samples for the pharmacokinetic assessment of the serum levels of HyPE were collected on Day 14 (baseline) and on Day 21, immediately after administration of study drug.

Efficacy:

Nasal symptoms were recorded at 0, 0.5, 1.5, 2.5, 4.5, 6.5, 8.5, and 24 hours postdose on Days 7 and 21. Symptoms of nasal congestion, rhinorrhea, frontal headache, post-nasal drip, sneezing, nasal itch, itching ears/palate and cough were each scored on a scale from 0 to 3 (0=none, 1-mild, 2=moderate, 3=severe symptoms). The primary efficacy endpoint was the total symptom score (TSS) comprised of the following 4 symptoms: nasal congestion, rhinorrhea, sneezing and nasal itch. Thus the TSS at each time point ranged from 0 (no symptoms) to 12 (maximal symptoms). The change in the mean symptom score over each 24 hour period postdose from Day 7 (baseline) and Day 21 (post-treatment) were compared among treatment groups as well as changes in clinical improvement as measured by population shift.

Secondary efficacy endpoints included change from baseline in the mean of each of the 8 individual nasal symptoms over 24 hours. In addition, the levels of each inflammatory mediators (leukocytes, eosinophils, cytokines and chemokines) and changes from baseline were examined at each timepoint in those participants who underwent nasal lavage.

Selection of Allergen Responders During NAC (Post Study Analysis).

Since the primary efficacy analysis was the comparison of TSS after NAC after 6 days of daily dosing with the Drug and placebo, and the fact that, in spite of careful selection of participants and of dose of antigen used during NAC, some participants failed to develop nasal symptoms, making evaluation of the Drug impossible. Accordingly, for the purpose of evaluating efficacy, only those participants that experienced significant symptom levels following NAC at Day 7 (after 6 days of placebo treatment) were included in the primary efficacy analysis.

Participants were selected if at Day 7 (baseline) they reported at least one “moderate” score (≧2) at any time point between Ohrs and 24 hrs (0 h, 0.5 h, 1.5 h, 2.5 h, 4.5 h, 6.5 h, 8.5 h and 24 h) in at leas two of the four clinical categories recorded (nasal congestion, rhinorrhea, sneezing and nasal itch). This generated a sub-population of participants for each of the study arms.

Clinical improvement was determined by population shift analysis: The difference (shift) in the number of participants exhibiting an allergic response on Day 21 versus Day 7 for each treatment arm. This difference may be attributed to the treatment since non-responders had not been included in the analysis.

Adverse Events

The most total, as well as selected specific, number and percentage, of Treatment Emergent Adverse Events (TEAEs) are presented in the Table 1.2.

TABLE 2.2
Treatment Emergent Adverse Events:

| | | Placebo | Drug | INS |
|---|---|---|---|---|
| | | (N = 35) | (N = 35) | (N = 35) |
| Total number of | | 16 (46%) | 14 (40%) | 19 (54%) |
| participants | | | | |
| recording a TEAE | | | | |
| Infections and | Total | 13 (37%) | 9 (26%) | 10 (29%) |
| infestations | Rhinitis | 10 (29%) | 7 (20%) | 8 (23%) |
| | Upper Respi- | 1 (3%) | 2 (6%) | 2 (6%) |
| | ratory Tract | | | |
| | Infection | | | |
| Respiratory, | Total | 6 (17%), | 4 (11%) | 9 (26%) |
| Thoracic | Cough | 4 (11%) | 0 | 0 |
| And Mediastinal | Postnasal Drip | 2 (6%) | 2 (6%) | 0 |
| Disorders* | Sneezing | 2 (6%) | 1 (3%) | 1 (3%) |
| Nervous System | Total | 4 (11%) | 5 (14%) | 9 (26%) |
| Disorders | Headache | 4 (11%) | 2 (6%) | 6 (17%) |

2% HyPE given intranasally for 6 days had similar safety and tolerability to placebo with the exception of 2 dropouts (due to low platelet count and forbidden concomitant medication Interesting observations about 2% HyPE when administered intra-nasally in this study included: (i) decreased cough, (ii) decreased headache, and (iii) decreased need for an asmtha rescue medication (e.g., salbutamol), relative to placebo and comparable to intransal steroid treatment.

Efficacy Results

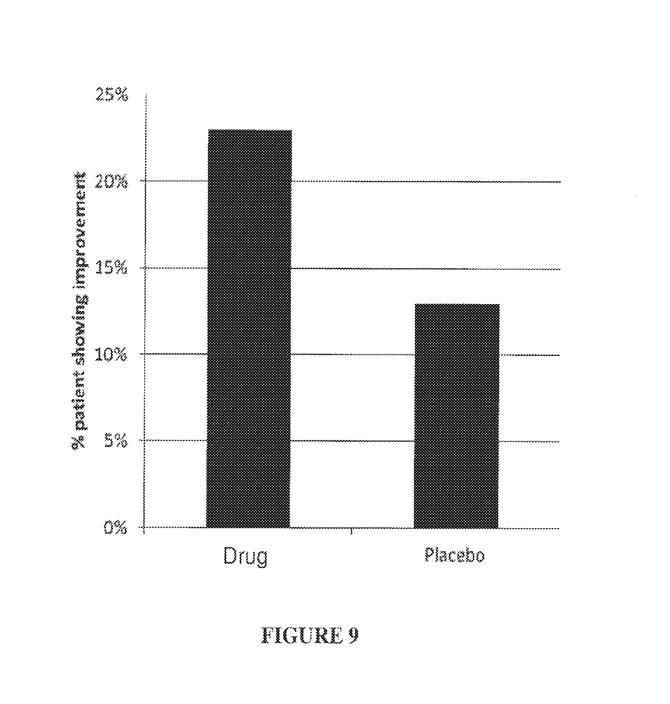

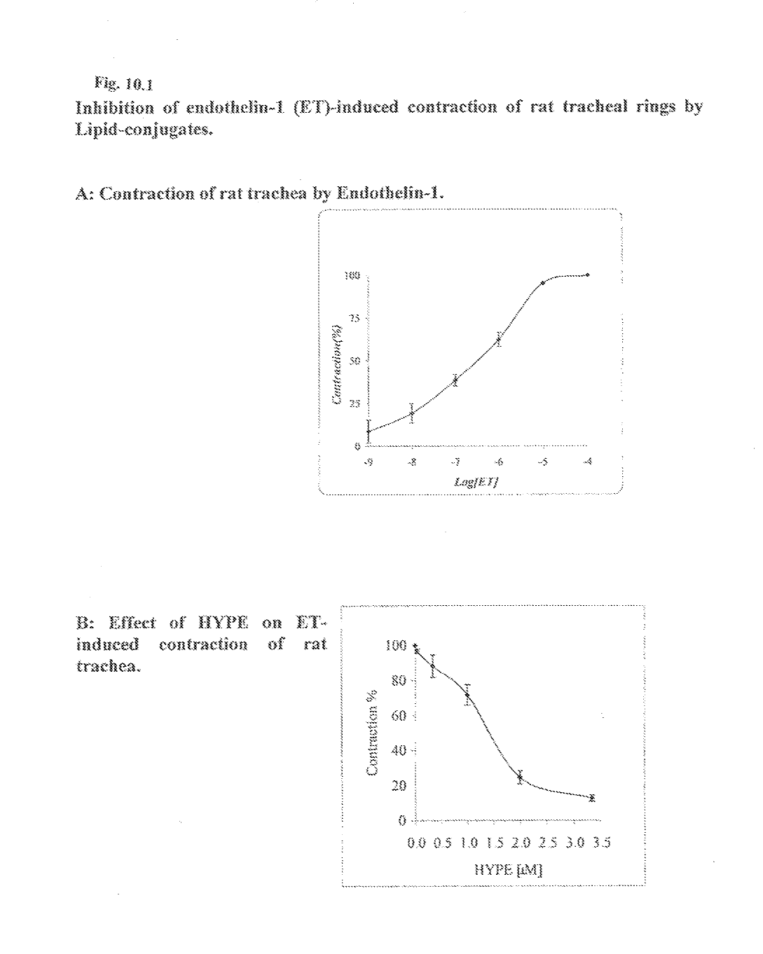

The primary endpoint was reported for the allergen responder sub-population. Summaries and analysis relating to the clinical improvement (population shift) are presented in Table 1.3 to below and FIG. 9.

TABLE 1.3
Clinical efficacy (population shift) Add n and percentage
responders in each of the first two columns

| | Allergen responders | | | |
|---|---|---|---|---|
| | on Day 7 | Allergen responders | | |
| | (% out of FAS) | on Day 21 | | |
| | [n = number of | (%\of | | |
| | participants that | allergen | Differ- | Differ- |
| | responded to | responders | ence | ence |
| Group | NAC on day 7] | at Day 7) | (delta n) | in % |
| Control | 26 (73%) | 23 (89%) | 3 | (11%) |
| (Day 7 | | | | |
| placebo, | | | | |
| Day 21 | | | | |
| placebo) | | | | |
| Drug | 28 (80%) | 22 (79%) | 6 | (21%) |
| (Day 7 | | | | |
| placebo, | | | | |
| Day 21 | | | | |
| Drug) | | | | |
| Steroid | 23 (66%) | 10 (44%) | 13 | (56%) |
| (Day 7 | | | | |
| placebo, | | | | |
| Day 21 | | | | |
| steroid) | | | | |

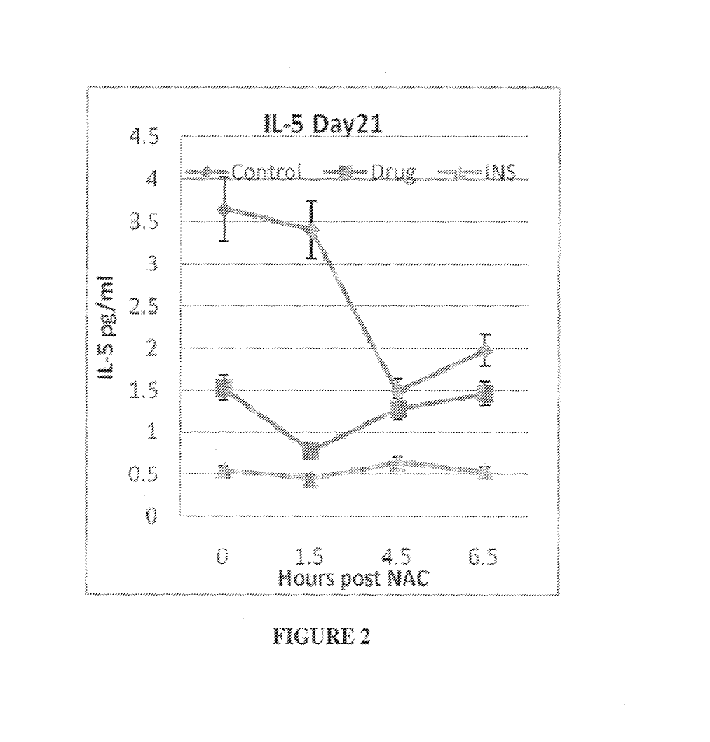

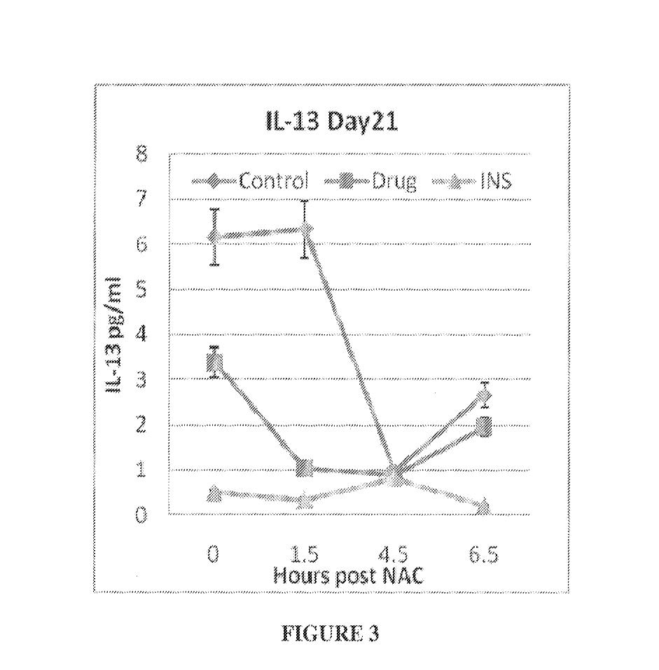

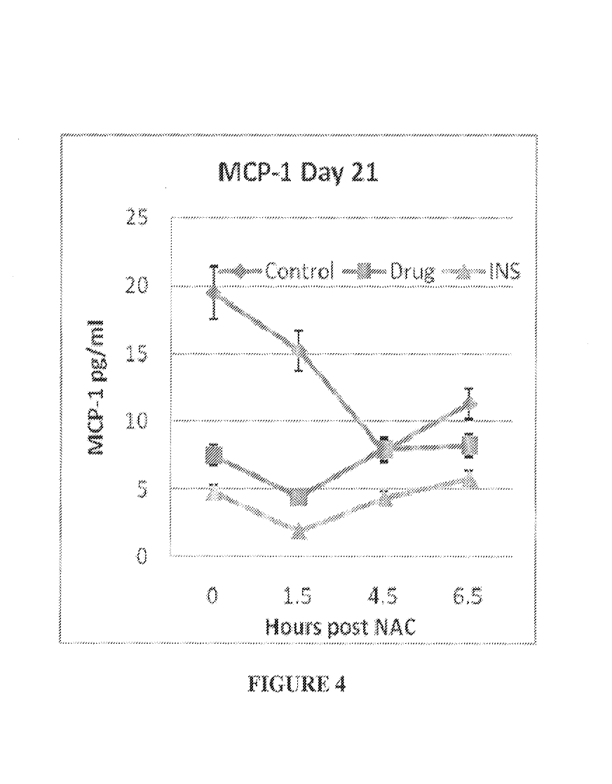

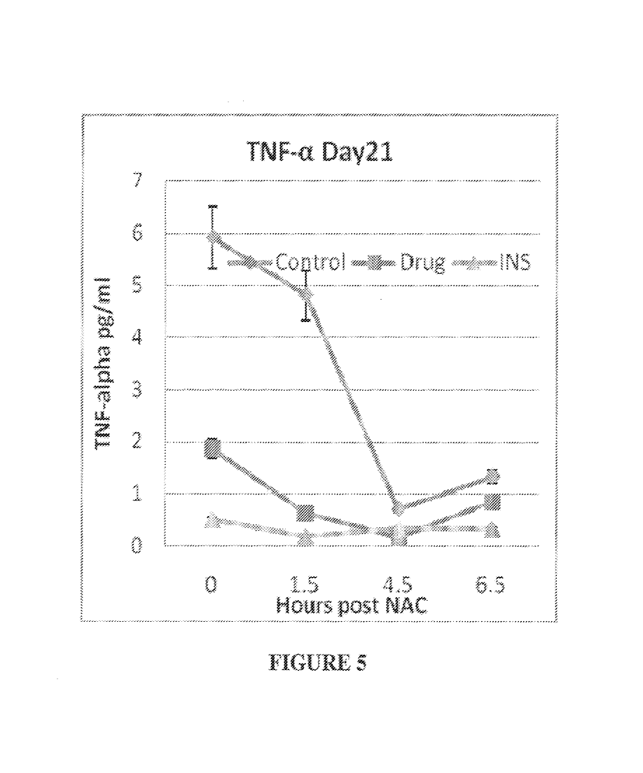

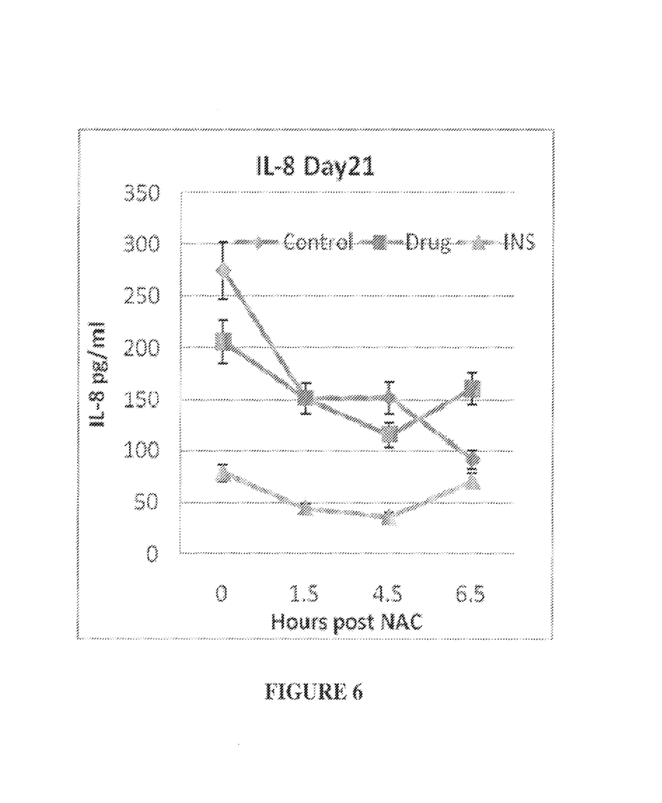

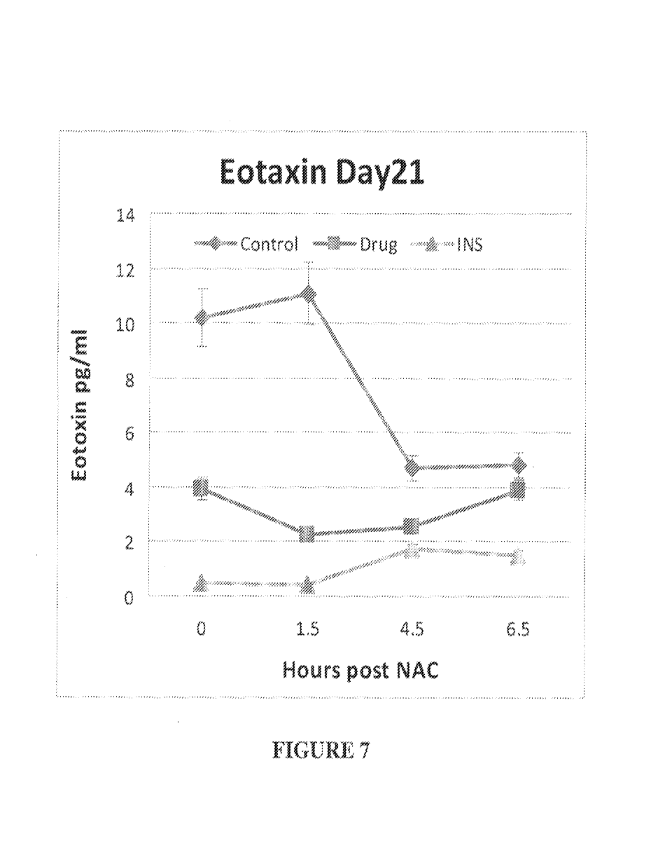

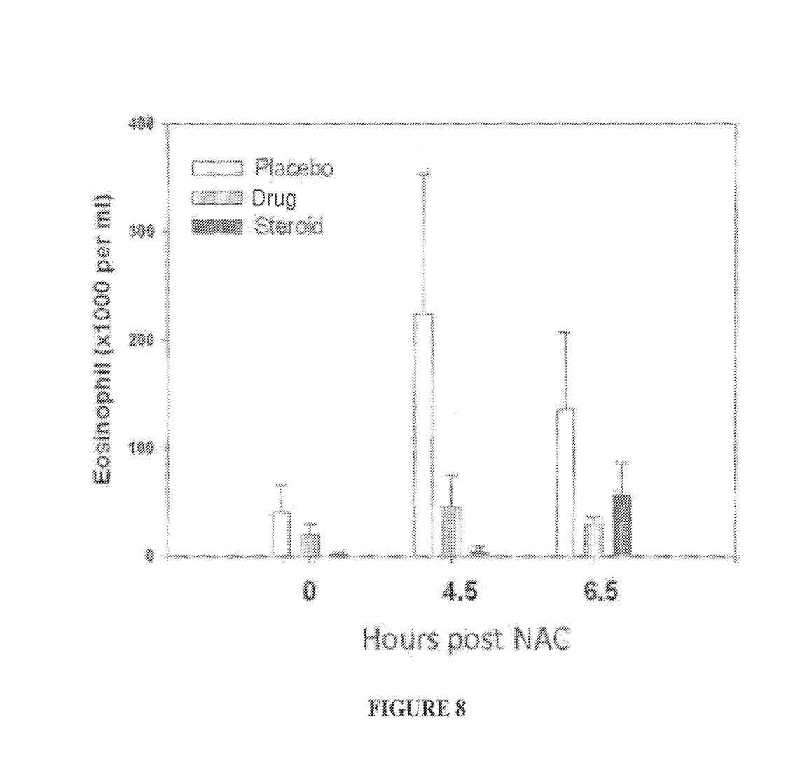

Plots of the mean (normalised) cytokine levels, IL-5, IL-13, MCP-1, TNF-α, IL-8 and eotaxin, and (normalised) cosinophils at Day 21 for the Placebo, MRX-4 and steroid groups are presented in FIGS. 2-8 respectively.

In conclusion, six days of intranasal treatment with 2% HyPE administered intranasally had similar safety and tolerability to placebo with the exception of 2 dropouts (due to low platelet count and forbidden concomitant medication). Furthermore, efficacy analyses showed symptom improvement relative to placebo and approaching intranasal steroid for selected symptoms and inflammatory mediators.

### Example 3

### Obstructive Respiratory Disease

The Lipid-conjugates are effective in the treatment of obstructive respiratory disease. This is demonstrated for asthma in the Experiments below. In asthma, the impeded airflow is due to airway obstruction which is the result of constriction and obstruction of luminal vessels of the lungs. One widely-accepted experimental system to investigate airway constriction is to induce smooth muscle preparations, isolated from airways, to contract in the absence and presence of the drug. Another widely-accepted test of anti-asthma drug action is to use live animals which have asthma. This disease is present in animals which have been sensitized to an antigen and which can be monitored for exacerbation and recovery from asthmatic breathing using a body plethysmography.

In Experiments 3.1-3.3, the muscle preparation (tracheal rings) was isolated from rats and in Experiment 3.4-3.5 from guinea pigs. Muscle contraction is measured by attachment of the muscle to a pressure transducer, which works much like a spring. Induction of contraction occurs when asthmatogenic substances are administered such as endothelin-1 (ET) an acetylcholine (AcCh).

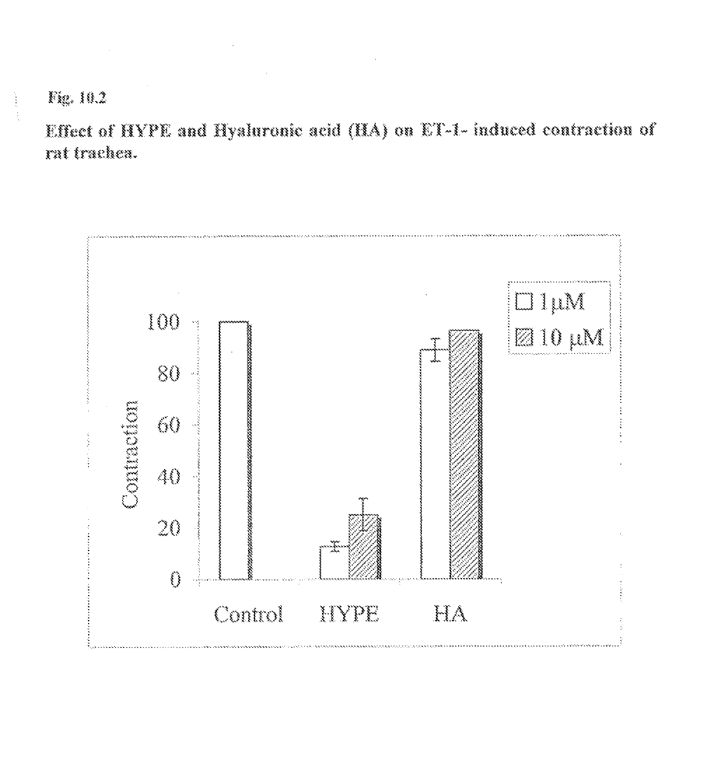

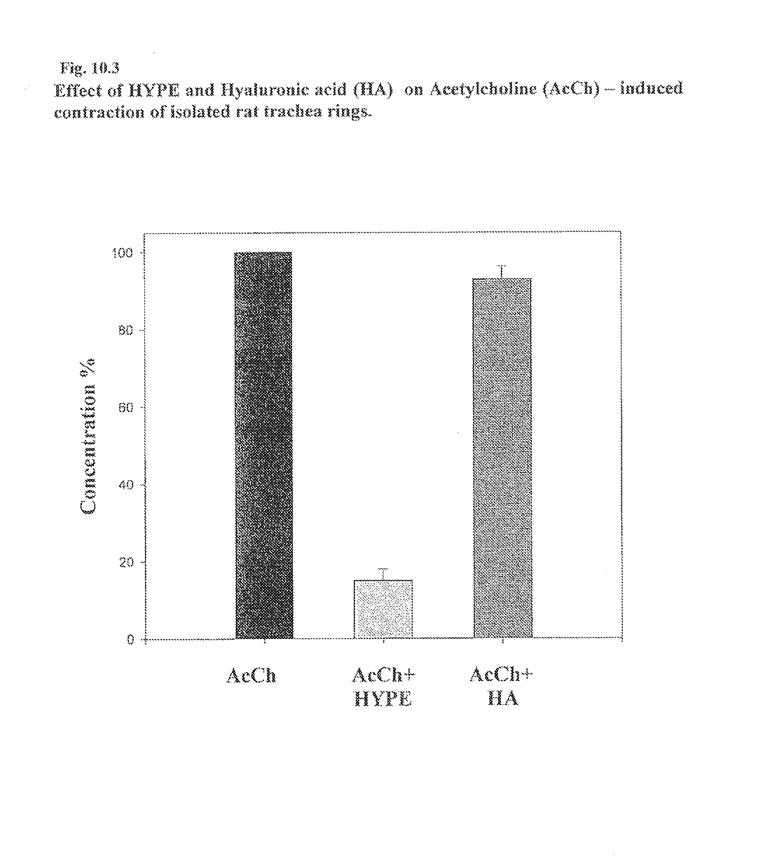

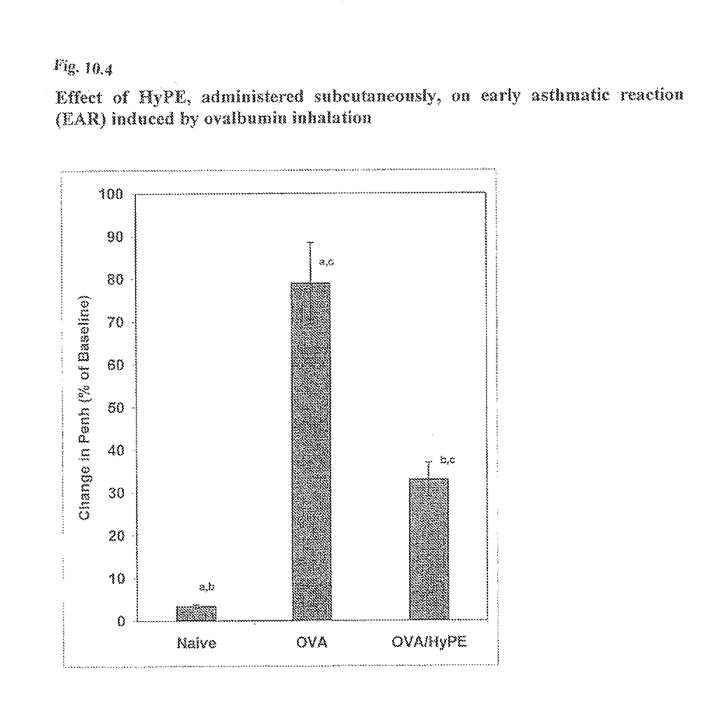

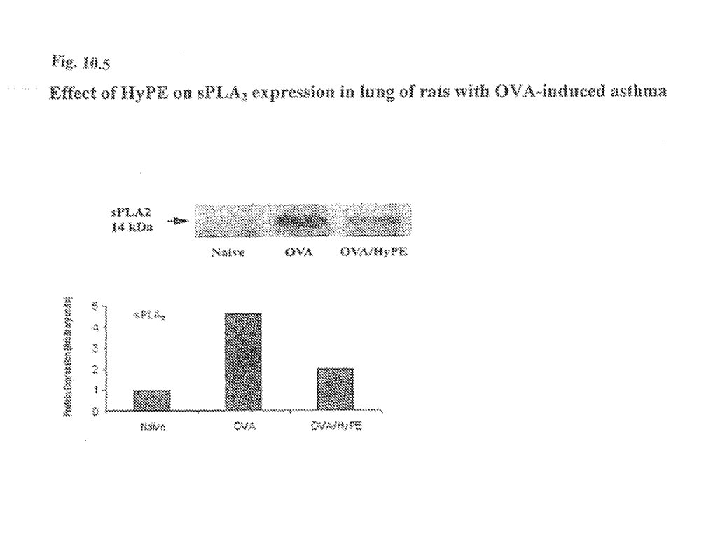

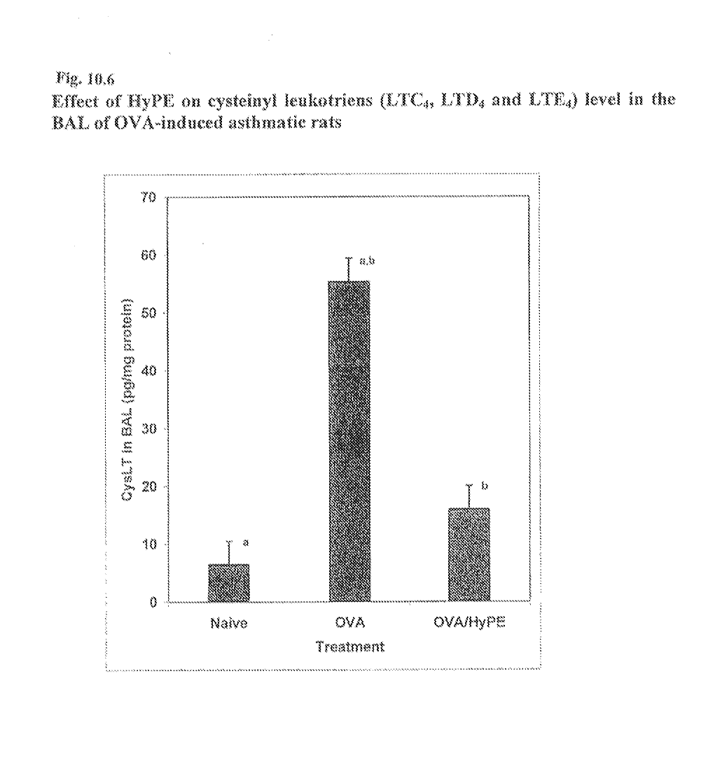

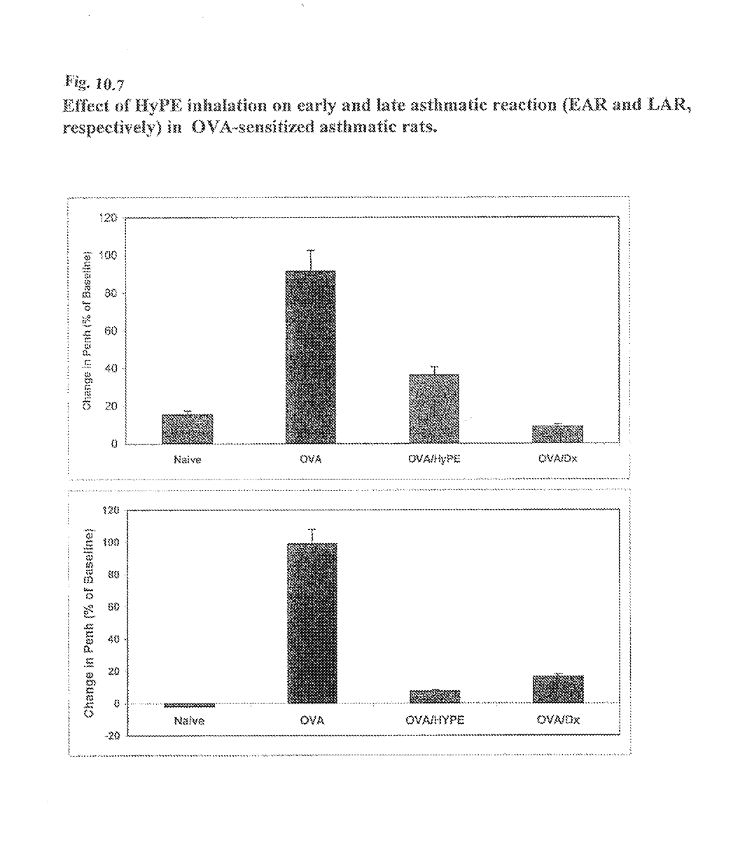

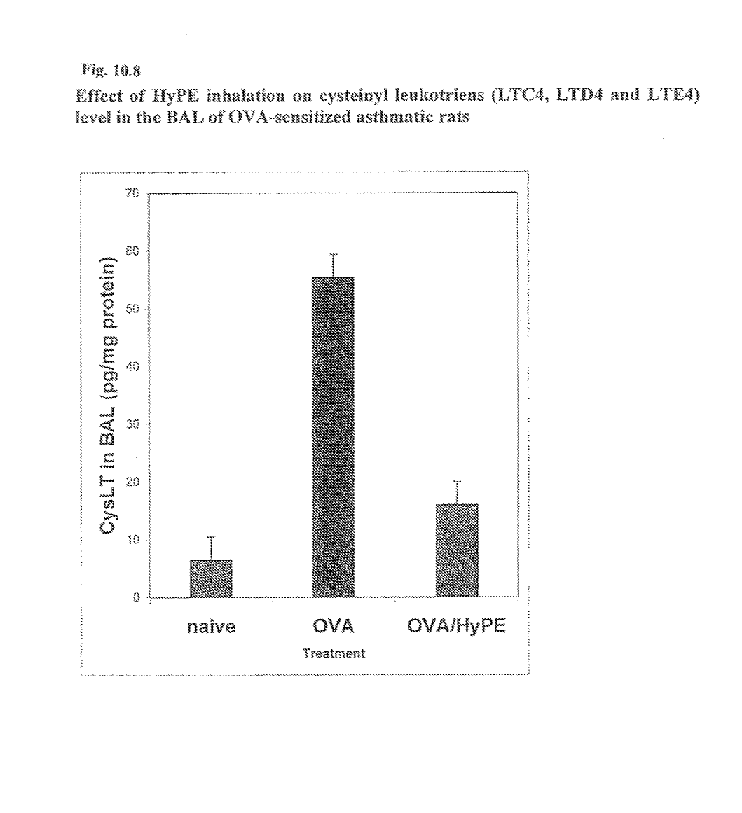

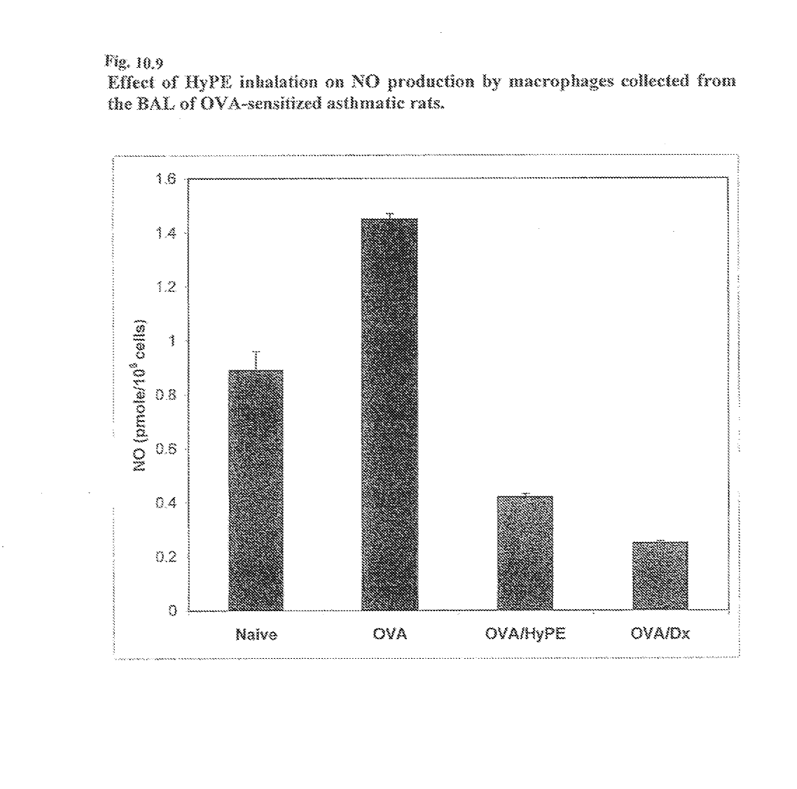

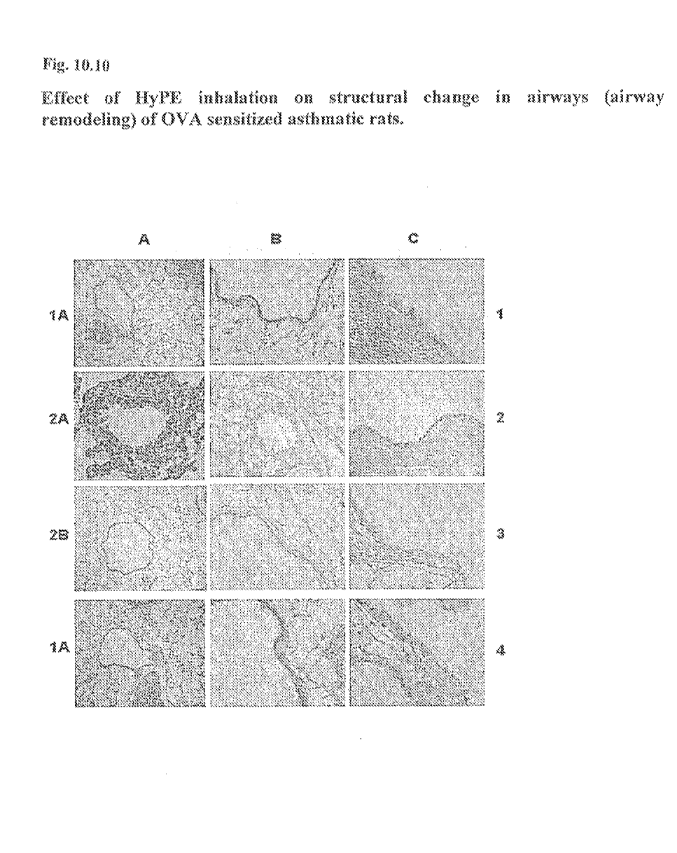

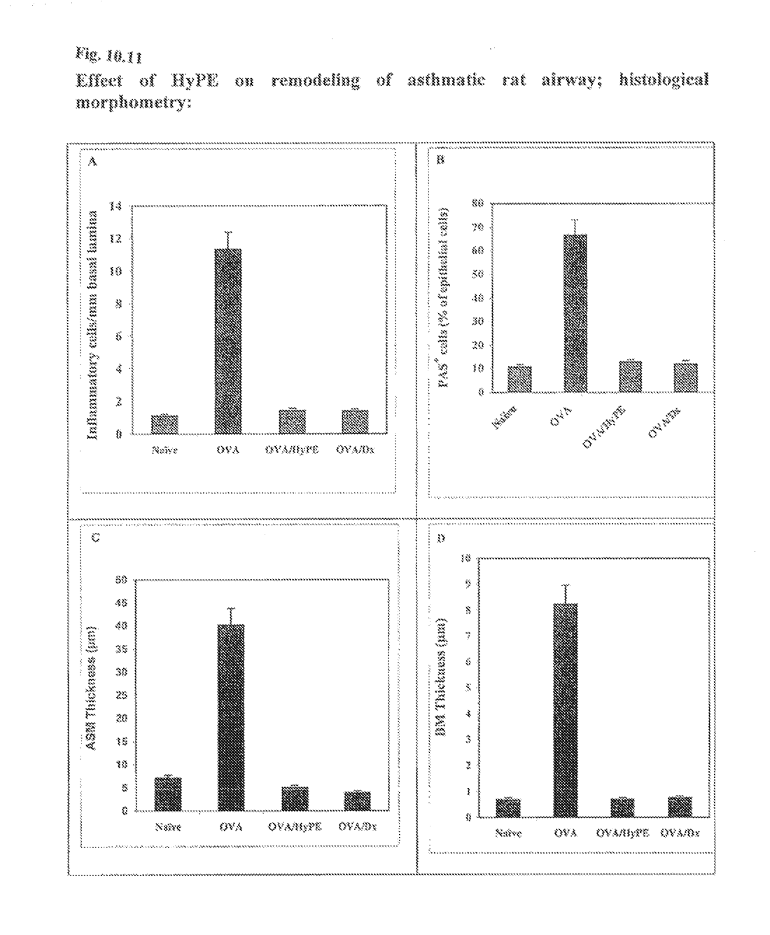

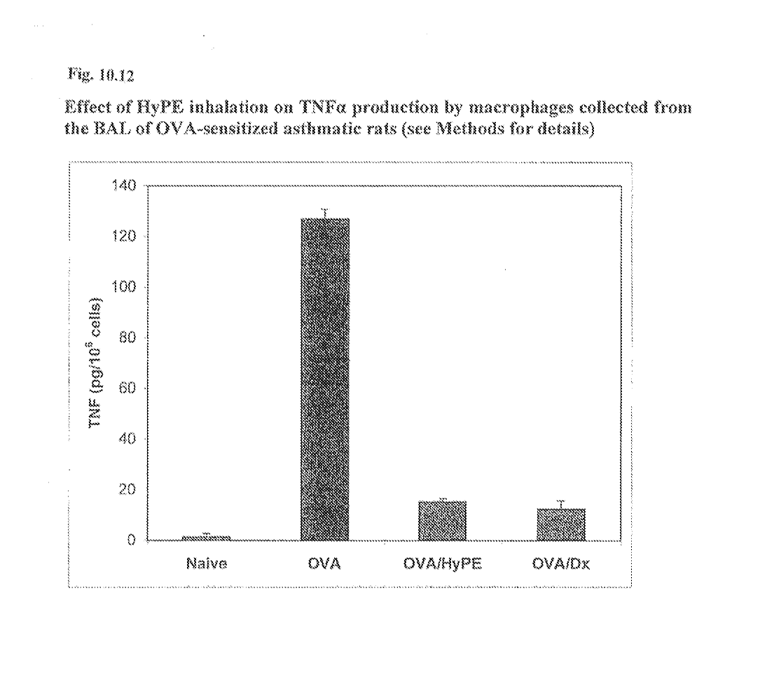

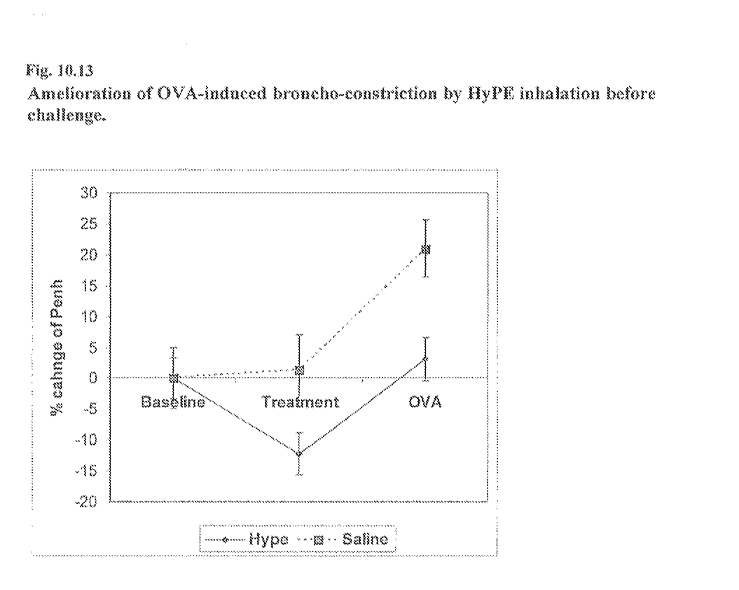

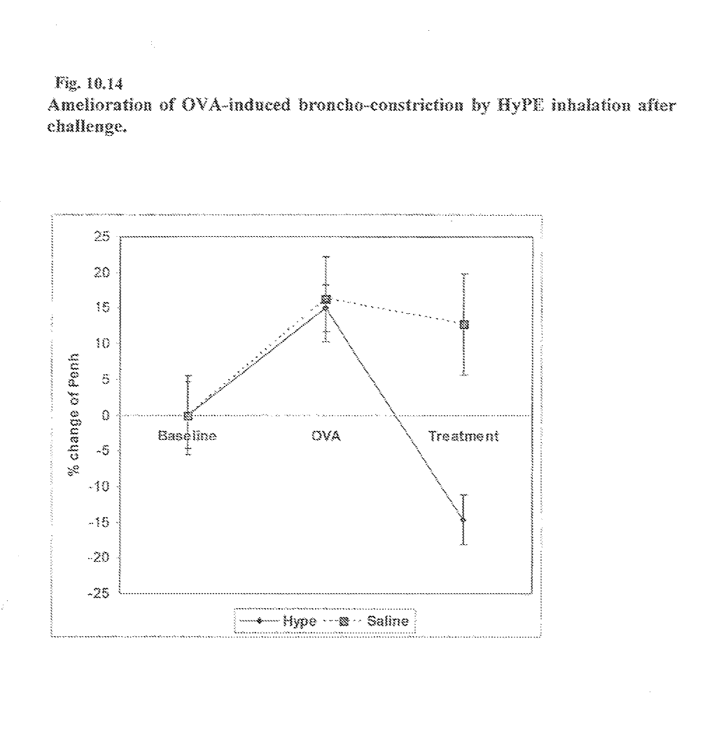

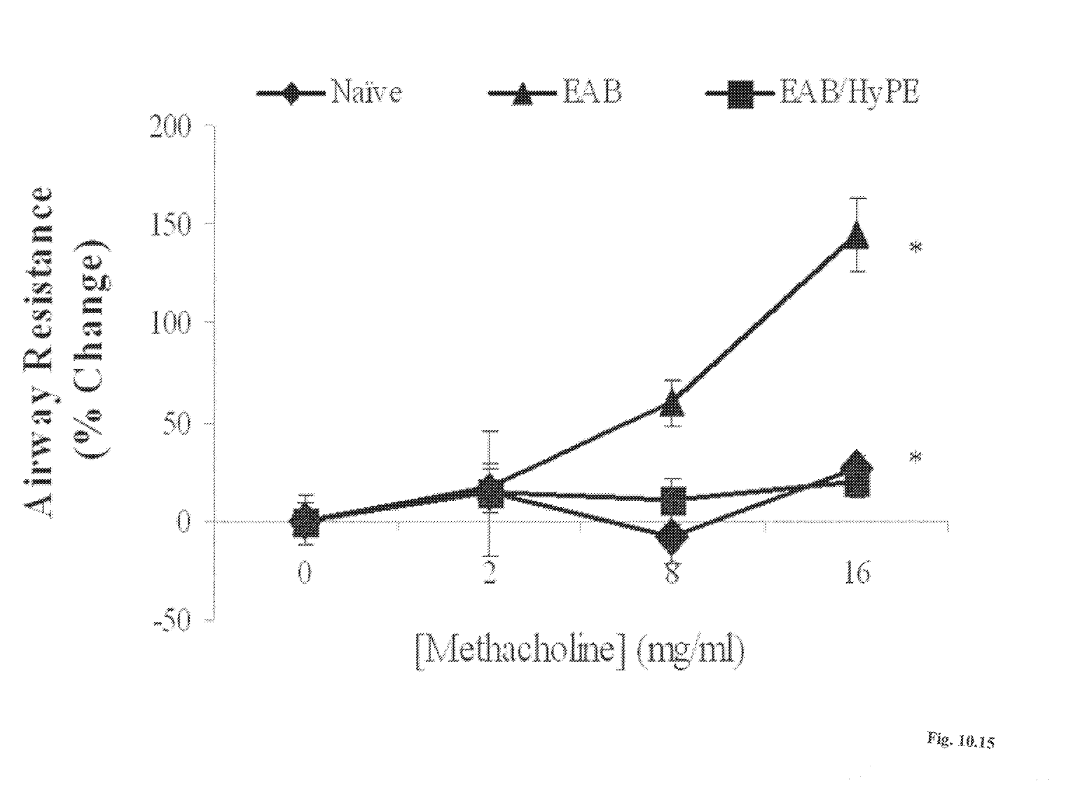

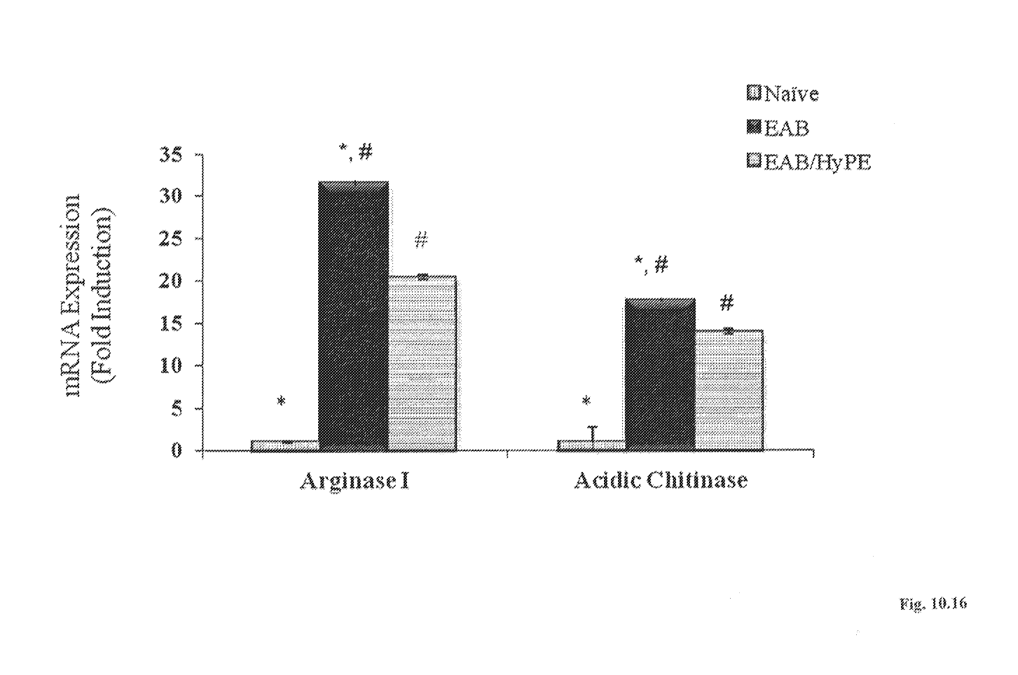

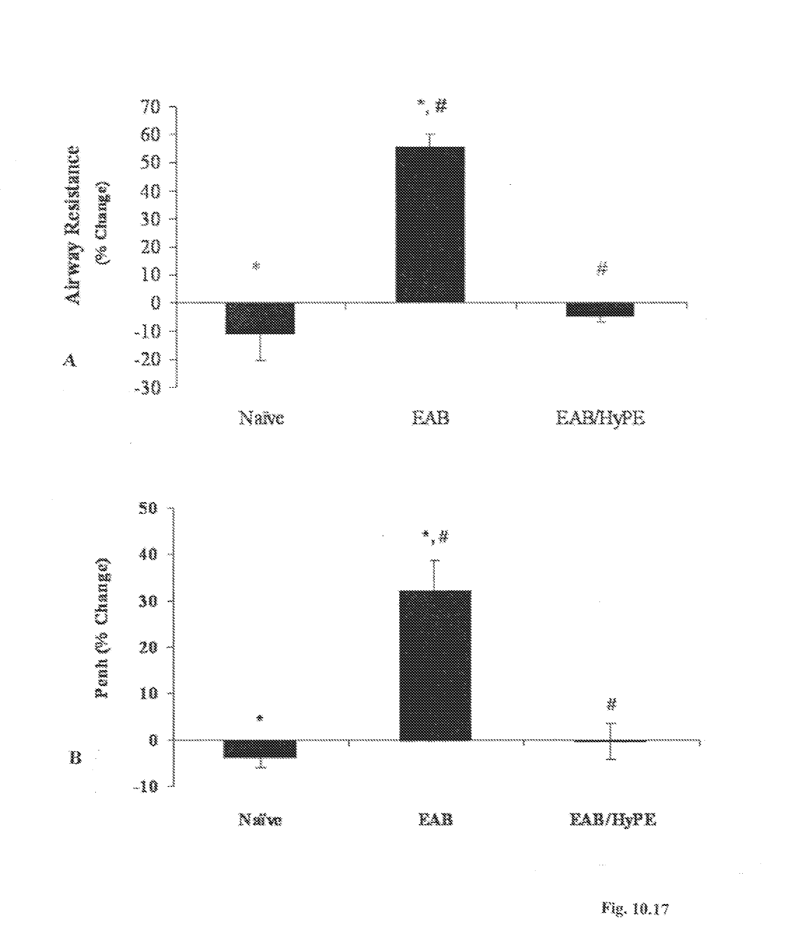

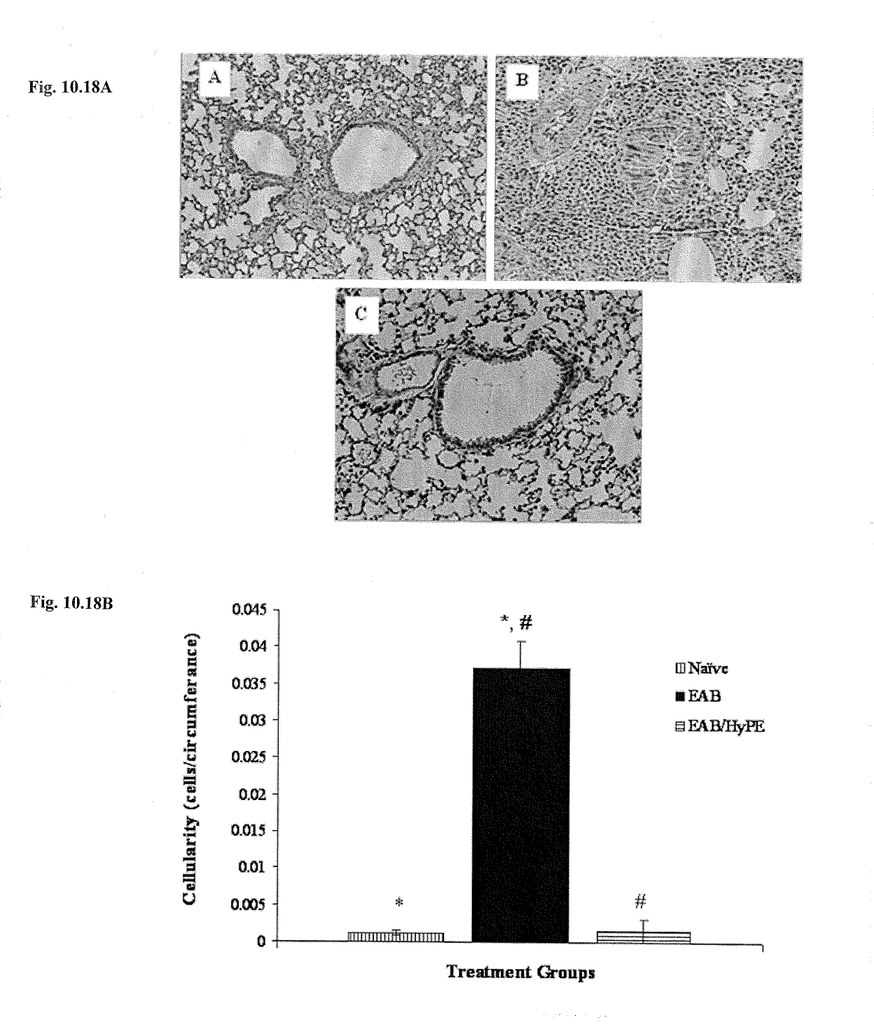

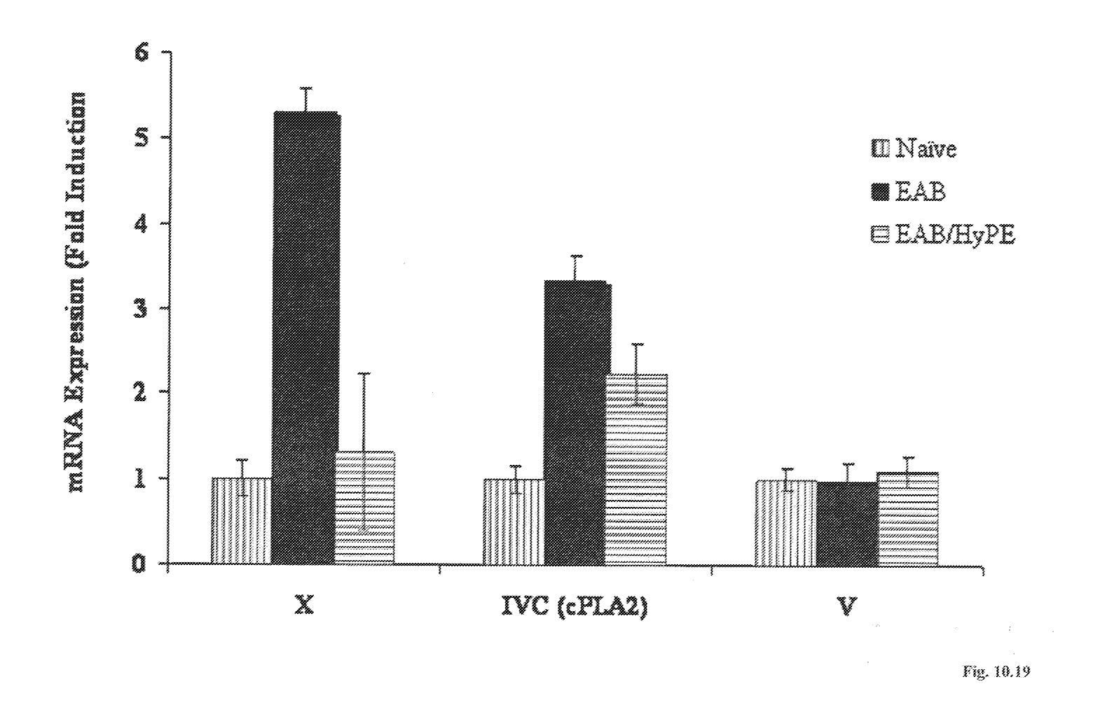

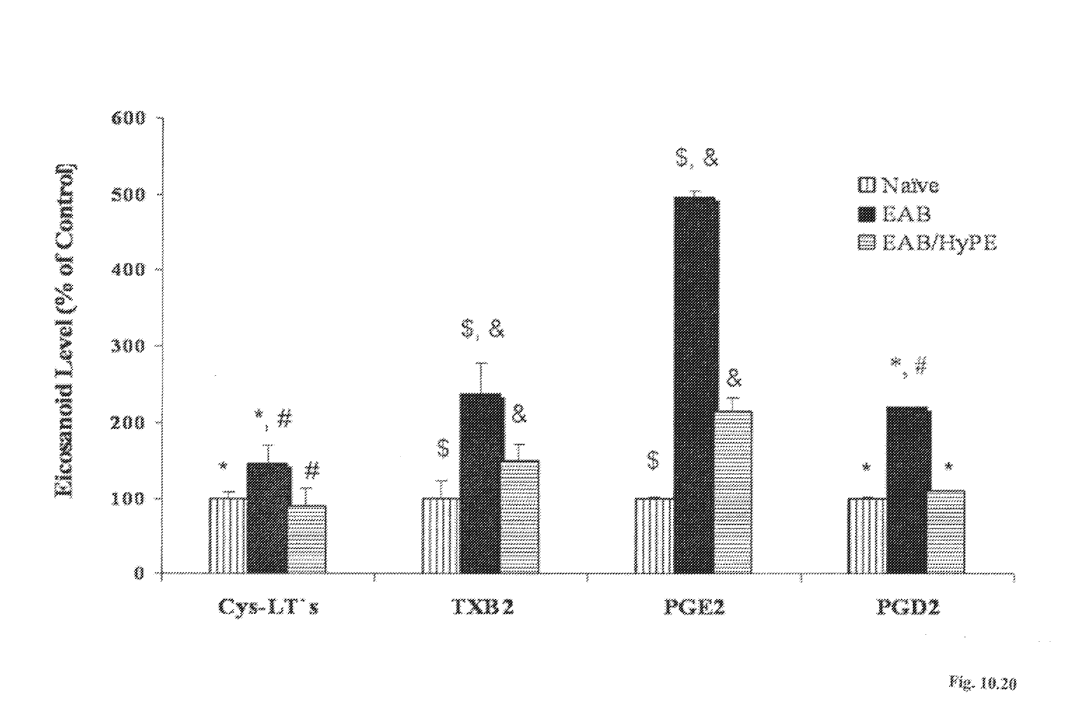

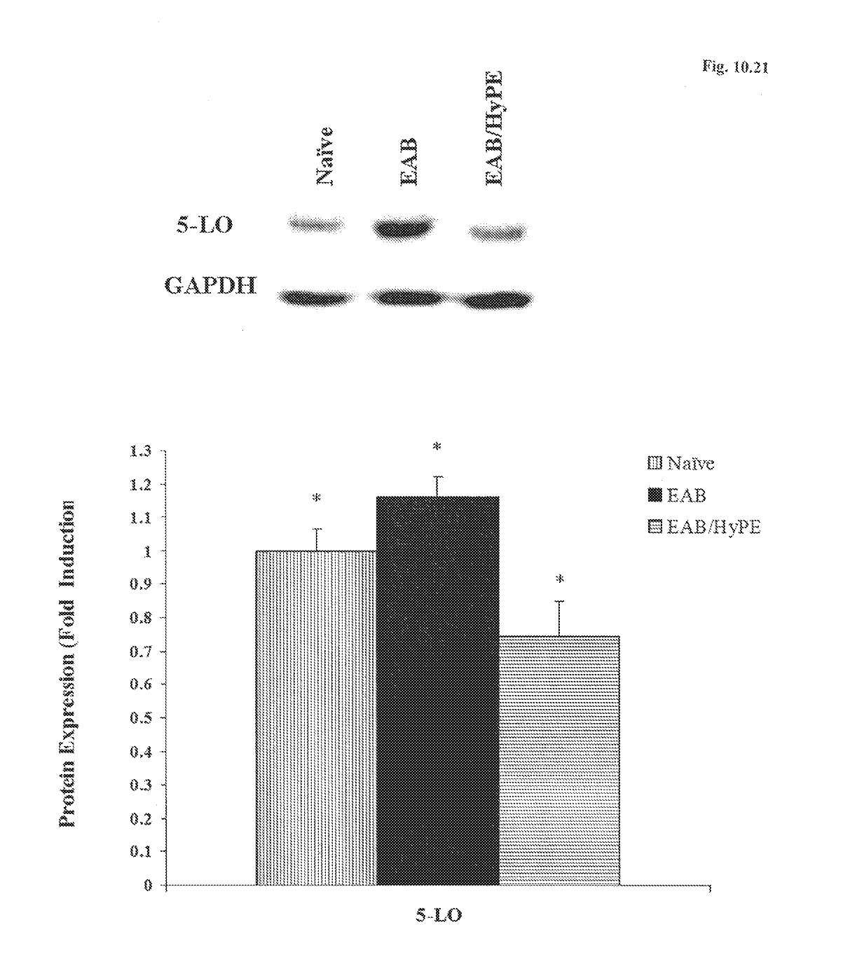

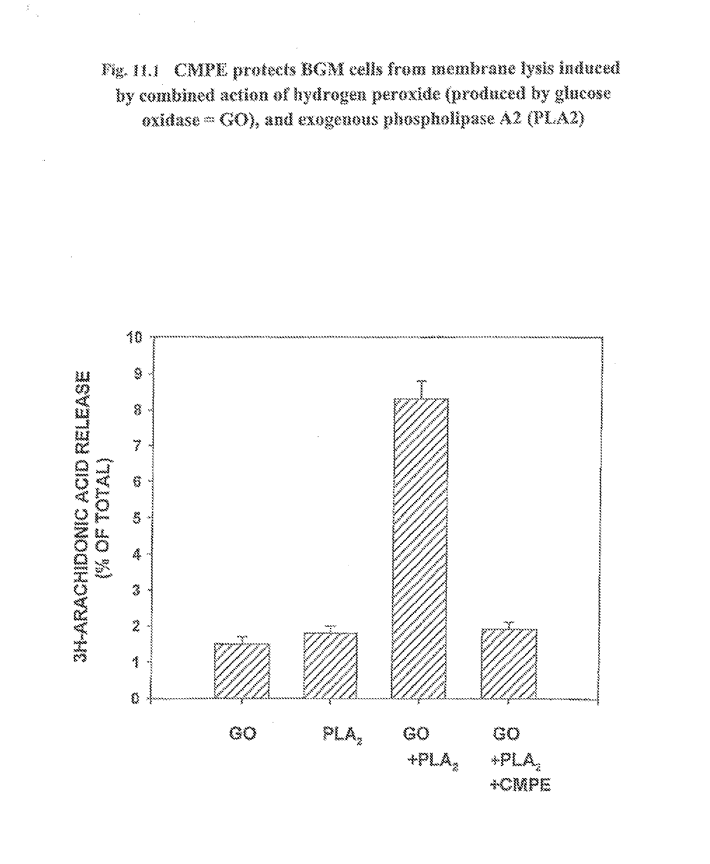

Experiment 3.1: Isolated rat tracheal rings (in a linear array) were bathed in Krebs-Hanselet buffer (pH=7.4), and linked to a tension transducer. ET-1 was added to a final concentration as indicated, and the tracheal ring contraction was determined by the change in the force applied to the tension transducer (FIG. 10.1A). Subsequently, the highest ET concentration was used in testing the Lipid-conjugates to inhibit the smooth muscle contraction. In this experiment (FIG. 10.1B), rat trachea rings were incubated with the Lipid-conjugate HyPE at the indicated concentration for 1 hr. ET-1 was then added to a final concentration of 1 μM and the ring contraction was determined as in Experiment 3.1A. Each datum is mean±S.D, of four separate experiments (4 rats).

Experiment 3.2: Rat trachea rings were incubated with 3 μM HYPE or hyaluronic acid (HA) alone, for 1 hr. ET-1 was then added to a final concentration of 1 μM (empty bars) or 10 μM (full bars) and the tracheal ring contraction was determined as in Experiment 3.1 (FIG. 10.2).

Experiment 3.3: The same as Experiment 1.2, but the tracheal ring contraction was induced by 10 μM Acetyl Choline (AcCh), as shown in FIG. 10.3.

Experiment 3.4: Guinea pig tracheal rings (in a linear array), immersed in a ringer bath, were connected to an apparatus measuring the length of the ring chain. CMPE or HEPPE was added to the bath 1 h prior to the stimulation of contraction by either Crotalus atrox (type II) enzyme or endothelin-1 as indicated (Table 3.1).

TABLE 3.1
Inhibition of Tracheal Ring Contraction by CMPE and HEPPE

| Stimulant | Lipid-conjugate | % Inhibition |
|---|---|---|
| Phospholipase (0.5 μ/ml) | CMPE (10 μM) | 100 ± 0.3 |
| (crotalus atrox type II) | | |
| Histamine (20 μM) | CMPE (10 μM) | 69 ± 0.1 |
| Histamine (20 μM) | HEPPE (15 μM) | 56 ± 0.05 |
| Endothelin-1 (100 nM) | CMPE (10 μM) | 92 ± 1.1 |

Experiment 3.5: Guinea pig tracheal rings were incubated with or without CMPE for 30 minutes prior to stimulation. The medium was collected after 30 minutes and PGE₂ and TXB₂ were determined by radioimmunoassay (Table 3.2). (n.d.=below limit of detection.)

TABLE 3.2
Inhibition of Tracheal Tissue
PGE₂ and TBX₂ Production by CMPE

| | | PGE₂ | TXB₂ |
|---|---|---|---|
| Stimulant | CMPE | (ng/ml) | (ng/ml) |
| Hitsamine (40 μM) | — | 5.1 | 5.6 |
| Histamine (40 μM) | 10 μM | n.d. | 1.75 |

Experiments 3.6-3.8 demonstrate the ability of Lipid-conjugates to exert their pharmacological effect in live animals. The following procedures were applied in these experiments:

We also investigated the involvement of PLA₂s and eicosanoids in the pathophysiology of asthma in a rat model of ovalbumin (OVA)-induced experimental allergic bronchitis (EAB), as reflected by broncho-constriction, airway remodeling, the levels of the broncho-dilator PGE₂ and the broncho-constrictor Cys-LTs in bronchoalveolar lavage (BAL), as well as of TNFα secretion by lung macrophages. We found that these indices were all up-regulated upon induction of EAB except for PGE₂ which was markedly reduced. Concomitantly, sPLA₂ expression in lung tissue was enhanced, while cPLA₂ expression was markedly decreases. All the bronchitis-associated parameters were reversed upon amelioration of the disease by treatment with a sPLA₂ inhibitor, resulting in elevation of cPLA₂ and PGE₂ along with suppression of sPLA₂ and Cys-LTs.

Inbred Brown Norway male rats (4 weeks old) obtained from Harlan, USA, were used in this study. The Hebrew University Animal Welfare Committee approved all protocols.

Induction of asthma: Asthma was induced in rats by sensitization with ovalbumin (OVA, Sigma-Rehovot, Israel) according to a previously described protocol (33): On day 0 rats received a single subcutaneous injection of 1 mg OVA+aluminum-hydroxide (200 mg/ml in 0.9% NaCl) (Sigma-Rehovot, Israel) and an intraperitoneal injection of 1 ml containing 6×109 heat-killed Bordetella Pertussis bacteria (Pasteur Marieux, France). Repeated bronchial allergen challenge was performed from day 14 every other day for 1 month by inhalation of OVA (1 mg/ml in 0.9% Normal Saline) for 5 minutes each time in a 20 L box connected to an ultrasonic nebulizer (LS 230 System Villeneuve Sur Lot, France).

Treatments:

Rats were divided into 4 treatment groups: 1. No sensitization and no treatment, used as Naïve control. 2. Sensitization+challenge with OVA and no treatment, used as positive control. 3. Sensitization+challenge with OVA and treatment with Lipid-conjugate (HyPE), either by sub-cutaneous (SC) injection or inhalation, before every challenge (HyPE). 4 (in part of the experiments)−sensitization+challenge with OVA and treatment with SC injection of dexamethasone 300 μg before each challenge (OVA/Dx). The OVA/OVA group received 1 ml saline before each challenge.

Two modes of treatments with HyPE were employed: 1. The rats received SC injection of 1 ml saline containing 15 mg HyPE (to obtain about 1 mg/ml body fluid=20 μM). 2. The rats, placed unrestrained in a 20 litre box connected to an ultrasonic nebulizer, inhaled HyPE as follows: 5 ml of 1 mg/ml HyPE was aerosolized into the 20 L cage, thus diluting the HyPE to 0.25 μg/ml aerosol. The rat respiratory rate was 120 breath/min, with a tidal volume of about 1 ml, thus reaching ventilation of 120 ml/minute. If all the inhaled HyPE was absorbed, in 5 min (inhaling 600 ml), the maximal HyPE absorbed was 150 μg.

In mode 1, all groups (5 rats in each) were treated and challenged as described above on day 14, 16, 18 and 20, and pulmonary function (Penh) was assessed on day 20 before and 5 min after challenge (EAR).

In mode 2, each group (10 rats in each) were treated and challenged from day 14, every other day, until day 45. Pulmonary function (Penh) was assessed on day 20 before and 5 min and 8 h after challenge, corresponding to early and late asthmatic reaction (EAR and LAR, respectively).

Assessment of Broncho-Constriction:

Unrestrained conscious rats were placed in a whole-body plethysmograph (Buxco Electronics Inc., Troy, N.Y., USA) connected to a pneumotach (EMKA Technologies, Type 0000) at one end, and to a 10 ml bottle at the other end. The pneumotach was connected to a preamplifier (model MAX2270, Buxco Electronics). Analogue signals from the amplifier were converted to a digital signal by an AD card (LPM-16 National Instruments, Austin, Tex., USA). Broncho-constriction measures were expressed as the enhanced pause (Penh). Penh=(PEF/PIF)*((Te−Tr)/Tr), where PEF=Peak Expiratory Flow, PIF=Peak Inspiratory Flow, Te=Expiratory Time, Tr=Relaxation time=time of the pressure decay to 36% of total box pressure during expiration.

Broncho-Alveolar Lavage (BAL):

On day 45 the rats were sacrificed by bleeding through the abdominal aorta under anaesthesia with intra-peritoneal injection of sodium pentobarbital (100 mg/kg). The rats were tracheotomized and incannulated through the trachea. Bronco-alveolar lavage (BAL) was collected by repeated washing of the lungs with 5 ml saline to a total of 50 ml.

Assessment of Airway Pathology:

Subsequent to collection of BAL, lungs were removed and inflated with 4% buffered formaldehyde under pressure of 20 cm H2O. The lungs were sliced longitudinally and embedded in paraffin. Histological sections 3 μm thick were cut and stained with hematoxylin and eosin for assessments of interstitial and peri-bronchial inflammation and airway smooth muscle thickening. Other slides were stained with Tri-chrome for assessment of sub-epithelial fibrosis (basal membrane) and with PAS for epithelial cell mucus metaplasia.

Histological morphometry of airway structural changes was performed using a computer program “ImageJ” (NIH Bethesda USA) on 3 randomly selected slides from each mouse. Quantification of peribronchial cellular infiltrate in airway tissue was achieved through counting the numbers of these cells in the 50-μm region beneath the epithelium of the airway in hematoxylin and eosin stained sections. Cells were expressed as number per millimeter of airway basal lamina length, which was measured by tracing the basal lamina in calibrated digital images. Morphometric analysis of ASM and the basal membrane mass as indices of their thickening were performed as previously described. Briefly, measurements of the airway were obtained by tracing the digitalized images of interest. The outlines of the airway structures were subsequently measured. All airways were evaluated for the following morphometric dimensions: length of the airway basement membrane of the epithelium (Lbm) and area of the ASM in the eosin hematoxylin stained slides and the blue stain of the basal membrane of the Tri-chrome stained slides. ASM cells or the basal membrane thickening were normalized to the square of the Lbm (in μm2) to correct for differences in airway size. Only large (>2,000 μm Lbm) and medium size airways (1,000-2,000 μm Lbm) were selected as it was shown that the most significant pathological changes occur in these airways.

Protein Expression of sPLA2 in Lung Tissue:

Proteins were identified in homogenized lung tissue (100 g protein) using standard Western blot. A specific polyclonal antibody against Anti-sPLA2 antibody (Santa Cruz) diluted 1:500 (viv) in TBST buffer+0.1% BSA. The immune reaction was detected by enhanced chemiluminescence (ECL).

Cysteinyl Leukotriene (CysLT):

CysLT levels were measured in BAL using a kit for direct enzyme immunoassay (EIA), according to manufacturer's instructions (Amersham Pharmacia Biotech U.K). The specificity of the kit was 100% for LTC4, 100% for LTD4, and 70% for LTE4. Result range was between 0 to 48 pg.

Cell culture—Cells were isolated from the BAL were suspended in DMEM medium supplemented with 10% fetal calf serum (FCS) and plated in a 96-well plate at 106 cells/well. The cells were incubated for 2 hours in 37° C., then non-adherent cells were removed by washing with PBS. The adherent cells were re-suspended in DMEM supplemented with 10% FCS at 106 cells/well and incubated for 48 hours. The culture medium was then collected and assayed for determination of biochemical markers.

Nitric Oxide (NO) production—NO production by the BAL cultured macrophages was determined by measuring their level in the culture medium using the photometric method of Griess et al.

TNFα production: TNFα production by the BAL cultured macrophages was determined in the culture medium using radio-immunoassay (RIA) kits [Amersham-Pharamcia, UK).

Statistical Analysis:

All data are expressed as mean±SEM. One way ANOVA was used to compare treatment groups. Pair-wise comparisons were performed by the Tukey-Kramer HSD test (p=0.05). Where necessary, data were log transformed before analysis to stabilized variances. In all analyses P<0.05 was considered statistically significant.

Statistics:

Statistical analysis was performed using statistical software (GB-STAT, Dynamic Microsystem Silver Spring Md., USA. Analyzis of variance (ANOVA) was used to assess difference of the results of the treatment groups. A Tukey test was used to compare between each one of the treatment groups. A value of p<0.05 was considered as a significant difference.

Experiment 3.6—demonstrates that SC-administration of Lipid conjugates considerably ameliorate OVA-induced broncho-constriction (FIG. 10.4, bronchoconstriction was induced in OVA-sensitized rats by inhalation of OVA, and expressed by the difference in Penh measured before and 5 min after allergen challenge. Each datum is Mean±SEM for 10 rats. Statistical significance: a—P<0.01; b, c—P<0.05), reduced the expression of secretory phospholiapse (FIG. 10.5, the figure depicts Western blot and corresponding densitometry of sPLA₂ in lung homogenates of rats with OVA-induced asthma, treated as indicated. In panel B, for each enzyme the density values were normalized to corresponding Naïve), and prevented the production of the broncho-constricting lipid mediators cysteinyl leukotrienes (FIG. 10.6, broncho-alveolar lavage (BAL) was collected upon sacrifice and CysLT levels were determined by EIA, as described in Methods. Each datum is Mean±SEM for 10 rats. Statistical significance: a, b−P<0.01. No significant difference between HyPE treated and the Naive rats).

Experiment 3.7 (aerosolic administration of HyPE) demonstrates that treatment of the asthmatic rats by inhalation of the Lipid-conjugate, reduces protects the rats from sensitization by OVA, as it markedly reduced OVA-induced broccho-constriction in both the early and late asthmatic reaction (FIG. 10.7, bronchoconstriction, expressed as the percent change of Penh was induced in OVA-sensitized rats by inhalation of OVA, and measured before allergen challenge, min and 8 h after allergen challenge. Each datum is Mean±SEM for 10 rats. Two experiments were performed for EAR. 5 rats were included in each group in the first experiment. The same experiment was repeated with 10 rats in each group, which were further used for determination of LAR. Combined statistical test for EAR yielded p<0.01 between Asthmatic and HyPE-treated; no significant difference between the HyPE-treated and the Naive or Dx-treated groups. For LAR, p<0.01 between Asthmatic and HyPE-treated; no significant difference between the HyPE-treated and the Naive or Dx-treated groups), inhibited the production of CysLT, potent brocnho-constricting lipid mediator (FIG. 10.8, broncho-alveolar lavage (BAL) was collected upon sacrifice and CysLT levels were determined by EA. Each datum is Mean±SEM for 10 rats. P<0.01 between asthmatic and HyPE-treated rats. No significant difference between HyPE treated and the Naive rats), and of nitric oxide (NO), a characteristic constrictor of smooth muscle cells (FIG. 10.9, macrophages, collected from the BAL of the different groups, were cultured without further treatment with HyPE or Dx, and NO production was determined as described in Methods. Each datum is Mean±SEM for 10 rats. NO level was reduced compared to asthmatic and naïve rats by both HyPE, p<0.001 and p<0.001 respectively and by Dx p<0.001 and p<0.001, respectively.) These treatments also prevented the asthma-associated inflammation, as expressed by prevention of inflammatory cell infiltration and airway remodeling (FIG. 10.10, rats were subjected to OVA inhalation every other day for 30 days. For treatment with HyPE, the rats inhaled HyPE aerosol for 5 min before every allergen inhalation. The rats were sacrificed on Day 45. A—Staining with hematoxylin eosin for detection of inflammatory cell infiltration and changes in smooth muscle cell (ASM) thickness. B—Staining of connective tissue (collagen) with Mason-Trichrom, for detection of changes in basal membrane thickness. C—Staining with Periodic Acid Schiff (PAS) for detection of mucus metaplasia of respiratory epithelial cells. 1, 2, 3 and 4 depict tissues of Naive, Asthmatic, HyPE-treated and Dx-treated rats, respectively, and FIG. 10.11), and production of TNF-alfa by lung macrophages (FIG. 10.12, macrophages, collected from the BAL of the different groups, were cultured without further treatment with HyPE or Dx, and NO production was determined as described in Methods. Each datum is Mean±SEM for 10 rats. p<0.001 between asthmatic and HyPE-treated rats. No significant difference between HyPE-treated, Naive and Dx-treated rats).

Experiment 3.8, in which HyPE was given as aerosol to only before challenge to rats that had been sensitized by OVA (HyPE was not given during sensitization as in Experiment 3.7), demonstrates that inhalation of Lipid conjugates is effective in preventing allergen-induced broncho-condtriction in already asthmatic subjects when inhaled before allergen (OVA) challenge (FIG. 10.13, OVA-sensitized asthmatic rats inhaled nebulized HyPE (1 mg/ml) for 5 minutes, or nebulized normal saline. 30 minutes later all were challenged by inhalation of OVA (1 mg/ml) for 5 minutes. Penh was measured before the treatments (baseline), and 5 minutes after each inhalation. Each datum is mean±SEM for 5 rats. *,**, P<0.05), and reverse broncho-constriction (induce broncho-dilation) when inhaled after allergen challenge. FIG. 10.14: OVA-sensitized asthmatic rats challenged by inhalation of OVA (1 mg/ml) for 5 minutes. 30 minutes later they were treated by inhalation of nebulized HyPE inhalation (1 mg/ml) or nebulized or with normal saline for 5 minutes. Penh was measured before challenge (baseline), and after challenge and treatment. Each datum is mean±SEM for 5 rats. *, P<0.05.

We also examined the role of PLA2s in OVA-induced EAB in mice using the same methodology applied in our previous study with rats. It was found that, similar to rats, in mice OVA-induced EAB was associated with induction of sPLA₂ expression (specifically X). However, unlike EAB in rats, where the disease development is associated with suppression of cPLA₂ expression and PGE₂ production, both were elevated in mice subjected to OVA-induced EAB. Yet, in both mice and rats, the disease was markedly ameliorated by treatment with a cell-impermeable sPLA2 inhibitor.

Induction of Experimental Allergic Bronchitis (EAB) in Mice:

EAB was induced in BALB/c female mice by sensitization with three weekly intra-peritoneal (IP) injections of 0.3 ml solution containing of 0.3 mg/ml OVA as the allergen and 6.7 mg/ml aluminum hydroxide [Al(OH)₃] as the adjuvant. Challenge was applied by intranasal (IN) administration of 50 μL of OVA (2 mg/ml in PBS), three times a week for four weeks.

The EAB development was assessed by two common tests: Pulmonary function, assessed by air response to allergen or methacholine, using two non-invasive methods: (a) Enhanced pause by whole body plesthimography and (b) airway resistance.

Enhanced pause (Penh): Unrestrained conscious mice were placed in a whole body plethysmograph (Buxco Electronics, Troy, N.Y., USA) to measure flow derived pulmonary function (Penh) as previously described.

FIG. 10.15 shows that methacholine challenge of mice with OVA-induced EAB exerted airway resistance to air flow in a dose-dependent manner, and this was prevented by treatment with the sPLA₂ inhibitor HyPE. In accord with these findings, FIG. 10.16 shows that mRNA expression of arginase I and chitinase was enhanced 30-fold and 15-fold, respectively, in the lungs of mice with EAB compared to naïve mice. Together FIGS. 10.15 & 10.16 show that, as previously found in rats, treatment with the sPLA₂ inhibitor suppressed physiological and molecular manifestations of bronchitis, suggesting that it prevented the development of the inflammatory state.

Airway resistance was measured using the occlusion technique (Rocclud)—applied to to non-sedated mice breathing through a nose-mask while their mouth closed. The mask was connected to a pneumotach (flow meter) with a mouth pressure port, attached to 2 differential pressure transducers, connected to preamplifiers (Hans Rudolph, Shawnee, Kans., USA). The analog signals were converted to digital and collected by a data acquisition program (LabView National Instruments, Austin, Tex., USA). Peak pressure was measured while the mouse was breathing against an occluded pneumotach for 3-5 breaths. The pressures generated at the beginning and at the end of the occlusion (inspiratory and expiratory, respectively) were divided by the respective adjacent peak flow immediately before and after the occlusion. Resistance (Rocclud) was calculated as peak pressure divided by the adjacent peak flow.

Airway reactivity was assessed before challenge (baseline), and 5 minutes after challenge, by intranasal instillation (IN) of either OVA (100 μg in 50 μL PBS) or increasing methacholine dose (0, 40, 80, 320, 640, and 1280 μg in 20 μL PBS). Airway resistance is expressed as the percent change compared to baseline.

FIG. 10.17, shows that mice with OVA-induced EAB respond to OVA challenge with significant increase in airway resistance, as expressed both by Penh (FIG. 10.17A) and percent change of resistance (FIG. 10.17B). Similarly, FIG. 10.18 shows that the EAB induction in mice was associated with peribronchial infiltration of inflammatory cells, as demonstrated by the lung histology micrograph (FIG. 10.18A) and the respective morphometric measurement (FIG. 10.18B).

FIGS. 10.17 & 10.18 also show that the pre-treatment with the PLA₂ inhibitor completely prevented the disease development, as reflected by the suppression of bronco-constriction (FIGS. 10.17A & 10.17B) and inflammatory cell infiltration (FIGS. 10.18A & 10.18B), reverting these markers to their levels in naïve mice.

Gene expression of Arginase-1 and mammalian acidic chitinase in lung tissue, known to be enhanced in asthma. Arginase-1 is involved in the metabolism of L-arginine and subsequent inhibition of production of nitric oxide (NO), typical of type-2 responses. Although chitin does not exist in mammals, chitinases and chitinase-like proteins have recently been observed in mice and human subjects. The prototypic acidic mammalian chitinase is induced during T_{H}2 inflammation through an IL-13-dependent mechanism, and plays an important role in the pathogenesis of T_{H}2 inflammation and IL-13 effector pathway activation.

Broncho-Alveolar Lavage (BAL)

BAL was collected by incanulation of the trachea and lungs were washed three times with 2 ml PBS. The BAL fluid was centrifuged to remove the cells and kept at −80° C.

PLA₂ mRNA Expression in Lungs Tissues

Lung tissues were excised and frozen immediately at −80° C. in eppendorf tubes containing RNAlater (Ambion by life technologies, Austin, Tex.). Total RNA was isolated using the SV Total RNA isolation kit including a DNase I treatment (Promega corporation, Madison, Wis.) to remove any possible contamination of gemonic DNA. RNA quality was assessed using 1% agarose gel electrophoresis. MuLV reverse transcriptase with oligonucleotides and random primers (Applied Biosystems) were used for one round of poly chain reaction (PCR), in order to prepare cDNA. All sets of primers were designed using the Primer Express program from Applied Biosystem and Blastn (Pub med), taking into consideration the amplicon length, thermal cycles in the Real-Time PCR and CG nucleotides content of the primers. The abundance of the target mRNA was calculated relative to the expression of the 18S ribosomal RNA that served as a reference gene (endogenous gene), while the naive group was used as calibrating factor. The thermal cycles were set at 94° C. for 5 min, followed by different number of cycles depending on the gene (40-50 cycles), each comprising a denaturation step at 94° C. for 15 sec, an annealing step at 60° C. for 30 sec, an extension step at 72° C. for 20 sec, and an additional extension step after all cycles at 72° C. for 10 min. The amplification of the appropriate product was verified in all reactions by analyzing the dissociation curves that were obtained after PCR as followed: ramp from 72° C. to 99° C., rising 1° C. each step, for 45 sec for the first step and 5 sec for each step afterward. In this test we focused on PLA₂s that have been reported to be implicated involved in asthma pathophysiology in mice, specifically sPLA₂gV, sPLA₂gX and cPLA₂γ (PLA₂gIVC).

As shown in FIG. 10.19, while sPLA₂gV, sPLA₂gIB and sPLA₂gIII were not affected by the disease induction, the expression of both sPLA₂gX and cPLA₂γ was markedly increased, and were suppressed by treatment with the sPLA₂ inhibitor. The elevated expression of sPLA₂ is in agreement with our findings in the rat model, as well as with other studies in mice. However, the elevated cPLA₂ expression, while in agreement with others' studies, is in contrast to our findings with the rat model, where cPLA₂ expression was suppressed in the disease state and resumed upon treatment with the sPLA₂ inhibitor.

Eicosanoids Level in BAL

Cysteinyl-Leukotrienes (Cys-LT's), prostaglandins E₂ and D₂ (PGE₂ and PGD₂), and thromboxane B₂ (TXB2) were determined in BAL using a kit for competitive enzymes immunoassay (ELISA), according to the manufacturer's instructions (Cayman Chemical, Michigan). The specificity of the Cys-LT's kit, was 100% for LTC₄ and LTD₄, and 79% for LTE₄. The result range was between 34 and 103 pg/ml. The specificity of the PGE₂ kit was 100% for PGE₂, PGE₂-Ethanolamide and PGE₂-1-glyceryl ester. The result range was between 15 and 50 pgiml. The specificity of the PGD₂ kit was 100% for PGD₂, and 92.4% and 21.6% for its metabolites PGF₂α and PGJ₂, respectively. The result range was between 55 and 240 pg/ml.

FIG. 10.20 shows that in the mouse model disease induction is associated with enhanced production of both types of eicosanoids—the broncho-dilating PGE₂ and the broncho-constricting CysLTs-PGD₂ TXB₂, as well as elevated expression of both cPLA₂ and sPLA₂. Yet, similar to the EAB in rats, the treatment with the extracellular sPLA₂ inhibitor reduced the expression of both types of PLA₂s and eicosanoid production, concomitantly with amelioration of the disease.

Western Blotting Analysis of 5-Lipoxigenase (5-LO) and 15-Lipoxigenase (15-LO) in Lung Tissue

Lung tissues were homogenized with lysis buffer [1% NP40, 0.50% sodium deoxycholate, 0.1% sodium dodecyl sulfate (SDS), 150 mM NaCl, 10 mM buffered phosphate pH 7.2, 2 mM EDTA, 50 mM NaF, 0.2 mM orthovanadate and protease inhibitor cocktail], and incubated on ice for 15 min. The lysates were then centrifuged at 20 000 g for 15 min and pellets were discarded. Protein concentration of each sample was determined using Bradford Reagent (Sigma). Samples containing 20 mg protein were boiled in 1 SDS sample buffer, separated by SDS-10% polyacrylamide gel electrophoresis (PAGE) and blotted onto polyvinylidene difluoride (PVDF) membranes (Millipore, Bedford, Mass., USA). The membranes were incubated in 5% fat-free milk in TBST (10 mM Tris-HCl pH 7.4, 150 mM NaCl, 0.1% Tween 20) for 1 h, and then with rabbit anti-mouse 5- or 15-LO antibodies in 5% bovine serum albumin (BSA) in TBST for 18 h at 4° C. The membranes were washed three times with TBST for 5 min each before and after incubation with an appropriate secondary antibodyn (1 h, 20-25° C.) coupled to horseradish peroxidase-conjugated goat anti-rabbit antibody, and visualized by chemiluminescence according to the manufacturer's instructions (West Pico, Pierce, Rockford, Ill., USA).

FIG. 10.21 shows that the EAB induction was associated with elevation in 5-LO protein expression, which was suppressed by the treatment with the sPLA₂ inhibitor, whereas 15-LO expression was not affected by the disease or its treatment.

Treatment with Cell-Impermeable sPLA₂ Inhibitor

As in the previous study of EAB in rats, we have tested here the effect of the cell-impermeable sPLA₂ inhibitor, composed of PLA₂-inhibiting lipid (specifically derivatized phosphatidyl ethanolamine) conjugated to truncated hyaluronic acid (HyPE), which prevents the inhibitor internalization, thereby designed to confine its inhibitory action to the cell membrane. This inhibitor has been shown to suppress the action of exogenous sPLA₂s and diverse related inflammatory conditions in numerous to studies. As noted above, the first stage of IP injection of OVA (3 weekly IP injections), was followed by challenge with IN OVA administration every other day for 4 weeks. At this stage, HyPE was administered IN an hour before each challenge with OVA (50 μl of 4 mg/ml solution at the first two challenges, followed by 40 μl of 1 mg/ml, until one day before sacrificing).

Statistical Analysis

For all assays statistical significance was determined using one-way analysis of variance (ANOVA), followed by Tukey multiple comparison. Conventionally, a P value of less than 0.05 was considered significant.

These experiments demonstrate that the Lipid-conjugates may be used for the treatment of obstructive respiratory disease, asthma and bronchitis, alleviating airway narrowing by a plurality of mechanisms, including inhibition of contraction and reduction of airway obstructing infiltrates.

### Example 4

### Anti-Oxidant Therapy

The Lipid-conjugates are effective therapy for preventing oxidative damage. This is demonstrated in Experiments 4.1-4.3. The noxious effect of peroxide free radicals on living tissue is known as oxidative damage. When cell membranes are the targets for this damaging process, membrane dysfunction and instability result. Oxidative damage to blood proteins, particularly blood lipid proteins, results in their over-accumulation in cells lining the vasculature, thus contributing to atherogenesis. In fact, oxidative cell damage is a major mechanism attributed to the process of aging or senescence.

Oxidative damage to proteins or cell membranes is commonly assessed by exposing these tissues to hydrogen peroxide produced by the enzyme glucose oxidase (GO), in the absence or presence of additional membrane destabilizing agents, such as PLA₂, or by exposure to divalent cations, such as copper.

Experiments 4.1-4.3 demonstrate the ability of Lipid-conjugates to preserve cells from oxidative damage, as judged by the cells' retention of both arachidonic acid and of low molecular weight intracellular substances.

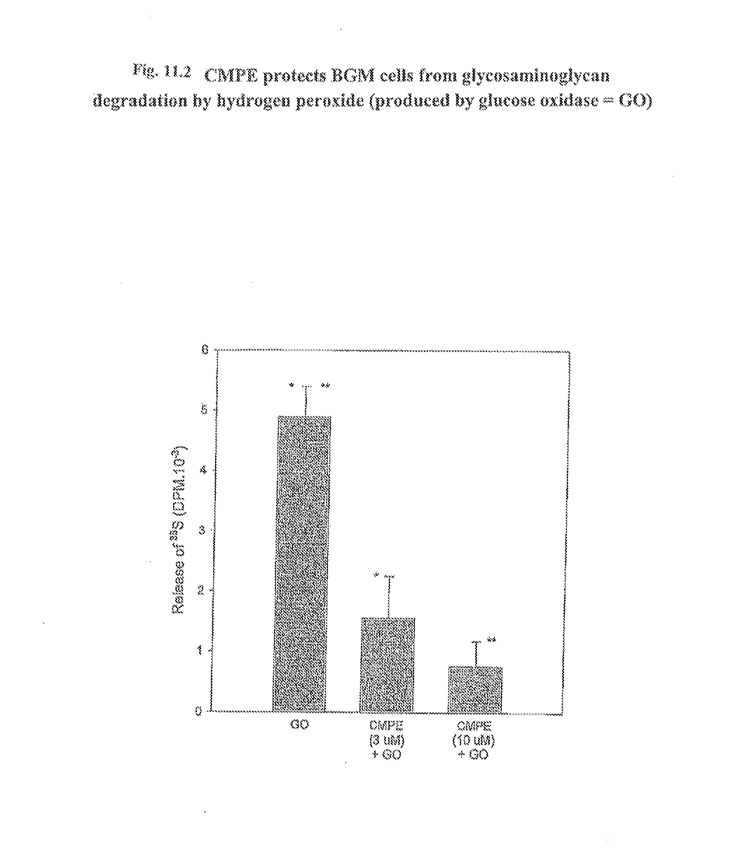

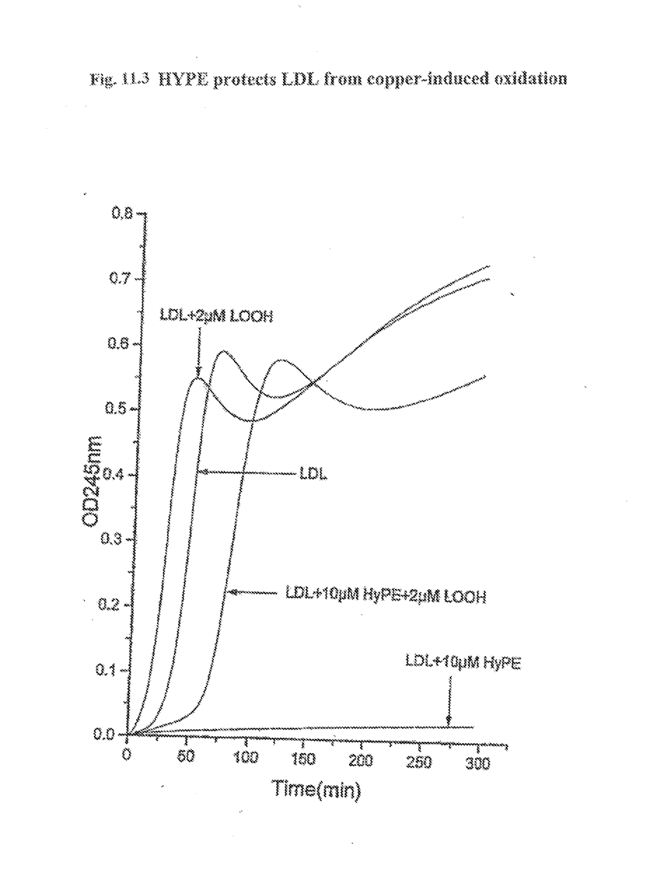

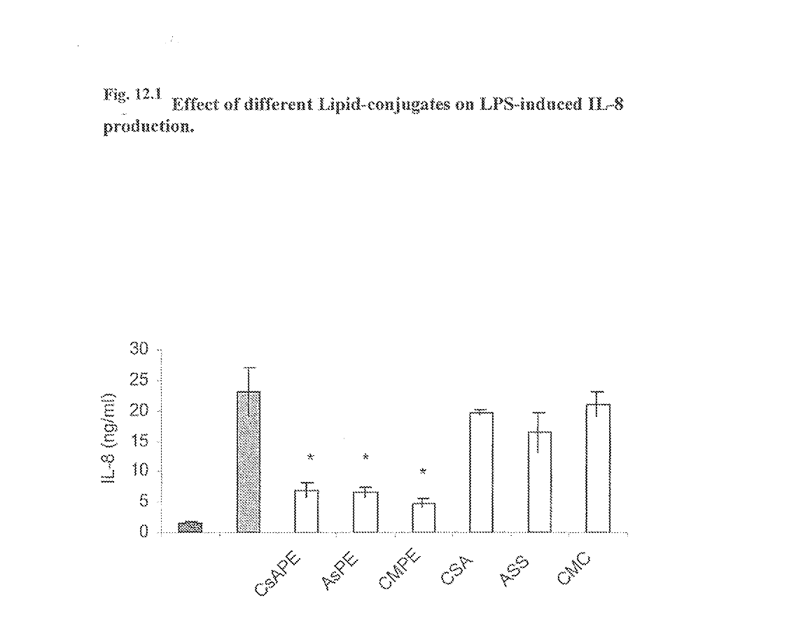

Experiment 4.1: Confluent BGM (green monkey kidney epithelial cells) were labeled with ³H-arachidonic acid. The cells were treated with CMPE for 30 min prior to treatment with GO and PLA₂ (0.5 u/ml) (FIG. 11.1).

Experiment 4.2: BGM cells were labeled with ³⁵SO₄ overnight. The cells were washed with DMEM (containing 10 mg/ml BSA) 4 times with PBS. The cells were then incubated in DMEM supplemented with GO (an H₂O₂ generation) for 90, and the culture medium was collected and counted for ³⁵S radioactivity. For treatment with CMPE cells were incubated with CMPE, at the indicated concentration for 30 min prior to introduction of GO. Each datum is MEAN+SEM for 5 replications. *p<0.005; **p<0.001 (FIG. 11.2).

Experiment 4.3: For demonstrating the ability of Lipid-conjugates to inhibit the oxidation of blood lipoprotein. LDL (0.1 μM) was incubated in the absence and presence of various concentrations of HYPE or HA at 37° C. At time zero 5 M CuCl₂ was added to the dispersions and the mixtures were continuously monitored for oxidation products at 245 nm (FIG. 11.3). The absorbance at 245 (OD units) is depicted as a function of time (Schnitzer et al., Free Radical Biol Med 24; 1294-1303, 1998).

These experiments demonstrate that administration of Lipid-conjugates is effective therapy in the prevention of tissue damage induced by oxidative stress (associated with free radical and hydrogen peroxide production) by a plurality of mechanisms, including inhibiting the oxidation of lipoprotein, as well as their uptake, inhibiting arachidonic acid release, and preserving the integrity of cell membranes (inhibiting GAG degradation), including red blood cell membranes.

### Example 5

### Lung Injury/Acute Respiratory Distress Syndrome (ARDS)

In acute respiratory distress syndrome (ARDS), which is usually induced by bacterial endotoxins (LPS, LTA), a high production of injurious mediators, particularly neutrophil-attracting chemokines, and cytokines, are produced by the lung microvascular endothelial cells (LMVEC). To demonstrate the ability of the Lipid-conjugates to control the production of these injurious agents, LMVEC were treated with LPS (gram-positive bacterial endotoxin) and LTA (gram-negative to bacterial endotoxin), in the absence and presence of Lipid-conjugates, and tested for the subsequent production of cytokines and adhesion molecules.

To this end, human lung microvascular endothelial cells (LMVEC) were purchased from CellSystems, Remagen, Germany at passage 4. The cells were seeded in a density of 5000 cells^{-cm2} in T25 flasks and maintained according to the manufacturer's specification in EGM-MV. Characterization of the LMVEC was performed on the basis of a positive staining for uptake of acetylated LDL, Factor VIII related antigen and PECAM (CD31) expression as well as negative staining for alpha smooth muscle actin. In each experiment the viability of LPS- and LTA-stimulated or HyPE-treated LMVEC was tested by trypan blue exclusion. The production and mRNA expression of cytokines and adhesion molecules were determined as described in U.S. application Ser. No. 10/989,606 filed 17 Nov. 2004, which is incorporated herein by reference in its entirety.

The production of the chemokines ENA-78, Gro-α and IL-8, secreted into the culture medium of stimulated LMVEC, was measured by ELISAs according to the manufacturer's instructions.

For RNA isolation and Polymerase Chain Reaction by RT-PCR, confluent LMVEC were stimulated with medium as control or with LPS (1 μg^{-ml}) or LTA (10 μg^{-ml}) in the presence or absence of HyPE (10 μM). Total RNA was isolated using Trizol-Reagent according to the manufacturer's instructions. Each RNA preparation was subjected to DNAse digestion to remove possible contaminations of genomic DNA. 1 μg of total RNA was reverse transcribed using SuperScript™ II Preamplification System according to the manufacturer's instructions. Amplification of 0.5 μl of cDNA was performed in a total volume of 25 μl containing 19.6 pmol of each chemokine primer, 5 mM of dNTPs, 2.5 U Taq Polymerase, 10 mM Tris HCl, 7.5 mM KCl, 1.5 mM MgCl₂. PCR reactions were initiated at 94° C. for 3 min, followed by 30 cycles of amplification, each consisting of 94° C. for 1 min, 58° C. for 1 min, 72° C. for 2 min. At the end of the amplification cycles the products were incubated for 10 min at 72° C. Control samples were constructed either by omitting cDNA synthesis or without addition of cDNA. PCR products were separated on a 1% agarose gel. Real-time PCR: 500 ng of total RNA of each sample was in addition reverse-transcribed into cDNA for Real-time PCR analysis using 1st Strand cDNA Synthesis Kit according to the manufacturer's instructions (Roche). cDNA was diluted in 201 DEPC-treated water. DNA standards were generated by PCR amplification of gene products, purification and to quantification by spectrophotometry. Real time PCR of cDNA specimens and DNA standards were performed in a total volume of 25 μl in the presence of 2 μl Light cycler-FastStart DNA Master SYBR GreenI reaction mix, 0.5 μM of gen-specific primers and 4 mM MgCl₂. Standard curves were generated for all chemokines. PCR efficiency was assessed from the slopes of the standard curves and was found to be between 90% and 100%. Concentration of chemokine cDNA was calculated by linear regression analysis of all standard curves and was corrected for an equal expression of GAPDH. At least five reproducible experiments were performed.

Adhesion molecules ICAM-1 and p-selectin were determined by fluorescence-activated cell sorter (FACS); Confluent LMVEC were stimulated with medium as control or with LPS (1 μg^{-ml}) or LTA (10 μg^{-ml}) in the presence or absence of HyPE (10 μM). Thereafter cells were harvested by T/E, extensively washed and monoclonal antibodies directed against the endothelial adhesion molecules ICAM-1 and P-selectin in dilutions of 1:20 were added for 30 min at 4° C. In addition unstimulated or stimulated cells were harvested as described and preincubated for 20 min with HyPE (10 μM) and monoclonal antibodies against TLR4. Cells were washed and incubated with an anti-mouse F(ab′)2, FITC conjugated secondary antibody. After washing cells were analyzed by FACS-scan.

Expression of NFRcB was determined by Electrophorese mobility shift assay (EMSA); Confluent LMVEC were preincubated overnight in basal medium containing 0.01% BSA. Thereafter they were stimulated or not for different time periods with LPS, IL-1 or TNF-α in the presence or absence of HyPE, and respective nuclear extracts were prepared. Oligonucleotides containing a NFkB consensus sequence (5′-AGT TGA GGG GAC TTT CCC AGG C-3′) were labeled to a specific activity>5×107 cpm^{-μg} DNA. NF-kB-binding was performed in 10 mM HEPES, (pH=7.5), 0.5 mM EDTA, 70 mM KCl, 2 mM DTT, 2% glycerol, 0.025% NP-40, 4% Ficoll, 0.1 M PMSF, 1 mg^{-ml} BSA and 0.1 μg^{-μl} poly di/dc in a total volume of 20 μl. Nuclear extracts (10 μg) were incubated for 30 minutes at room temperature in the presence of 1 ng labeled oligonucleotide. DNA-protein complexes were resolved on 5% non-denaturating polyacrylamide gels electrophoresed in low ionic strength buffer and visualized by autoradiography. Specificity of shifted bands was demonstrated by adding a cold NFkB consensus sequence or by supershift using anti-p65 antibodies.

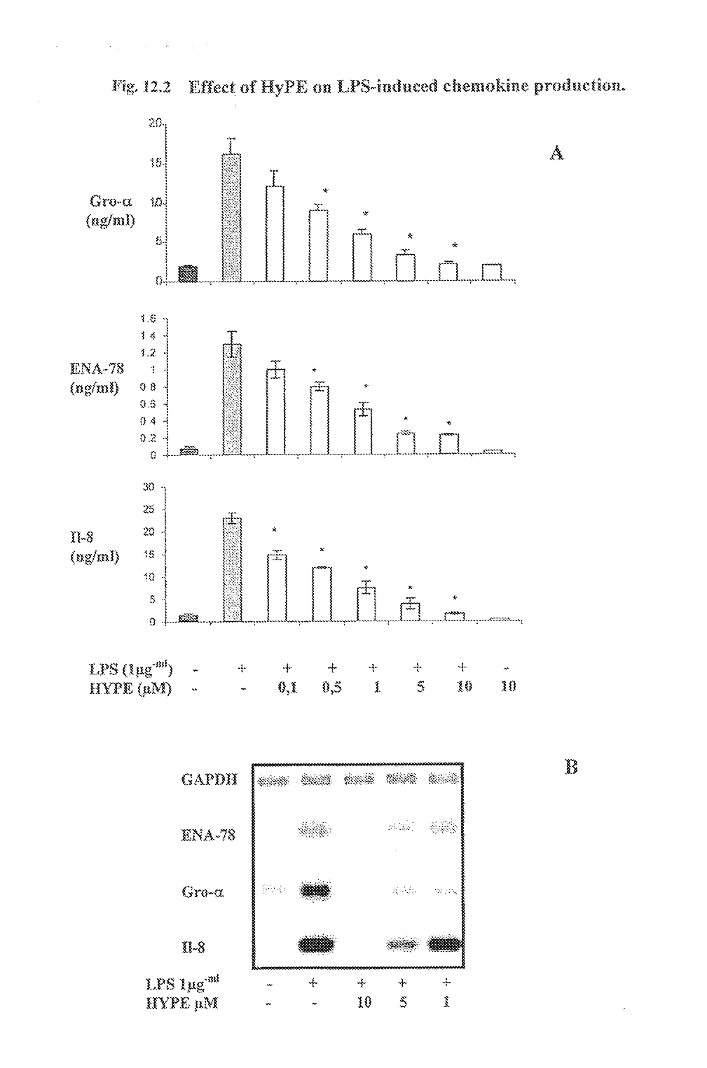

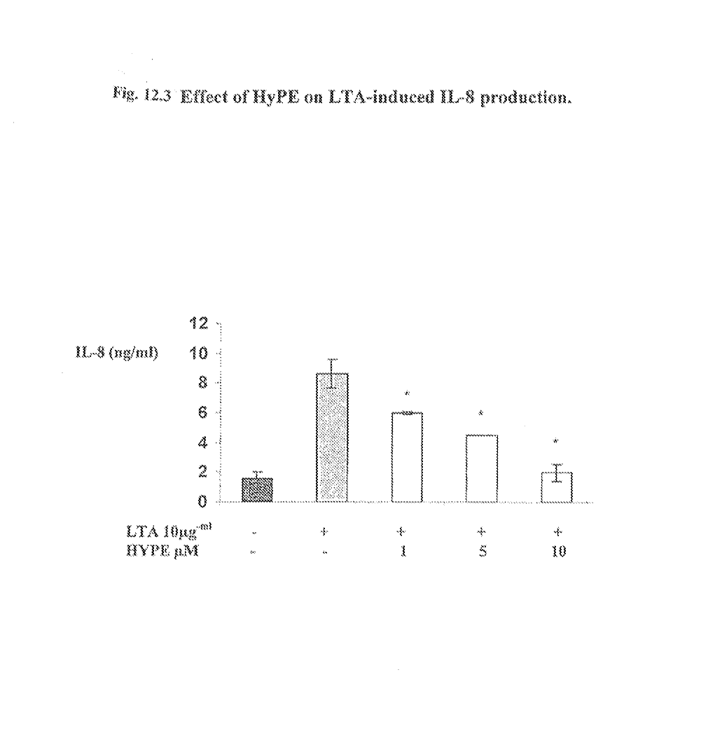

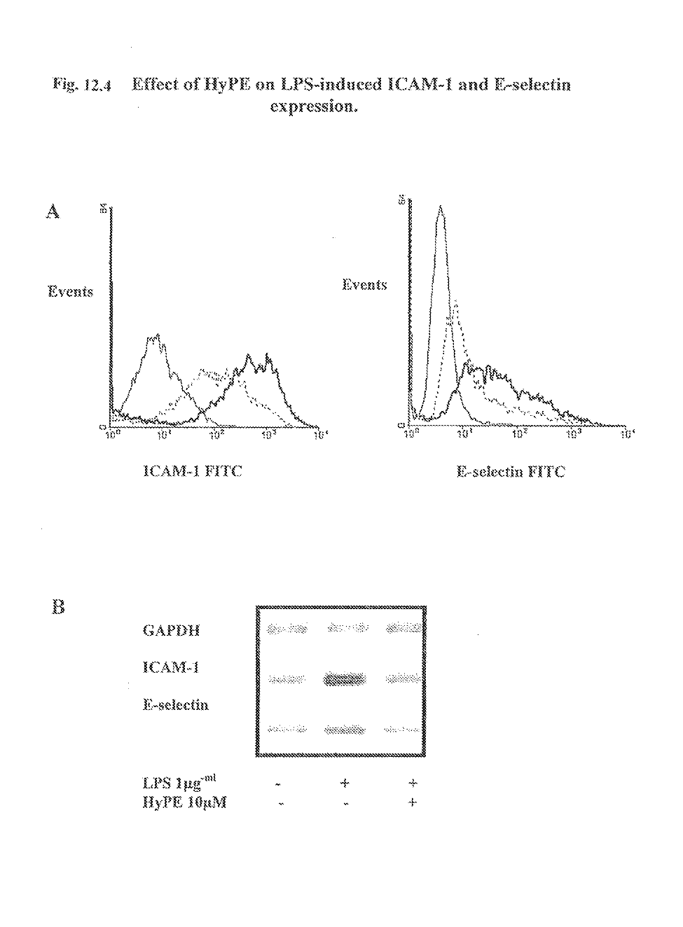

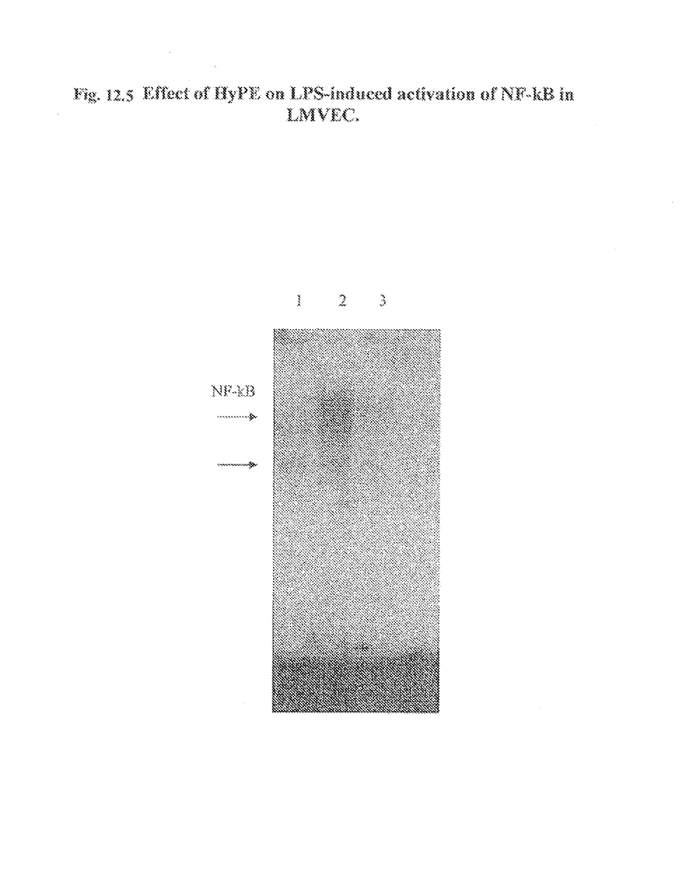

Experiment 5.1 demonstrates that the Lipid-conjugates are effective in suppressing the endotoxin-induced production and RNA expression of the chemokines IL-8, ENA-78 and Gro-α and their mRNA expression, as shown in FIGS. 12.1, 12.2 and 12.3.

Experiment 5.2 demonstrates that the Lipid-conjugates are effective in suppressing the expression of the adhesion molecules ICAM-1 and E-selectin (FIG. 12.4).

Experiment 5.3 demonstrates that Lipid-conjugates are effective in suppressing the expression of NFacB, the transcription factor that is enhanced in endotoxin-induced injurious states (FIG. 12.5).

These results further demonstrate the therapeutic capacity of the Lipid-conjugates in the treatment of ARDS and lung injuries, as well as other disease that share common mechanisms, such as peritonitis, kidney failure, organ transplantation and the like.

### Example 6

### Toxicity Tests

Experiment 6: The following compounds were tested: HyPE, CMPE, CSAPE and HepPE. The compounds were injected P at one dose of 1000, 500 or 200 mg/Kg body weight. Toxicity was evaluated after one week, by mortality, body weight, hematocrit, blood count (red and white cells), and visual examination of internal organs after sacrifice. These were compared to control, untreated mice. Each dose was applied to a group of three mice. No significant change in the above criteria was induced by treatment with these compounds, except for the HepPE, which induced hemorrhage.

The non-toxicity of the Lipid conjugates is demonstrated in Table 6.1 and Table 6.2, depicting the results obtained for HyPE in acute (6.1) and long-term (6.2) toxicity tests.

TABLE 6.1
Acute toxicity

| Dose of | | | | | |
|---|---|---|---|---|---|
| HyPE (mg/ | | | | | |
| kg body | | | RBC × | WBC × | Hemato- |
| weight) | Body weight (g) | | 10⁶ | 10³ | crit % |
| 0.0 | 21.9 ± 0.2 | 22.6 ± 0.3 | 10.7 ± 0.4 | 9.3 ± 0.3 | 45.0 ± 0.5 |
| (control) | | | | | |
| 250 | 22.1 ± 0.4 | 23.1 ± 0.6 | 11.4 ± 0.1 | 7.7 ± 0.2 | 43.3 ± 0.7 |
| 500 | 21.4 ± 0.3 | 22.3 ± 0.4 | 11.5 ± 0.3 | 8.1 ± 1.3 | 44.7 ± 2.3 |
| 1000 | 21.7 ± 0.2 | 22.1 ± 0.2 | 10.9 ± 0.4 | 7.4 ± 0.6 | 40.3 ± 0.7 |

RBC=red blood cells. WBC=white blood cells. Each datum is mean±SEM.

For long-term toxicity test of HyPE, a group of 6 mice received a dose of 100 mg HyPEKg body weight, injected IP 3 times a week for 30 weeks (total of 180 mg to a mouse of 20 g). Toxicity was evaluated as for Table 6.1. No mortality, and no significant change in the above criteria was induced by this treatment, compared to normal untreated mice (see Table 6.1), as depicted in Table 6.2.

TABLE 6.2
Results at week 30:

| | Body weight | RBC × | WBC × | Hemato- |
|---|---|---|---|---|
| | (g) | 10⁶ | 10³ | crit % |
| Control (untreated) | 39.5 ± 3.1 | 10.9 ± 0.8 | 9.3 ± 0.6 | 45.0 ± 0.8 |
| rats | | | | |
| HyPE-injected | 39.0 ± 2.7 | 11.7 ± 0.7 | 8.1 ± 15 | 43.4 ± 4.9 |
| rats | | | | |

### Example 7

### Synthesis Procedures

The procedures below are examples for synthesis of specific variants of the lipid-conjugates, and can be modified according to the desirable compositions (e.g., changing the molar ratio between the lipid/phospholipid and the GAG, or the GAG size).

Synthesis of low molecular weight lipid-GAG conjugates are prepared according to US publication 2011-0130555 which is incorporated herein by reference.
I. HyPE=phosphatidyl-ethanolamine (PE)-linked hyaluronic acid.
A. Truncating hyaluronic acid (HA):
   Dissolve 20 g of HA in 12 L water, add 200 mg FeSO₄.7H₂O dissolved in 20 ml water, add 400 ml H₂O₂ (30%), stir for 1.5 h. Filter through 30 kD Filtron, Lyophilize. Yield: 16 g truncated HA.
B. Conjugation with PE (adjusted for 1 g):
Prepare:
   1. 10 g HA dissolved in 500 ml MES buffer, 0.1 M, pH=6.5
   2. 1.0 g PE dissolved in 500 ml t-BuOH with 100 ml H₂O.
Mix the two solutions, add 1 g HOBT and 10 g EDC. Sonicate the mixture in an ultrasonic bath for 3 h. Remove access free PE (and EDC and HOBT) by extraction into organic phase (by addition of chloroform and methanol to obtain a ratio of C/M/H₂0:1/1/1). Separate the aqueous phase by a separation funnel. Repeat this step twice. For final cleaning from reagents, filter through a Filtron membrane (30 kD), and lyophilize.
Yield: about 8 g.
II. CSAPE=PE-linked chondroitin sulfate A (CSA):
Prepare:
   1. 10 g CSA dissolved in 1.2 L MES buffer, 0.1 M, pH=6.5
   2. 1 g PE dissolved in 120 ml chloroform/methanol: 1/1. Add 15 ml of a detergent (DDAB).
Mix 1 with 2, while stirring, add 1 g HOBT and 10 g EDC, continue stirring thoroughly for a day at least. Remove access free PE (and EDC and HOBT) by extraction into organic phase (by addition of chloroform and methanol to obtain a ratio of Chloroform/MeOH/EtOH/H₂O: 1/1/0.75/1). Separate the aqueous phase by a separation funnel. Repeat this step twice. Filter through a Filtron membrane (30 kD), and lyophilize. To remove DDAB traces, dissolve 1 g of dry product in 100 ml water and 100 ml MeOH, and clean by ion exchanger using IR120 resin. Dialyse (to remove MeOH) and lyophilize.
Yield: about 8 g.

Unexpected results showed that the sonication applied in the HyPE synthesis, is an better substitute for the detergent in mixing the aqueous and lipid phases. Using sonication techniques simplifies the synthesis and improves the purification of the product.

It will be appreciated by persons skilled in the art that the present invention is not limited by what has been particularly shown and described herein above and that numerous modifications, all of which fall within the scope of the present invention, exist. Rather, the scope of the invention is defined by the claims which follow:

What is claimed is:

1. A method of treating or preventing bronchitis in a subject, comprising the step of administering to said subject a composition comprising a compound represented by the structure of the general formula (I): 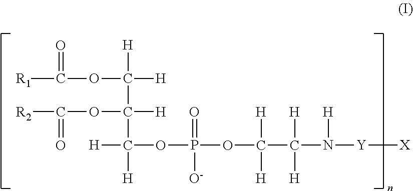
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is glycosaminoglycan, alginate, carboxymethylcellulose, or polygeline; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

2. The method of claim 1, wherein said n is a number from 2 to 1,000.

3. The method of claim 1, wherein said glycosaminoglycan is selected from the group consisting of hyaluronic acid, heparin, heparan sulfate, chondroitin sulfate, keratan, keratan sulfate, dermatan sulfate or a derivative thereof.

4. The method of claim 1, wherein said phosphatidylethanolamine is a myristoyl or palmitoyl phosphatidylethanolamine.

5. The method of claim 1, wherein said phosphatidylethanolamine is a dipalmitoyl phosphatidylethanolamine, or dimyristoyl phosphatidylethanolamine.

6. The method of claim 1, wherein said composition is administered as aerosol.

7. The method of claim 1, wherein said composition is administered by inhalation.

8. The method of claim 1, wherein said composition is administered by intranasal administration.

9. The method of claim 1, wherein said bronchitis is allergic bronchitis, acute bronchitis or chronic bronchitis.

10. A method for ameliorating broncho constriction in a subject, comprising the step of subjecting said subject to a composition comprising a compound represented by the structure of the general formula (I): 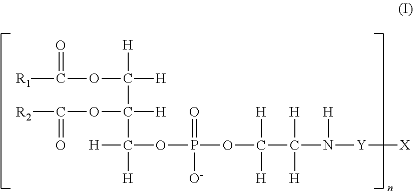
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is glycosaminoglycan, alginate, carboxymethylcellulose, or polygeline; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

11. The method of claim 10, wherein said n is a number from 2 to 1,000.

12. The method of claim 10, wherein said glycosaminoglycan is selected from the group consisting of hyaluronic acid, heparin, heparan sulfate, chondroitin sulfate, keratan, keratan sulfate, dermatan sulfate or a derivative thereof.

13. The method of claim 10, wherein said phosphatidylethanolamine is a myristoyl or palmitoyl phosphatidylethanolamine.

14. The method of claim 10, wherein said phosphatidylethanolamine is a dipalmitoyl phosphatidylethanolamine, or dimyristoyl phosphatidylethanolamine.

15. A method for inhibiting contraction of a muscle tissue, comprising the step of subjecting said muscle tissue to a composition comprising a compound represented by the structure of the general formula (I): 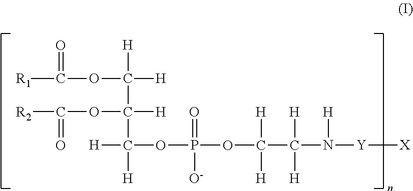
wherein
R₁ is a linear, saturated, mono-unsaturated, or poly-unsaturated, alkyl chain ranging in length from 2 to 30 carbon atoms;
R₂ is a linear, saturated, mono-unsaturated, or poly-unsaturated, alkyl chain ranging in length from 2 to 30 carbon atoms;
Y is either nothing or a spacer group ranging in length from 2 to 30 atoms;
X is glycosaminoglycan, alginate, carboxymethylcellulose, or polygeline; and
n is a number from 1 to 1,000;
wherein if Y is nothing the phosphatidylethanolamine is directly linked to X via an amide bond and if Y is a spacer, said spacer is directly linked to X via an amide or an esteric bond and to said phosphatidylethanolamine via an amide bond.

16. The method of claim 15, wherein said n is a number from 2 to 1,000.

17. The method of claim 15, wherein said glycosaminoglycan is selected from the group consisting of hyaluronic acid, heparin, heparan sulfate, chondroitin sulfate, keratan, keratan sulfate, dermatan sulfate or a derivative thereof.

18. The method of claim 15, wherein said phosphatidylethanolamine is a myristoyl or palmitoyl phosphatidylethanolamine.

19. The method of claim 15, wherein said phosphatidylethanolamine is a dipalmitoyl phosphatidylethanolamine, or dimyristoyl phosphatidylethanolamine.

20. The method of claim 15, wherein said muscle tissue is tracheal ring tissue.


SEQUENCE LISTING (1 sequence)

SEQ ID NO: 1 - DNA, 22 bp, homo sapiens
agttgaggggactttcccaggc